(19) United States
(12) Patent Application Publication
(10) Pub. No.: US 2026/0216294 A1
(43) Pub. Date: Jul. 30, 2026

(54) BETA 2-ADRENERGIC RECEPTOR AGONISTS FOR TREATMENT OR PREVENTION OF MUSCLE WASTING

(71) Applicant: ATROGI AB, Solna (SE)

(72) Inventors: Tore Bengtsson, Vaxholm (SE); Hamza Bokhari, Stockholm (SE); Marcelo Montenegro, Upplands Väsby (SE); Anastasia Kalinovich, Solna (SE)

(21) Appl. No.: 19/148,375

(22) PCT Filed: Jan. 19, 2024
(86) PCT No.: PCT/EP2024/051316

§ 371 Date: Jul. 16, 2025

(30) Foreign Application Priority Data
Jan. 20, 2023 (GB) 2300857.6
Feb. 16, 2023 (GB) 2302194.2
Mar. 6, 2023 (GB) 2303232.9

(51) Int. Cl. A61K 38/26; A61K 31/135; A61K 31/44; A61P 21/06

(52) U.S. Cl. CPC A61K 38/26; A61K 31/135; A61K 31/44; A61P 21/06

(57) ABSTRACT

There is herein provided a β₂-adrenergic receptor agonist, or a pharmaceutically acceptable salt thereof, for use in the treatment or prophylaxis of a disease or disorder characterised by muscle wasting.

### FIELD OF THE INVENTION

The present invention relates to methods for the treatment or prophylaxis of diseases and disorders characterised by muscle wasting, such as muscular dystrophy or muscular atrophy in patients undergoing weight loss treatment. In particular, the invention relates to methods for the treatment or prophylaxis of diseases and disorders characterised by muscle wasting involving treatment with a β₂-adrenergic receptor agonist, and to compositions, combination treatments and kits-of-parts for use in such methods.

### BACKGROUND OF THE INVENTION

The listing or discussion of an apparently prior-published document in this specification should not necessarily be taken as an acknowledgement that the document is part of the state of the art or is common general knowledge.

Muscular atrophy and weakness are pathological manifestations of various neurological, neuromuscular and myogenic disorders (Wei et al., Int J Mol Sci, 21, 9589 (2020)). These can also present themselves as secondary symptoms in conditions such as cancer, cardiopulmonary disorders and are also hallmarks of progressive aging. While gene modification has gained traction as curative therapy for some of these diseases, there is renewed interest in developing novel treatments as adjuncts and for conditions that are not genetic in origin.

At present, pharmacological approaches to the treatment of such diseases and disorders are limited to mitigating secondary and downstream pathological mechanisms. Current research is focused on strategies based on somatic gene editing therapies, which may become effective treatments for muscle wasting disorders resulting from genetic abnormalities. However, considerable progress must be made to improve the delivery and safety of these technologies, and thus far there is no effective treatment on the market.

A related therapeutic area that would benefit from the development of effective treatments for muscle wasting is prevention of muscular atrophy in patients undergoing weight loss treatment.

Obesity is a global health epidemic affecting, with the World Health Organisation reporting that global instances have tripled since 1975. At present, almost 2 billion adults worldwide are affected, leading to serious health concerns.

Numerous treatments have been proposed to address obesity and to generate weight loss in those classified as overweight. These include non-medical interventions, such as adjustments to diet and the adoption of exercise regimes, and medical interventions, such as pharmaceutical and surgical treatments.

Despite ongoing research and a clear public health need, there are still relatively few effective and well-tolerated treatments for obesity, with those treatments approved thus far typically suffering from limitations derived from their mode of action (such as undesirable side effects).

A key feature of weight loss treatments is the initiation of body fat reduction through the restriction of caloric intake and/or stimulation of an increase in metabolism, resulting in the depletion of fat stores. However, an unintended consequence of weight loss treatments is the loss of muscle mass, resulting from catabolism of muscle tissue as an energy source.

Recently, R-agonists have been explored as a potential treatment option for diseases characterised by muscle wasting. Such compounds have been observed to preserve muscle mass and restore muscle function in genetic conditions as Duchenne Muscular Dystrophy (DMD), and Spinal Muscular Atrophy (SMA) and Spinal Bulbar Muscular Atrophy (SBMA) among others. Additionally, these compounds observed to counteract muscle wasting due to disuse as a consequence of injury and aging (Ryall and Lynch, Pharmacol Ther, 120, 219 (2008)). However, there are pitfalls in the use of β-agonists in these diseases primarily due to side effects such as tremors, increased fatiguability and tachycardia, and an eventual loss of efficacy due to β-arrestin recruitment and subsequent receptor desensitization.

The β₂-adreneric receptor is the major isoform of R-adrenergic receptors in skeletal muscle cells. Adrenergic receptors (ARs) are G protein coupled receptors (GPCRs) which signal through classical secondary messengers, such as the canonical cyclic adenosine monophosphate (cAMP)-pathway.

Generally, following β₂-AR stimulation, the receptor couples to the Gas-subunit and activates adenylate cyclase, generating cAMP. Elevated cAMP levels drive multiple parallel signalling pathways that play critical roles in regulating skeletal muscle and CNS, among others.

cAMP activates cAMP-dependent protein kinase A (PKA) which enters the cell nucleus and phosphorylates the ubiquitous transcription factor CREB. Multiple studies have established a key role for CREB in the regulation of muscle mass. CREB regulates the expression of numerous genes involved in skeletal myocyte differentiation, the enhancement of protein synthesis, and inhibition of protein degradation (Bartus R. T. et al., Neurobiology of disease, 2016, 11-24).

Therefore, the hypertrophic and anti-atrophy effects of β₂-AR agonists on skeletal muscle are thought to be mediated by activation of the cAMP/PKA/CREB pathway (Joassard Q. R. et al., Int J Biochem Cell Bio, 2013, 2309; Bartus R. T. et al., Neurobiology of disease, 2016, 11-24).

As cAMP release is linked to side effects such as tremors, increased fatiguability and tachycardia, there are clear disadvantages in relation to the use of β₂-AR agonists for treating muscle wasting (such as preserving muscle mass in a patient undergoing weight loss). A further concern in relation to the use of β₂-AR agonists is an eventual loss of efficacy due to β-arrestin recruitment and subsequent receptor desensitization.

There remains, therefore, a need for new treatments capable of reducing or reversing the symptoms of diseases and disorders characterised by muscle wasting, including treatments capable of treating or preventing loss of muscle mass in patients undergoing weight loss.

### DESCRIPTION OF THE INVENTION

We have now surprisingly found that activation of the β₂-adrenergic receptor (β₂-AR) represents a promising strategy for the treatment and prophylaxis of diseases and disorders characterised by muscle wasting, such as muscular dystrophy or muscular atrophy in patients undergoing weight loss, such as during treatment with a therapeutic agent for the treatment or prophylaxis of obesity, the lowering of body fat composition and/or the reduction of body weight.

Moreover, we have found that such effects can be provided through activation of the β₂-adrenergic receptor without significant production of cAMP. This effect is particularly surprising given that it is believed in the existing literature that β₂-AR agonists provide beneficial hypertrophic and/or anti-atrophy effects by mediating via cAMP (Joassard Q. R. et al., Int J Biochem Cell Bio, 2013, 2309; Bartus R. T. et al., Neurobiology of disease, 2016, 11-24).

Furthermore, such effects have been demonstrated to be achieved without significant recruitment of β-arrestin.

### Medical Treatments

In a first aspect of the invention there is provided a β₂-adrenergic receptor agonist, or a pharmaceutically acceptable salt thereof, for use in the treatment or prophylaxis of a disease or disorder characterised by muscle wasting.

Unless indicated otherwise, all technical and scientific terms used herein have the same meaning as commonly understood by one of ordinary skill in the art to which this invention pertains.

For the avoidance of doubt, β₂-adrenergic receptor agonists, such as those described herein, may be referred to as “compounds of the invention”, or the like.

Preferences and options for a given aspect, embodiment, feature or parameter of the invention should, unless the context indicates otherwise, be regarded as having been disclosed in combination with any and all preferences and options for all other aspects, features and parameters of the invention.

Wherever the word “about” is employed herein (for example, in the context of doses of active ingredients) it will be appreciated that such variables are approximate and as such may vary by ±10%, for example ±5% and preferably ±2% (e.g. ±1%) from the numbers specified herein.

Wherever the word “optionally” is employed in relation to features described herein it will take its normal meaning, namely that the relevant feature may or may not be present.

In an alternative first aspect of the invention there is provided the use of a β₂-adrenergic receptor agonist, or a pharmaceutically acceptable salt thereof, in the manufacture of a medicament for the treatment or prophylaxis of a disease or disorder characterised by muscle wasting.

In an alternative first aspect of the invention there is provided the use of a β₂-adrenergic receptor agonist, or a pharmaceutically acceptable salt thereof, in the treatment or prophylaxis of a disease or disorder characterised by muscle wasting.

In an alternative first aspect of the invention there is provided a method for the treatment or prophylaxis of a disease or disorder characterised by muscle wasting, comprising administration of a therapeutically effective amount of a β₂-adrenergic receptor agonist, or a pharmaceutically acceptable salt thereof, to a patient in need thereof.

For the avoidance of doubt, uses, methods, compositions, combinations and kits-of-parts of other aspects of the invention as described herein (including all embodiments thereof) may have any of the particular features described for the first aspect of the invention, including all combinations thereof.

For the avoidance of doubt, treatments (and associated prophylaxis) as described herein may be referred to herein as the “treatments of the invention”, or the like.

The skilled person will understand that references to the “treatment of” a particular condition (and similarly “treating”) take their normal meanings in the field of medicine.

In particular, the terms may refer to achieving a reduction in the severity of one or more clinical symptom associated with the condition. In particular, the terms may refer to achieving a reduction in the severity of one or more clinical symptom associated with the condition.

As used herein, references to patients will refer to a living subject being treated, including mammalian (e.g. human) patients. In particular embodiments of the relevant aspects of the invention (e.g. the first aspect of the invention), the treatment is in a mammal (e.g. a human).

As used herein, the term therapeutically effective amount will refer to an amount of a compound that confers a therapeutic effect on the treated patient. The effect may be objective (i.e. measurable by some test or marker) or subjective (i.e. the subject gives an indication of and/or feels an effect).

As used herein, the term prophylaxis includes references to the prevention of (and, similarly, preventing) the disease or disorder (and vice-versa). As such, references to prevention may also be references to prophylaxis, and vice versa. In particular, the term may refer to achieving a reduction in the likelihood of the patient (or healthy subject) developing the condition (for example, at least a 10% reduction, such as at least a 20%, 30% or 40% reduction, e.g. at least a 50% reduction).

In particular embodiments, references to use in and methods for the treatment or prophylaxis of diseases and disorders as specified herein will refer in particular to uses in and methods for treatment of (i.e. treating) such diseases and disorders.

For the avoidance of doubt, the term “disease or disorder characterised by muscle wasting” will take its normal meaning in the art, e.g. referring to primary and secondary manifestations of diseases leading to a progressive loss of muscle mass and function.

The skilled person will understand that muscle wasting, as referred to herein, may also be referred to as muscular atrophy, and vice versa. As such, the disease or disorder characterised by muscle wasting may also be referred to as the disease or disorder characterised by muscular atrophy.

The skilled person will understand that references to muscular atrophy will take their usual meaning in the art, such as by referring to the loss of muscle mass (i.e. a decrease in size and/or weight of muscle) and wasting of muscle tissue (e.g. a reduction of muscle strength, i.e. muscle weakening). As such, muscular atrophy may also be referred to herein as loss of muscle mass and/or muscle wasting and/or muscle atrophy.

In particular embodiments, references to muscular atrophy will refer to loss of muscle mass.

In more particular embodiments, references to muscular atrophy will refer to loss of lean mass.

The term “lean muscle mass” will take its usual meaning in the art, such as by referring to the proportion of total body weight of the patient (or subject) that is not fat mass (i.e. Lean Body Mass (LBM) of Patient=Total Weight of Patient−Fat Mass of Patient).

The skilled person will be able to identify levels of muscle mass and lean mass, and therefore monitor changes in the same, using techniques well-known to those skilled in the art.

Without limiting the scope of the invention, the skilled person will understand that symptoms of muscular atrophy may also include muscle numbness and/or tingling in the limbs. In some embodiments, the treatment or prophylaxis of muscular atrophy is in a patient who does not have (for example, has not been diagnosed with, is not experiencing symptoms associated with and/or is not undergoing treatment for) hyperglycaemia or a disorder characterized by hyperglycaemia, such as in a patient who does not have diabetes (e.g. type 2 diabetes), which may be referred to as a non-diabetic (e.g. non-type 2 diabetic) patient.

The skilled person will understand that treatment and prophylaxis of the invention, as described herein, may further comprise (i.e. be combined with) further (i.e. additional/other) treatment(s) for the same condition. Alternatively, the treatment and prophylaxis of the invention, as described herein, may be administered as a monotherapy (i.e. in the absence of other therapeutic agents for the treatment or prophylaxis of the same disease or disorder).

### Compounds of the Invention

As described herein, the present invention requires the use of a β₂-adrenergic receptor agonist, examples of which will be known to those skilled in the art.

The skilled person will understand that compounds referred to herein, such as compounds referred to as agonists, may be provided in the form of a pharmaceutically acceptable salt.

Pharmaceutically acceptable salts include acid addition salts and base addition salts, each of which may be in the form of salts in varying ratios of compound to counter ion (e.g. including hemi salts). Such salts may be formed by conventional means, for example by reaction of a free acid or a free base form of a compound comprised in the formulations of the invention with one or more equivalents of an appropriate acid or base, optionally in a solvent, or in a medium in which the salt is insoluble, followed by removal of said solvent, or said medium, using standard techniques (e.g. by rotary evaporation under reduced pressure, by freeze-drying or by filtration). Salts may also be prepared by exchanging a counter-ion of a compound comprised in the formulations of the invention in the form of a salt with another counter-ion, for example using a suitable ion exchange resin.

Particular acid addition salts that may be mentioned include carboxylate salts (e.g. formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, α-hydroxybutyrate, lactate, tartrate, hemi-tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxybenzoate, salicylate, 1-naphtoate, 2-naphtoate, 1-hydroxy-2-naphtoate, nicotinate, isonicotinate, cinnamate, oxalate, malonate, succinate, suberate, sebacate, fumarate, malate, maleate, hydroxymaleate, hippurate, phthalate or terephthalate salts), halide salts (e.g. chloride, bromide or iodide salts), sulphonate salts (e.g. benzenesulphonate, methyl-, bromo- or chloro-benzenesulphonate, xylenesulphonate, methanesulphonate, ethanesulphonate, propanesulphonate, hydroxyethanesulphonate, 1,2-ethanedisulphonate, 1- or 2-naphthalene-sulphonate or 1,5-naphthalenedisulphonate salts) or sulphate, pyrosulphate, bisulphate, sulphite, bisulphite, phosphate, monohydrogenphosphate, dihydrogenphosphate, metaphosphate, pyrophosphate or nitrate salts, and the like.

Particular base addition salts that may be mentioned include salts formed with alkali metals (such as Na and K salts), alkaline earth metals (such as Mg and Ca salts), organic bases (such as ethanolamine, diethanolamine, triethanolamine, tromethamine and lysine) and inorganic bases (such as ammonia and aluminium hydroxide). More particularly, base addition salts that may be mentioned include Mg, Ca and, most particularly, K and Na salts.

For the avoidance of doubt, compounds as described herein may exist as solids, and thus the scope of the invention includes all amorphous, crystalline and part crystalline forms thereof, and may also exist as oils. Where such compounds exist in crystalline and part crystalline forms, such forms may include solvates, which are included in the scope of the invention. The compounds may also exist in solution.

The skilled person will understand that references to an agonist will refer to compounds suitable for acting as such when administrated to a subject to be treated (i.e. a patient, e.g. a human, in need thereof). Suitable compounds may include compounds which provide the required effect and compounds which are converted to compounds providing the required effect after administration (i.e. in vivo), which compounds may be referred to as pro-drugs. Particular compounds that may be mentioned are compounds which elicit the required effect.

For the avoidance of doubt, the term “agonist” may be understood to indicate an agent (i.e. a compound) that induces activation of the relevant receptor to produce a biological response (e.g. in a subject, such as a human), such as by binding to the relevant receptor. As such, the term may also refer to partial agonists (which will be understood to refer to compounds that activate a given receptor, but have only partial efficacy at the receptor relative to a full agonist).

Agonists (and partial agonists) may display, for example, half maximal effective concentration (EC₅₀) values of less than about 1 mM, such as less than about 100 μM, or less than about 10 μM, such as less than about 1 μM (e.g. less than about 200, about 150, about 120, about 100, about 10 or about 1 nM).

Unless otherwise stated or clear from the context, references herein to agonists will also include pharmaceutically-acceptable (e.g. “protected”) derivatives of compounds which may not possess the relevant activity per se, but may be administered (e.g. parenterally or orally) to a patient and thereafter be metabolised in the body to form compounds possessing the required activity, which compounds may be referred to as prodrugs. Suitable prodrugs of compounds as described herein will be known to those skilled in the art, such as suitable esters (e.g. methyl or ethyl esters, and the like).

For the avoidance of doubt, unless otherwise stated or clear from the context, references to compounds that are agonists, and pharmaceutically acceptable salts thereof, will include compounds that are prodrugs of such agonists, and pharmaceutically acceptable salts thereof.

Suitable β₂-adrenergic receptor agonists (which may also be referred to as β₂-agonists) may include those known to those skilled in the art.

In certain embodiments, suitable β₂-adrenergic receptor agonists will include those that are selective, which term will be known to those skilled in the art (i.e. compounds that are agonists of the relevant receptor(s) but which do not cause significant activation of other R-adrenergic receptors).

Suitable β₂-adrenergic receptor agonists can be identified using techniques known to those skilled in the art, including those as described in the examples provided herein.

Suitable β₂-adrenergic receptor agonists that may be employed in the various aspects of the invention include, but are not limited to, those described in: WO 2004/071388, EP 0 272 976, FR 2647310, DE 2 157 040, DE 2212600, DE 2015573, ZA 6705591, DE 2128258, WO 91/09596, GB 1 199 630, DE 4209989, BE 611502, NL 7804582, EP 0 043 807, WO 2008/022038, DE 2413102, U.S. Pat. No. 2,308,232, BE 823841, BE 660244, WO 2000/075114, WO 2005/102350, WO 2005/110990, JP 56055355, AT 285583, U.S. Pat. Nos. 4,223,137, 3,056,836, FR 1324914, DE 638650, DD 45721, U.S. Pat. No. 3,801,631, DE 2259282, DE 2300614, EP 0 290 122, US 2004/0266867, US 2010/0022658, US 2010/0022659, DE 2157040, GB 2133986, WO 2006/122788, Woo et al., Molecular Pharmacology, (2009), 75(1) 158-165, Baur et al., J. Med. Chem., (2010), 53(9), 3675-3684, Kaiser et al., J. Med. Chem. (1974), 17(1) 49-57, Baker et al., J. Pharmacology and Experimental Therapeutics, (2006), 319(1), 439-446, Engelhardt et al., Arzneimittel-Forschung, (1972), 22(5), 869-76, WO 2019/241744, WO 2019/241736, WO 2020/198466, WO 2021/003161, WO 2021/081292, WO 2021/127210 and WO 2021/247934, the relevant disclosures of each of which (e.g. the examples compounds described therein, and pharmaceutically acceptable salts thereof, and associated methods of preparation) are hereby incorporated by reference in their entirety.

For the avoidance of doubt, references to patent publications will typically refer to the initial publication of the full patent specification with the relevant publication number (which may be indicated by the suffix “A1”).

Further suitable β₂-adrenergic receptor agonists that may be employed in the various aspects of the invention (which compounds may be identified as also being suitable β₂-adrenergic receptor agonists) include those described in the following publications, the contents of which are hereby incorporated herein in their entirety (in particular, the biological examples, the generic compound definitions, including all embodiments thereof and associated definitions, and the example compounds provided therein, including pharmaceutically acceptable salts thereof, and associated methods of preparation):
WO 2017/153737
WO 2019/053429
WO 2019/053426
WO 2019/053425
WO 2019/053427
WO 2020/188299
WO 2020/188301
WO 2022/063895
WO 2022/063889
WO 2023/046885
WO 2023/046882
WO 2023/105035
WO 2023/203223
WO 2020/198466

A particular β₂-adrenergic receptor agonist that may be mentioned is the following compound:

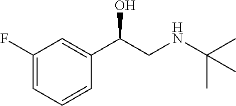

and pharmaceutically acceptable salts thereof.

Thus, a particular β₂-adrenergic receptor agonist that may be mentioned is (R)-2-(tert-butylamino)-1-(3-fluorophenyl)ethan-1-ol, and pharmaceutically acceptable salts thereof.

Particular pharmaceutically acceptable salts of (R)-2-(tert-butylamino)-1-(3-fluorophenyl)ethan-1-ol that may be mentioned include the HCl (hydrochloride) salt.

A further particular β₂-adrenergic receptor agonist that may be mentioned is the following compound:

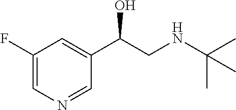

and pharmaceutically acceptable salts thereof.

Thus, a particular β₂-adrenergic receptor agonist that may be mentioned is (R)-2-(tert-butylamino)-1-(5-fluoropyridin-3-yl)ethan-1-ol, and pharmaceutically acceptable salts thereof.

Particular pharmaceutically acceptable salts of the above-mentioned compound (i.e. (R)-2-(tert-butylamino)-1-(5-fluoropyridin-3-yl)ethan-1-ol) that may be mentioned include the hemi-tartrate and dihydrochloride salts (such as the hemi-tartrate salt).

A further particular β₂-adrenergic receptor agonist that may be mentioned is the following compound:

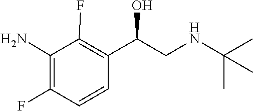 [SMILES: CC(C)(C)NC[C@H](O)c1ccc(F)c(N)c1F]

and pharmaceutically acceptable salts thereof.

Thus, a particular β₂-adrenergic receptor agonist that may be mentioned is (R)-1-(3-amino-2,4-difluorophenyl)-2-(tert-butylamino)-1-ethanol, and pharmaceutically acceptable salts thereof.

Particular pharmaceutically acceptable salts of the above-mentioned compound (i.e. (R)-1-(3-amino-2,4-difluorophenyl)-2-(tert-butylamino)-1-ethanol) that may be mentioned include the hydrochloride, dihydrochloride and maleate salts.

Various β₂-adrenergic receptor agonists are known in the art, such as those selected from the group consisting of formoterol, arformoterol, salmeterol, (R)-salmeterol, vilanterol, zilpaterol, clenbuterol, (R)-clenbuterol, bitolterol, salbutamol, levosalbutamol, terbutaline, metaproterenol, pirbuterol, bambuterol, fenoterol, methoxyfenoterol, isoprenaline, procaterol, ritodrine, indacaterol, olodaterol, colterol, hexaprenaline, carmoterol, isoxsuprine, isoetarine, zinterol, bamethane, (R)-bamethane, clencyclohexerol, tulobuterol, BRL-47672, trantinterol, clenproperol, clenpenterol, brombuterol, ractopamine and abediterol, and pharmaceutically acceptable salts thereof.

Further certain examples of β₂-adrenergic receptor agonists known in the art include those selected from the group consisting of formoterol, arformoterol, salmeterol, (R)-salmeterol, vilanterol, zilpaterol, clenbuterol, (R)-clenbuterol, bitolterol, salbutamol, levosalbutamol, terbutaline, metaproterenol, pirbuterol, bambuterol, fenoterol, methoxyfenoterol, isoprenaline, procaterol, ritodrine, indacaterol, olodaterol, colterol, hexaprenaline, carmoterol, isoxsuprine, isoetarine, zinterol, bamethane, (R)-bamethane, clencyclohexerol, tulobuterol, BRL-47672 and trantinterol, and pharmaceutically acceptable salts thereof.

Particular examples of β₂-adrenergic receptor agonists known in the art include those selected from the group consisting of formoterol, arformoterol, salmeterol, (R)-salmeterol, vilanterol, zilpaterol, clenbuterol, (R)-clenbuterol, indacaterol, olodaterol, carmoterol, bamethane, (R)-bamethane, clencyclohexerol, tulobuterol, trantinerol and abediterol, and pharmaceutically acceptable salts thereof.

Further particular examples of β₂-adrenergic receptor agonists known in the art include those selected from the group consisting of formoterol, arformoterol, salmeterol, (R)-salmeterol, vilanterol, zilpaterol, clenbuterol, (R)-clenbuterol, indacaterol, olodaterol, carmoterol, bamethane, (R)-bamethane, clencyclohexerol, tulobuterol and trantinerol, and pharmaceutically acceptable salts thereof.

More particular examples of β₂-adrenergic receptor agonists known in the art include those selected from the group consisting of formoterol, arformoterol, clenbuterol, (R)-clenbuterol, bamethane, (R)-bamethane, tulobuterol, and abediterol, and pharmaceutically acceptable salts thereof.

Yet more particular examples of β₂-adrenergic receptor agonists known in the art include those selected from the group consisting of formoterol, arformoterol, clenbuterol, (R)-clenbuterol, bamethane, tulobuterol, and (R)-bamethane, and pharmaceutically acceptable salts thereof.

For the avoidance of doubt, the structures of bamethane (CAS: 3703-79-5) and (R)-bamethane (CAS: 912804-58-1) are shown below.

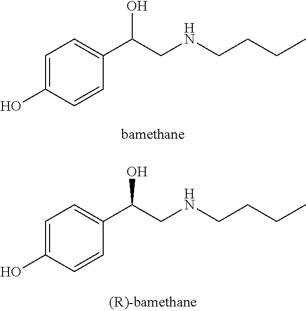

For the avoidance of doubt, in the case of a discrepancy between the name of the compound and the structure drawn in this specification, the structure should prevail.

Further examples of β₂-adrenergic receptor agonists known in the art include those selected from the group consisting of formeterol, arformeterol, clenbuterol, tulobuterol, bambuterol vilanterol, indacaterol, olodaterol, carmoterol and abediterol, and pharmaceutically acceptable salts thereof.

Yet further examples of β₂-adrenergic receptor agonists known in the art include those selected from the group consisting of salbutamol, ritodrine, colterol, hexaprenaline, tulobuterol and isoxsuprine, and pharmaceutically acceptable salts thereof.

Particular examples of β₂-adrenergic receptor agonists known in the art are clenbuterol and (R)-clenbuterol, or a pharmaceutically acceptable salt thereof.

For the avoidance of doubt, the compound clenbuterol may be understood to have the following structure:

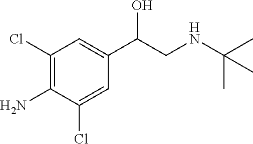 [SMILES: CC(C)(C)NCC(O)c1cc(Cl)c(N)c(Cl)c1]

Particular examples of β₂-adrenergic receptor agonists known in the art are tulobuterol and (R)-tulobuterol, or a pharmaceutically acceptable salt thereof.

For the avoidance of doubt, the compound tulobuterol may be understood to have the following structure:

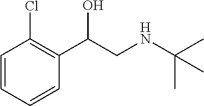

For the avoidance of doubt, the international nonpropriety name (INN) or developmental drug code (e.g. BRL-47672) for a compound generally indicates the stereochemical configuration of the compound, or a particular mixture of stereoisomers (e.g. a racemate). Within the scope of the present invention, where relevant and unless context indicates otherwise (for example where both the racemate and a single stereoisomer are explicitly named), such names may also be considered to encompass separate stereoisomers that display the relevant biological activity, and which have not presently been assigned an alternative INN or developmental drug code.

In particular embodiments, the INN or developmental drug code should be understood to represent the compound to which the relevant name or code has been assigned only.

Where no INN or developmental drug code is available for a compound, the compound may be identified by its Chemical Abstracts Service Registry Number (CAS number).

As referred to herein, the indication “CAS: XXXXXX-XX-X” (wherein the number of figures in the first group may vary) is used to identify such compounds. Where relevant and unless context indicates otherwise, the CAS number for a compound may also be considered to encompass other stereoisomers, or mixtures thereof, that display the relevant biological activity, and which have not presently been assigned alternative CAS numbers (as described above for INNs and developmental drug codes).

In particular embodiments, the CAS number should be understood to represent the compound to which the relevant name or code has been assigned only.

In particular embodiments, the β₂-adrenergic receptor agonist is selected from the group consisting of (R)-bamethane, bamethane, clencyclohexerol, radopamine, tulobuterol, and (R)-tulobuterol.

In particular embodiments, references to a specific stereoisomer of a compound may refer to the specific stereoisomer being present (e.g. in a composition or formulation comprising the same) in the substantial absence of the corresponding opposite stereoisomer.

As used herein, references to the substantial absence of the corresponding opposite stereoisomer may refer to the desired stereoisomer being present at a purity of at least 80% (e.g. at least 90%, such as at least 95%) relative to the opposite stereoisomer.

Alternatively, in such instances, compounds may be indicated to be present in the substantial absence of the compound in the other configuration, which may indicate that the compound in the relevant configuration is present in an enantiomeric excess (e.e.) of at least 80% (such as at least 90%, at least 95%, at least 98% or, particularly, at least 99%, for example at least 99.9%).

The present invention also embraces pharmaceutical formulations comprising isotopically-labelled compounds, which are identical to the compounds recited herein but for the fact that one or more atoms are replaced by an atom having an atomic mass or mass number different from the atomic mass or mass number usually found in nature (or the most abundant one found in nature). All isotopes of any particular atom or element as specified herein are contemplated within the scope of the compounds of the invention. Hence, the invention also encompasses pharmaceutical formulations comprising deuterated compounds, i.e. in which one or more hydrogen atoms are replaced by the hydrogen isotope deuterium.

As described herein, it will also be understood that certain compounds acting as β₂-adrenergic receptor agonists are able to activate the β₂-adrenergic receptor without inducing significant cAMP production.

Thus, particular β₂-adrenergic receptor agonists that may be mentioned include those able to activate the β₂-adrenergic receptor without (or with only a minimal effect in) inducing cAMP production.

Further, in particular embodiments, the methods and uses as described herein may be performed without inducing (or without inducing significant levels of) cAMP production.

In particular, the β₂-adrenergic receptor agonist as described herein may be further described as being a β₂-adrenergic receptor agonist that does not induce significant cAMP (i.e. levels and/or production thereof).

The skilled person will be able to determine the level of cAMP production provided by compounds, such as those referred to herein, using techniques known to those skilled in the art, such as those described in the examples as provided herein.

For example, the level of cAMP production induced by a given compound can be determined by reference to the amount induced by a defined concentration of that compound relative to that induced by the same concentration of a reference compound, such as isoprenaline, using techniques known to those skilled in the art (e.g. following the protocol as described in the biological examples provided herein, i.e. in cells, such as differentiated L6-myotubes, having been stimulated with isoprenaline or the compound with a final concentration of 1×10⁻⁵ M, for 15 min in stimulation buffer, such as HBSS supplemented with 1% BSA, 5 mM HEPES and 1 mM IBMX, at pH 7.4).

In such embodiments, compounds that do not induce significant cAMP may be defined as being compounds that induce less than 50% (or, in some embodiments, less than 25%) of the cAMP induced by isoprenaline (e.g. in accordance with the protocol described above).

Particular compounds acting as β₂-adrenergic receptor agonists which are able to activate the β₂-adrenergic receptor without (or with only a minimal effect in) inducing cAMP production include those described in the following publications, the contents of which are incorporated herein in their entirety (in particular, the biological examples, the generic compound definitions, including all embodiments thereof and associated definitions, and the example compounds provided therein, including pharmaceutically acceptable salts thereof, and associated methods of preparation):
WO 2017/153737
WO 2019/053429
WO 2019/053426
WO 2019/053425
WO 2019/053427
WO 2020/188299
WO 2020/188301
WO 2022/063895
WO 2022/063889
WO 2023/046885
WO 2023/046882
WO 2023/105035
WO 2023/203223

More particular compounds acting as β₂-adrenergic receptor agonists which are able to activate the β₂-adrenergic receptor without (or with only a minimal effect in) inducing cAMP production include those described in the following publications, the contents of which are incorporated herein in their entirety (in particular, the biological examples, the generic compound definitions, including all embodiments thereof and associated definitions, and the example compounds provided therein, including pharmaceutically acceptable salts thereof, and associated methods of preparation):
WO 2020/198466
U.S. Ser. No. 10/947,196
U.S. Ser. No. 11/040,944
WO 2021/247934

Compounds described in publications referenced and incorporated herein were screened in accordance with the procedures outlined in Biological example 2 and Biological example 3. Test results are given in Table 1. If a compound at 10 μM shows activity of more than 75% of that of isoproterenol at 10 μM, the activity is denoted with +++; if it is between 75 and 50% it is denoted with ++; if it is between 50 and 25% it is denoted with +; if it less than 25% it is denoted with −. Compounds are drawn as salt-free molecules in Table 1, but the tested compounds might have been containing additional salt or solvent components that do not contribute to the biological activity.

In the event that there is a discrepancy between nomenclature and the structure of compounds as depicted graphically, it is the latter that presides (unless contradicted by any experimental details that may be given and/or unless it is clear from the context). Synthetic procedures and further biological data will be found in the relevant publications.

Therefore, particular β₂-adrenergic receptor agonists that may be mentioned are provided in Table 1 below.

TABLE 1
Particular compounds of the invention

| | | | Biological | Biological |
|---|---|---|---|---|
| | | | example 2 | example 3 |
| | Structure | Name | (GU) | (CAMP) |
| 1 | 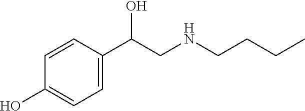 | 2-(butylamino)-1-(p- hydroxyphenyl)-1- ethanol | +++ | |
| 2 | 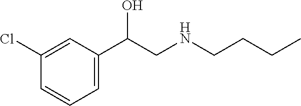 | 2-(butylamino)-1-(m- chlorophenyl)-1-ethanol | + | − |
| 3 | 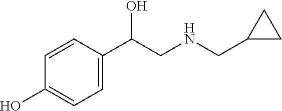 [SMILES: Oc1ccc(C(O)CNCC2CC2)cc1] | p-{2- [(cyclopropylmethyl) amino]-1- hydroxyethyl}phenol | ++ | − |
| 4 | 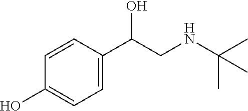 | 2-(tert-butylamino)-1- (p-hydroxyphenyl)-1- ethanol | +++ | + |
| 5 | 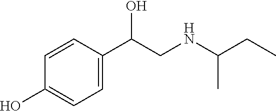 | 2-(sec-butylamino)-1- (p-hydroxyphenyl)-1- ethanol | ++ | − |
| 6 | 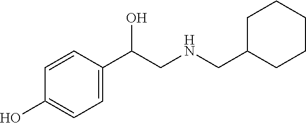 [SMILES: Oc1ccc(C(O)CNCC2CCCCC2)cc1] | 2- [(cyclohexylmethyl) amino]-1-(p- hydroxyphenyl)-1- ethanol | +++ | − |
| 7 | 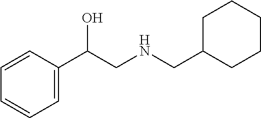 [SMILES: OC(CNCC1CCCCC1)c1ccccc1] | 2- [(cyclohexylmethyl) amino]-1-phenyl-1- ethanol | + | − |
| 8 | 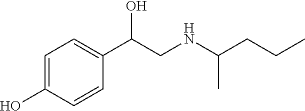 [SMILES: CCCC(C)NCC(O)c1ccc(O)cc1] | 1-(p-hydroxyphenyl)-2- (1-methylbutylamino)- 1-ethanol | ++ | + |
| 9 | 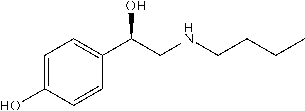 | (R)-2-(butylamino)-1- (p-hydroxyphenyl)-1- ethanol | +++ | − |
| 10 | 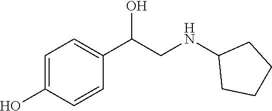 [SMILES: Oc1ccc(C(O)CNC2CCCC2)cc1] | p-[2- (cyclopentylamino)-1- hydroxyethyl]phenol | ++ | − |
| 11 | 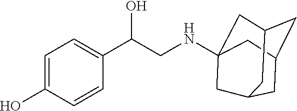 [SMILES: Oc1ccc(C(O)CNC23CC4CC(CC(C4)C2)C3)cc1] | p-[2-(1- adamantanylamino)-1- hydroxyethyl]phenol | + | − |
| 12 | 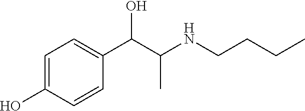 [SMILES: CCCCNC(C)C(O)c1ccc(O)cc1] | 2-(butylamino)-1-(p- hydroxyphenyl)-1- propanol | ++ | − |
| 13 | 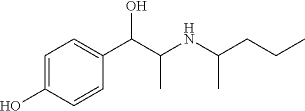 [SMILES: CCCC(C)NC(C)C(O)c1ccc(O)cc1] | 1-(p-hydroxyphenyl)-2- (1-methylbutylamino)- 1-propanol | ++ | − |
| 14 | 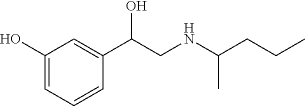 [SMILES: CCCC(C)NCC(O)c1cccc(O)c1] | 1-(m-hydroxyphenyl)-2- (1-methylbutylamino)- 1-ethanol | +++ | − |
| 15 | 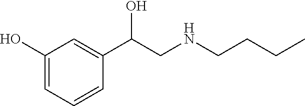 | 2-(butylamino)-1-(m- hydroxyphenyl)-1- ethanol | ++ | − |
| 16 | 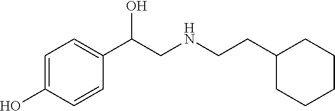 [SMILES: Oc1ccc(C(O)CNCCC2CCCCC2)cc1] | 2-(2- cyclohexylethylamino)- 1-(p-hydroxyphenyl)-1- ethanol | + | − |
| 17 | 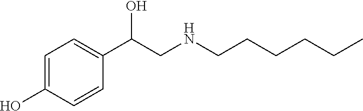 [SMILES: CCCCCCNCC(O)c1ccc(O)cc1] | 2-(hexylamino)-1-(p- hydroxyphenyl)-1- ethanol | + | − |
| 18 | 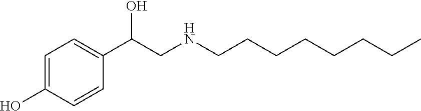 [SMILES: CCCCCCCCNCC(O)c1ccc(O)cc1] | 1-(p-hydroxyphenyl)-2- (octylamino)-1-ethanol | + | − |
| 19 | 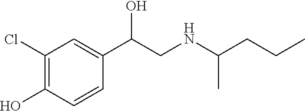 [SMILES: CCCC(C)NCC(O)c1ccc(O)c(Cl)c1] | 1-(3-chloro-4- hydroxyphenyl)-2-(1- methylbutylamino)-1- ethanol | + | − |
| 20 | 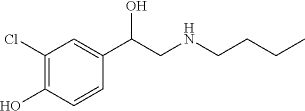 [SMILES: CCCCNCC(O)c1ccc(O)c(Cl)c1] | 2-(butylamino)-1-(3- chloro-4- hydroxyphenyl)-1- ethanol | + | − |
| 21 | 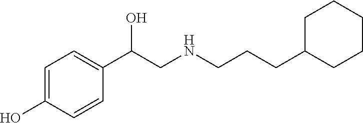 [SMILES: Oc1ccc(C(O)CNCCCC2CCCCC2)cc1] | 2-(3- cyclohexylpropylamino)- 1-(p-hydroxyphenyl)-1- ethanol | +++ | − |
| 22 | 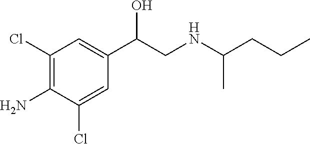 [SMILES: CCCC(C)NCC(O)c1cc(Cl)c(N)c(Cl)c1] | 1-(4-amino-3,5- dichlorophenyl)-2-(1- methylbutylamino)-1- ethanol | +++ | ++ |
| 23 | 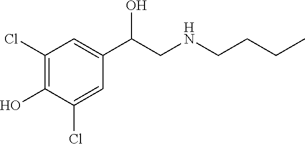 [SMILES: CCCCNCC(O)c1cc(Cl)c(O)c(Cl)c1] | 2-(butylamino)-1-(3,5- dichloro-4- hydroxyphenyl)-1- ethanol | − | − |
| 24 | 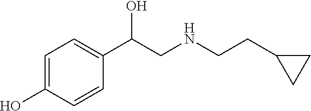 [SMILES: Oc1ccc(C(O)CNCCC2CC2)cc1] | p-[2-(2- cyclopropylethylamino)- 1-hydroxyethyl]phenol | ++ | − |
| 25 | 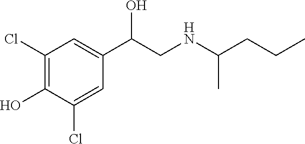 [SMILES: CCCC(C)NCC(O)c1cc(Cl)c(O)c(Cl)c1] | 1-(3,5-dichloro-4- hydroxyphenyl)-2-(1- methylbutylamino)-1- ethanol | − | − |
| 26 | 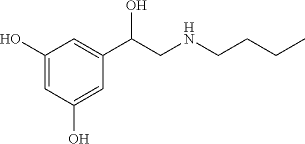 [SMILES: CCCCNCC(O)c1cc(O)cc(O)c1] | 5-[2-(butylamino)-1- hydroxyethyl]resorcinol | ++ | − |
| 27 | 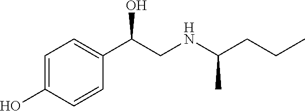 [SMILES: CCC[C@@H](C)NC[C@H](O)c1ccc(O)cc1] | (R)-2-[(R)-1- methylbutylamino]-1- (p-hydroxyphenyl)-1- ethanol | +++ | + |
| 28 | 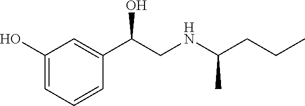 [SMILES: CCC[C@@H](C)NC[C@H](O)c1cccc(O)c1] | (R)-2-[(R)-1- methylbutylamino]-1- (m-hydroxyphenyl)-1- ethanol | +++ | ++ |
| 29 | 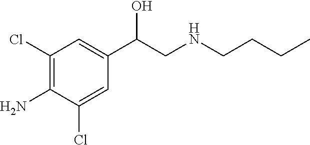 [SMILES: CCCCNCC(O)c1cc(Cl)c(N)c(Cl)c1] | 1-(4-amino-3,5- dichlorophenyl)-2- (butylamino)-1-ethanol | +++ | + |
| 30 | 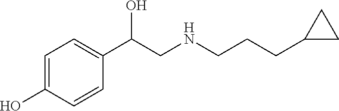 [SMILES: Oc1ccc(C(O)CNCCCC2CC2)cc1] | 2-(3- cyclopropylpropylamino) -1-(p-hydroxyphenyl)- 1-ethanol | +++ | − |
| 31 | 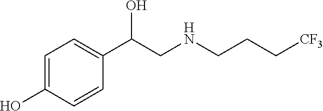 [SMILES: Oc1ccc(C(O)CNCCCC(F)(F)F)cc1] | 1-(p-hydroxyphenyl)-2- (4,4,4- trifluorobutylamino)-1- ethanol | ++ | n/a |
| 32 | 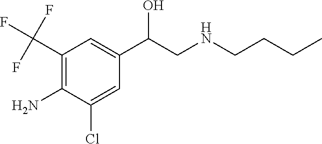 [SMILES: CCCCNCC(O)c1cc(Cl)c(N)c(C(F)(F)F)c1] | 1-[4-amino-3-chloro-5- (trifluoromethyl)phenyl] -2-(butylamino)-1- ethanol | +++ | + |
| 33 | 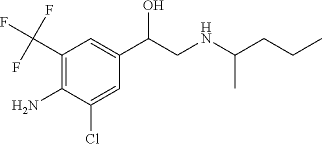 [SMILES: CCCC(C)NCC(O)c1cc(Cl)c(N)c(C(F)(F)F)c1] | 1-[4-amino-3-chloro-5- (trifluoromethyl)phenyl] -2-(1- methylbutylamino)-1- ethanol | ++ | +++ |
| 34 | 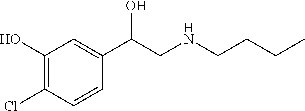 [SMILES: CCCCNCC(O)c1ccc(Cl)c(O)c1] | 2-(butylamino)-1-(4- chloro-3- hydroxyphenyl)-1- ethanol | +++ | − |
| 35 | 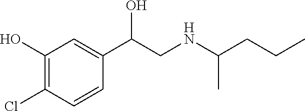 [SMILES: CCCC(C)NCC(O)c1ccc(Cl)c(O)c1] | 1-(4-chloro-3- hydroxyphenyl)-2-(1- methylbutylamino)-1- ethanol | +++ | − |
| 36 | 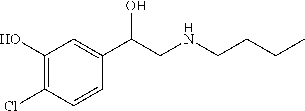 [SMILES: CCCCNCC(O)c1ccc(Cl)c(O)c1] | 1-(3-amino-4- chlorophenyl)-2- (butylamino)-1-ethanol | + | − |
| 37 | 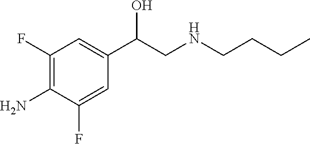 [SMILES: CCCCNCC(O)c1cc(F)c(N)c(F)c1] | 1-(4-amino-3,5- difluorophenyl)-2- (butylamino)-1-ethanol | +++ | +++ |
| 38 | 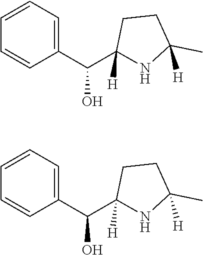 | Racemic mixture of (R)- [(2S,5S)-5-methyl-2- pyrrolidinyl]phenylmeth anol and (S)-[(2R,5R)- 5-methyl-2- pyrrolidinyl]] phenylmethanol | + | − |
| 39 | 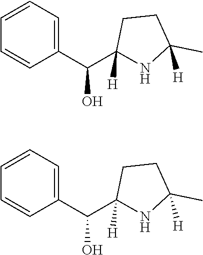 | Racemic mixture of (R)- [(2R,5R)-5-methyl-2- pyrrolidinyl] phenylmethanol and (S)-[(2S,5S)- 5-methyl-2- pyrrolidinyl] phenylmethanol | ++ | − |
| 40 | 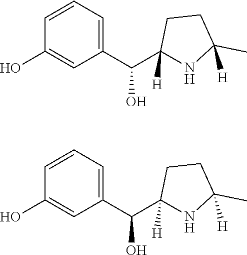 | Racemic mixture of m- {(R)-[(2S,5S)-5- methyl-2- pyrrolidinyl] hydroxymethyl} phenol and m-{(S)- [(2R,5R)-5-methyl-2- pyrrolidinyl] hydroxymethyl} phenol | ++ | − |
| 41 | 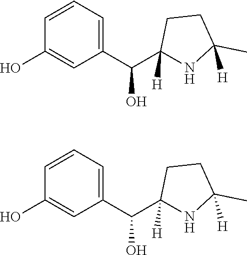 | Racemic mixture of m- {(R)-[(2R,5R)-5- methyl-2- pyrrolidinyl] hydroxymethyl}phenol and m-{(S)- [(2S,5S)-5-methyl-2- pyrrolidinyl] hydroxymethyl}phenol | +++ | − |
| 42 | 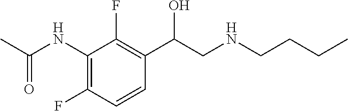 [SMILES: CCCCNCC(O)c1ccc(F)c(NC(C)=O)c1F] | N-{3-[2-(butylamino)- 1-hydroxyethyl]-2,6- difluorophenyl}acetamide | + | − |
| 43 | 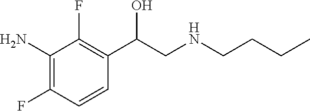 [SMILES: CCCCNCC(O)c1ccc(F)c(N)c1F] | 1-(3-amino-2,4- difluorophenyl)-2- (butylamino)-1-ethanol | +++ | − |
| 44 | 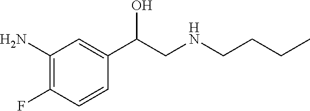 [SMILES: CCCCNCC(O)c1ccc(F)c(N)c1] | 1-(3-amino-4- fluorophenyl)-2- (butylamino)-1-ethanol | ++ | − |
| 45 | 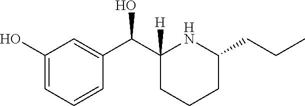 [SMILES: CCC[C@@H]1CCC[C@H]([C@H](O)c2cccc(O)c2)N1] | (R)-[(2R,6R)-6-propyl- 2-piperidyl]](m- hydroxyphenyl)methanol | +++ | − |
| 46 | 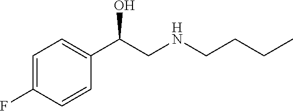 | (R)-2-(butylamino)-1- (p-fluorophenyl)-1- ethanol | ++ | − |
| 47 | 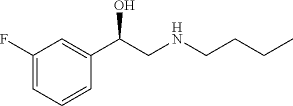 | (R)-2-(butylamino)-1- (m-fluorophenyl)-1- ethanol | ++ | − |
| 48 | 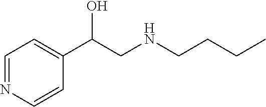 | 2-(butylamino)-1-(4- pyridyl)-1-ethanol | + | − |
| 49 | 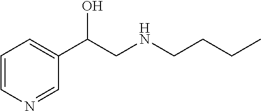 | 2-(butylamino)-1-(3- pyridyl)-1-ethanol | ++ | − |
| 50 | 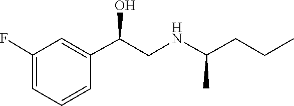 [SMILES: CCC[C@@H](C)NC[C@H](O)c1cccc(F)c1] | (R)-2-[(R)-1- methylbutylamino]-1- (m-fluorophenyl)-1- ethanol | +++ | − |
| 51 | 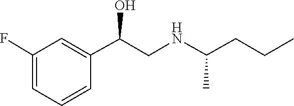 [SMILES: CCC[C@H](C)NC[C@H](O)c1cccc(F)c1] | (R)-2-[(S)-1- methylbutylamino]]-1- (m-fluorophenyl)-1- ethanol | +++ | − |
| 52 | 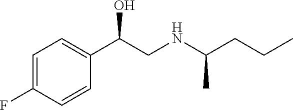 [SMILES: CCC[C@@H](C)NC[C@H](O)c1ccc(F)cc1] | (R)-2-[(R)-1- methylbutylamino]-1- (p-fluorophenyl)-1- ethanol | ++ | − |
| 53 | 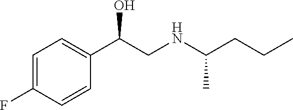 [SMILES: CCC[C@H](C)NC[C@H](O)c1ccc(F)cc1] | (R)-2-[(S)-1- methylbutylamino]-1- (p-fluorophenyl)-1- ethanol | ++ | − |
| 54 | 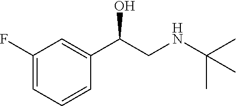 | (R)-2-(tert-butylamino)- 1-(m-fluorophenyl)-1- ethanol | +++ | − |
| 55 | 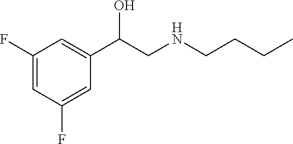 [SMILES: CCCCNCC(O)c1cc(F)cc(F)c1] | 2-(butylamino)-1-(3,5- difluorophenyl)-1- ethanol | ++ | − |
| 56 | 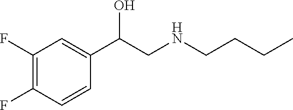 [SMILES: CCCCNCC(O)c1ccc(F)c(F)c1] | 2-(butylamino)-1-(3,4- difluorophenyl)-1- ethanol | ++ | − |
| 57 | 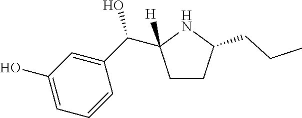 [SMILES: CCC[C@@H]1CC[C@H]([C@@H](O)c2cccc(O)c2)N1] | m-{(R)-[(2R,5R)-5- propyl-2- pyrrolidinyl] hydroxymethyl}phenol | +++ | ++ |
| 58 | 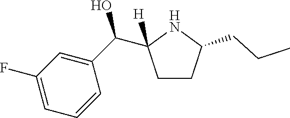 [SMILES: CCC[C@@H]1CC[C@H]([C@H](O)c2cccc(F)c2)N1] | (R)-[(2R,5R)-5-propyl- 2-pyrrolidinyl]](m- fluorophenyl)methanol | +++ | + |
| 59 | 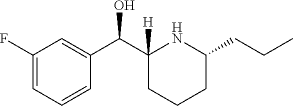 [SMILES: CCC[C@@H]1CCC[C@H]([C@H](O)c2cccc(F)c2)N1] | (R)-[(2R,6R)-6-propyl- 2-piperidyl](m- fluorophenyl)methanol | + | − |
| 60 | 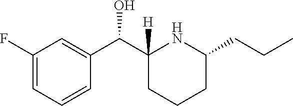 [SMILES: CCC[C@@H]1CCC[C@H]([C@@H](O)c2cccc(F)c2)N1] | (S)-[(2R,6R)-6-propyl- 2-piperidyl]](m- fluorophenyl)methanol | + | − |
| 61 | 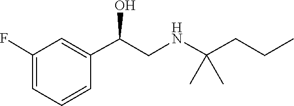 [SMILES: CCCC(C)(C)NC[C@H](O)c1cccc(F)c1] | (R)-2-(1,1- dimethylbutylamino)-1- (m-fluorophenyl)-1- ethanol | +++ | − |
| 62 | 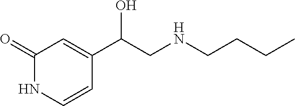 | 4-[2-(butylamino)-1- hydroxyethyl]-2(1H)- pyridinone | + | − |
| 63 | 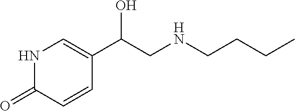 | 5-[2-(butylamino)-1- hydroxyethyl]-2(1H)- pyridinone | + | − |
| 64 | 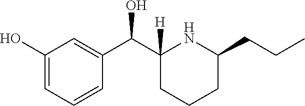 [SMILES: CCC[C@H]1CCC[C@H]([C@H](O)c2cccc(O)c2)N1] | (R)-[(2R,6S)-6-propyl- 2-piperidyl](m- hydroxyphenyl)methanol | +++ | − |
| 65 | 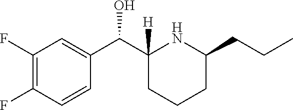 [SMILES: CCC[C@H]1CCC[C@H]([C@@H](O)c2ccc(F)c(F)c2)N1] | (S)-[(2R,6S)-6-propyl- 2-piperidyl]](3,4- difluorophenyl)methanol | + | − |
| 66 | 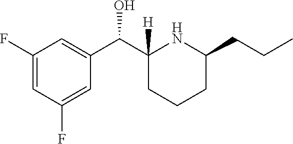 [SMILES: CCC[C@H]1CCC[C@H]([C@@H](O)c2cc(F)cc(F)c2)N1] | (S)-[(2R,6S)-6-propyl- 2-piperidyl]](3,5- difluorophenyl)methanol | + | − |
| 67 | 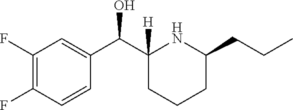 [SMILES: CCC[C@H]1CCC[C@H]([C@H](O)c2ccc(F)c(F)c2)N1] | (R)-[(2R,6S)-6-propyl- 2-piperidyl](3,4- difluorophenyl)methanol | + | − |
| 68 | 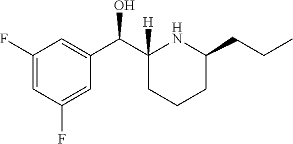 [SMILES: CCC[C@H]1CCC[C@H]([C@H](O)c2cc(F)cc(F)c2)N1] | (R)-[(2R,6S)-6-propyl- 2-piperidyl]](3,5- difluorophenyl)methanol | ++ | − |
| 69 | 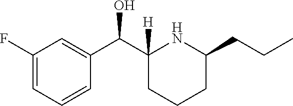 [SMILES: CCC[C@H]1CCC[C@H]([C@H](O)c2cccc(F)c2)N1] | (R)-[(2R,6S)-6-propyl- 2-piperidyl]](m- fluorophenyl)methanol | ++ | − |
| 70 | 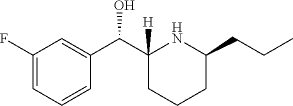 [SMILES: CCC[C@H]1CCC[C@H]([C@@H](O)c2cccc(F)c2)N1] | (S)-[(2R,6S)-6-propyl- 2-piperidyl]](m- fluorophenyl)methanol | ++ | − |
| 71 | 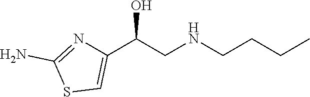 | (S)-1-(2-amino-1,3- thiazol-4-yl)-2- (butylamino)-1-ethanol | + | − |
| 72 | 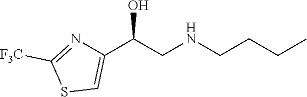 [SMILES: CCCCNC[C@H](O)c1csc(C(F)(F)F)n1] | (S)-2-(butylamino)-1- [2-(trifluoromethyl)- 1,3-thiazol-4-yl]-1- ethanol | + | − |
| 73 | 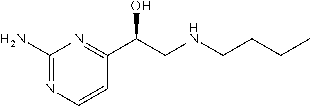 | (S)-1-(2-amino-4- pyrimidinyl)-2- (butylamino)-1-ethanol | + | − |
| 74 | 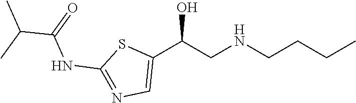 [SMILES: CCCCNC[C@H](O)c1cnc(NC(=O)C(C)C)s1] | N-{5-[(S)-2- (butylamino)-1- hydroxyethyl]-1,3- thiazol-2-yl}2- methylpropionamide | + | − |
| 75 | 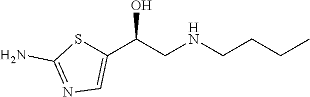 | (S)-1-(2-amino-1,3- thiazol-5-yl)-2- (butylamino)-1-ethanol | + | − |
| 76 | 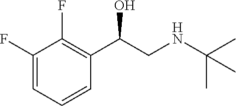 [SMILES: CC(C)(C)NC[C@H](O)c1cccc(F)c1F] | (R)-2-(tert-butylamino)- 1-(2,3-difluorophenyl)- 1-ethanol | +++ | + |
| 77 | 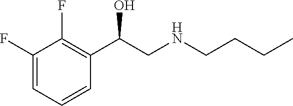 [SMILES: CCCCNC[C@H](O)c1cccc(F)c1F] | (R)-2-(butylamino)-1- (2,3-difluorophenyl)-1- ethanol | +++ | − |
| 78 | 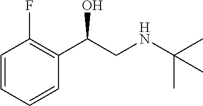 | (R)-2-(tert-butylamino)- 1-(o-fluorophenyl)-1- ethanol | +++ | + |
| 79 | 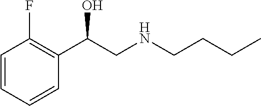 | (R)-2-(butylamino)-1- (o-fluorophenyl)-1- ethanol | +++ | − |
| 80 | 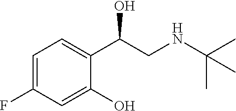 [SMILES: CC(C)(C)NC[C@H](O)c1ccc(F)cc1O] | (R)-2-(tert-butylamino)- 1-(4-fluoro-2- hydroxyphenyl)-1- ethanol | ++ | − |
| 81 | 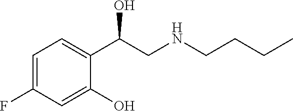 [SMILES: CCCCNC[C@H](O)c1ccc(F)cc1O] | (R)-2-(butylamino)-1- (4-fluoro-2- hydroxyphenyl)-1- ethanol | ++ | − |
| 82 | 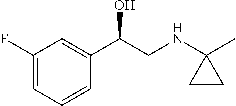 | (R)-1-(m-fluorophenyl)- 2-(1- methylcyclopropylamino)- 1-ethanol | +++ | − |
| 83 | 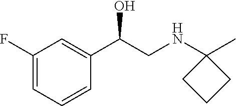 [SMILES: CC1(NC[C@H](O)c2cccc(F)c2)CCC1] | (R)-1-(m-fluorophenyl)- 2-(1- methylcyclobutylamino)- 1-ethanol | +++ | + |
| 84 | 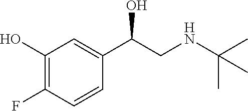 [SMILES: CC(C)(C)NC[C@H](O)c1ccc(F)c(O)c1] | (R)-2-(tert-butylamino)- 1-(4-fluoro-3- hydroxyphenyl)-1- ethanol | +++ | ++ |
| 85 | 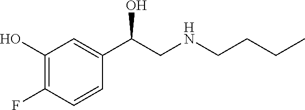 [SMILES: CCCCNC[C@H](O)c1ccc(F)c(O)c1] | (R)-2-(butylamino)-1- (4-fluoro-3- hydroxyphenyl)-1- ethanol | +++ | − |
| 86 | 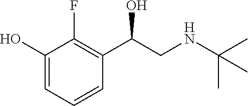 [SMILES: CC(C)(C)NC[C@H](O)c1cccc(O)c1F] | (R)-2-(tert-butylamino)- 1-(2-fluoro-3- hydroxyphenyl)-1- ethanol | ++ | ++ |
| 87 | 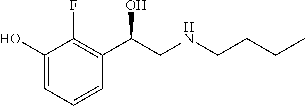 [SMILES: CCCCNC[C@H](O)c1cccc(O)c1F] | (R)-2-(butylamino)-1- (2-fluoro-3- hydroxyphenyl)-1- ethanol | +++ | + |
| 88 | 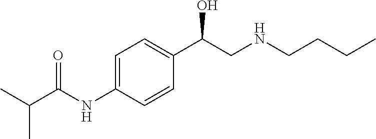 [SMILES: CCCCNC[C@H](O)c1ccc(NC(=O)C(C)C)cc1] | N-{5-[(R)-2- (butylamino)-1- hydroxyethyl]-2- pyrimidinyl}2- methylpropionamide | ++ | − |
| 89 | 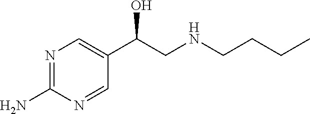 | (R)-1-(2-amino-5- pyrimidinyl)-2- (butylamino)-1-ethanol | ++ | − |
| 90 | 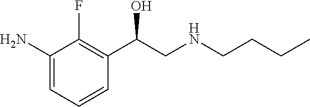 [SMILES: CCCCNC[C@H](O)c1cccc(N)c1F] | (R)-1-(3-amino-2- fluorophenyl)-2- (butylamino)-1-ethanol | ++ | − |
| 91 | 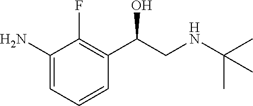 [SMILES: CC(C)(C)NC[C@H](O)c1cccc(N)c1F] | (R)-1-(3-amino-2- fluorophenyl)-2-(tert- butylamino)-1-ethanol | +++ | + |
| 92 | 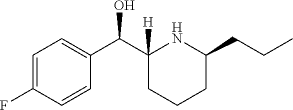 [SMILES: CCC[C@H]1CCC[C@H]([C@H](O)c2ccc(F)cc2)N1] | (R)-[(2R,6S)-6-propyl- 2-piperidyl]](p- fluorophenyl)methanol | + | − |
| 93 | 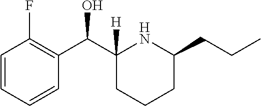 [SMILES: CCC[C@H]1CCC[C@H]([C@H](O)c2ccccc2F)N1] | (R)-[(2R,6S)-6-propyl- 2-piperidyl](o- fluorophenyl)methanol | + | − |
| 94 | 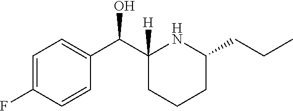 [SMILES: CCC[C@@H]1CCC[C@H]([C@H](O)c2ccc(F)cc2)N1] | (R)-[(2R,6R)-6-propyl- 2-piperidyl]](p- fluorophenyl)methanol | ++ | − |
| 95 | 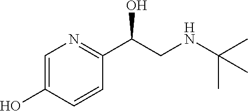 | 6-[(S)-2-(tert- butylamino)-1- hydroxyethyl]-3- pyridinol | ++ | − |
| 96 | 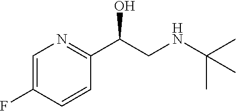 | (S)-2-(tert-butylamino)- 1-(5-fluoro-2-pyridyl)- 1-ethanol | ++ | − |
| 97 | 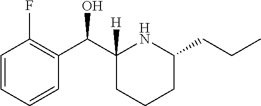 [SMILES: CCC[C@@H]1CCC[C@H]([C@H](O)c2ccccc2F)N1] | (R)-[(2R,6R)-6-propyl- 2-piperidyl](o- fluorophenyl)methanol | ++ | − |
| 98 | 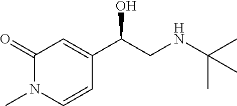 [SMILES: Cn1ccc([C@@H](O)CNC(C)(C)C)cc1=O] | 4-[(R)-2-(tert- butylamino)-1- hydroxyethyl]-1- methyl-2(1H)- pyridinone | +++ | − |
| 99 | 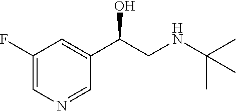 | (R)-2-(tert-butylamino)- 1-(5-fluoro-3-pyridyl)- 1-ethanol | +++ | + |
| 100 | 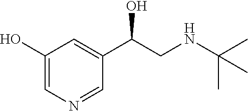 | 5-[(R)-2-(tert- butylamino)-1- hydroxyethyl]-3- pyridinol | +++ | − |
| 101 | 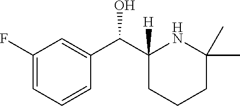 [SMILES: CC1(C)CCC[C@H]([C@@H](O)c2cccc(F)c2)N1] | (S)-[(R)-6,6-dimethyl- 2-piperidyl]](m- fluorophenyl)methanol | + | − |
| 102 | 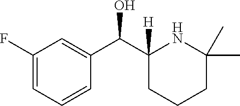 [SMILES: CC1(C)CCC[C@H]([C@H](O)c2cccc(F)c2)N1] | (R)-[(R)-6,6-dimethyl- 2-piperidyl]](m- fluorophenyl)methanol | ++ | − |
| 103 | 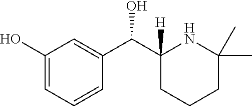 [SMILES: CC1(C)CCC[C@H]([C@@H](O)c2cccc(O)c2)N1] | (S)-[(R)-6,6-dimethyl- 2-piperidyl]](m- hydroxyphenyl)methanol | + | − |
| 104 | 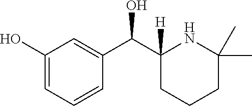 [SMILES: CC1(C)CCC[C@H]([C@H](O)c2cccc(O)c2)N1] | (R)-[(R)-6,6-dimethyl- 2-piperidyl](m- hydroxyphenyl)methanol | + | − |
| 105 | 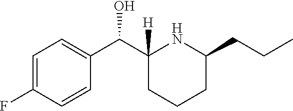 [SMILES: CCC[C@H]1CCC[C@H]([C@@H](O)c2ccc(F)cc2)N1] | (S)-[(2R,6S)-6-propyl- 2-piperidyl]](p- fluorophenyl)methanol | + | − |
| 106 | 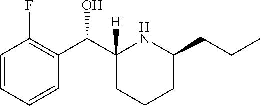 [SMILES: CCC[C@H]1CCC[C@H]([C@@H](O)c2ccccc2F)N1] | (S)-[(2R,6S)-6-propyl- 2-piperidyl](o- fluorophenyl)methanol | + | − |
| 107 | 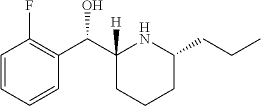 [SMILES: CCC[C@@H]1CCC[C@H]([C@@H](O)c2ccccc2F)N1] | (S)-[(2R,6R)-6-propyl- 2-piperidyl](o- fluorophenyl)methanol | + | − |
| 108 | 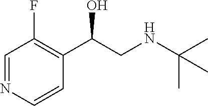 | (R)-2-(tert-butylamino)- 1-(3-fluoro-4-pyridyl)- 1-ethanol | + | − |
| 109 | 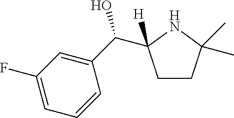 [SMILES: CC1(C)CC[C@H]([C@@H](O)c2cccc(F)c2)N1] | (S)-[(R)-5,5-dimethyl- 2-pyrrolidinyl](m- fluorophenyl)methanol | + | − |
| 110 | 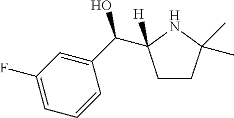 [SMILES: CC1(C)CC[C@H]([C@H](O)c2cccc(F)c2)N1] | (R)-[(R)-5,5-dimethyl- 2-pyrrolidinyl]](m- fluorophenyl)methanol | +++ | − |
| 111 | 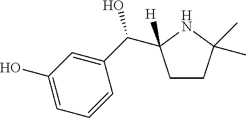 [SMILES: CC1(C)CC[C@H]([C@@H](O)c2cccc(O)c2)N1] | m-{(S)-[(R)-5,5- dimethyl-2- pyrrolidinyl] hydroxymethyl}phenol | ++ | − |
| 112 | 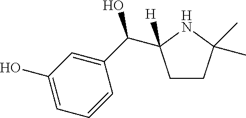 [SMILES: CC1(C)CC[C@H]([C@H](O)c2cccc(O)c2)N1] | m-{(R)-[(R)-5,5- dimethyl-2- pyrrolidinyl] hydroxymethyl}phenol | +++ | − |
| 113 | 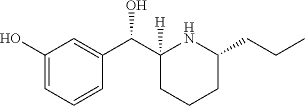 [SMILES: CCC[C@@H]1CCC[C@@H]([C@@H](O)c2cccc(O)c2)N1] | (S)-[(2S,6R)-6-propyl- 2-piperidyl]](m- hydroxyphenyl)methanol | ++ | − |
| 114 | 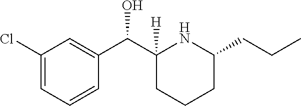 [SMILES: CCC[C@@H]1CCC[C@@H]([C@@H](O)c2cccc(Cl)c2)N1] | (S)-[(2S,6R)-6-propyl- 2-piperidyl]](m- chlorophenyl)methanol | + | − |
| 115 | 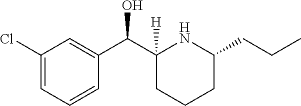 [SMILES: CCC[C@@H]1CCC[C@@H]([C@H](O)c2cccc(Cl)c2)N1] | (R)-[(2S,6R)-6-propyl- 2-piperidyl]](m- chlorophenyl)methanol | +++ | − |
| 116 | 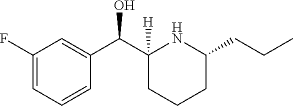 [SMILES: CCC[C@@H]1CCC[C@@H]([C@H](O)c2cccc(F)c2)N1] | (R)-[(2S,6R)-6-propyl- 2-piperidyl]](m- fluorophenyl)methanol | + | − |
| 117 | 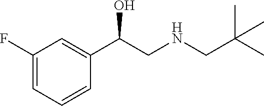 [SMILES: CC(C)(C)CNC[C@H](O)c1cccc(F)c1] | (R)-1-(m-fluorophenyl)- 2-(neopentylamino)-1- ethanol | + | − |
| 118 | 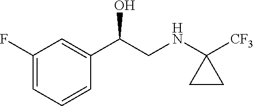 [SMILES: O[C@@H](CNC1(C(F)(F)F)CC1)c1cccc(F)c1] | (R)-1-(m-fluorophenyl)- 2-[1- (trifluoromethyl) cyclopropylamino]- 1-ethanol | ++ | − |
| 119 | 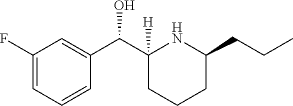 [SMILES: CCC[C@H]1CCC[C@@H]([C@@H](O)c2cccc(F)c2)N1] | (S)-[(2S,6S)-6-propyl- 2-piperidyl]](m- fluorophenyl)methanol | + | − |
| 120 | 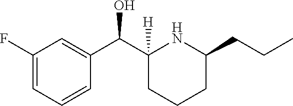 [SMILES: CCC[C@H]1CCC[C@@H]([C@H](O)c2cccc(F)c2)N1] | (R)-[(2S,6S)-6-propyl- 2-piperidyl]](m- fluorophenyl)methanol | ++ | − |
| 121 | 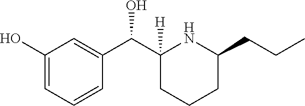 [SMILES: CCC[C@H]1CCC[C@@H]([C@@H](O)c2cccc(O)c2)N1] | (S)-[(2S,6S)-6-propyl- 2-piperidyl]](m- hydroxyphenyl) methanol | +++ | − |
| 122 | 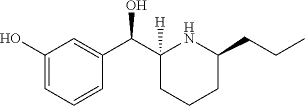 [SMILES: CCC[C@H]1CCC[C@@H]([C@H](O)c2cccc(O)c2)N1] | (R)-[(2S,6S)-6-propyl- 2-piperidyl]](m- hydroxyphenyl) methanol | ++ | − |
| 123 | 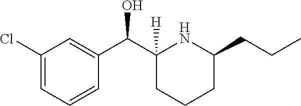 [SMILES: CCC[C@H]1CCC[C@@H]([C@H](O)c2cccc(Cl)c2)N1] | (R)-[(2S,6S)-6-propyl- 2-piperidyl]](m- chlorophenyl)methanol | + | − |
| 124 | 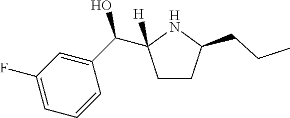 [SMILES: CCC[C@H]1CC[C@H]([C@H](O)c2cccc(F)c2)N1] | (R)-[(2R,5S)-5-propyl- 2-pyrrolidinyl](m- fluorophenyl)methanol | ++ | − |
| 125 | 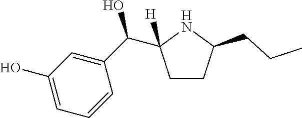 [SMILES: CCC[C@H]1CC[C@H]([C@H](O)c2cccc(O)c2)N1] | m-{(R)-[(2R,5S)-5- propyl-2- pyrrolidinyl] hydroxymethyl}phenol | ++ | − |
| 126 | 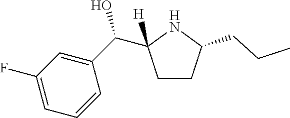 [SMILES: CCC[C@@H]1CC[C@H]([C@@H](O)c2cccc(F)c2)N1] | (S)-[(2R,5R)-5-propyl- 2-pyrrolidinyl]](m- fluorophenyl)methanol | + | − |
| 127 | 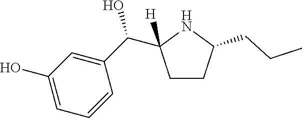 [SMILES: CCC[C@@H]1CC[C@H]([C@@H](O)c2cccc(O)c2)N1] | m-{(S)-[(2R,5R)-5- propyl-2- pyrrolidinyl] hydroxymethyl}phenol | + | − |
| 128 | 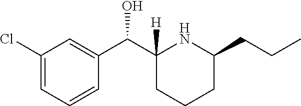 [SMILES: CCC[C@H]1CCC[C@H]([C@@H](O)c2cccc(Cl)c2)N1] | (R)-[(2R,6S)-6-propyl- 2-piperidyl]](m- chlorophenyl)methanol | + | − |
| 129 | 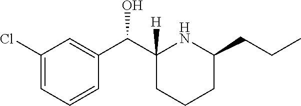 [SMILES: CCC[C@H]1CCC[C@H]([C@@H](O)c2cccc(Cl)c2)N1] | (S)-[(2R,6S)-6-propyl- 2-piperidyl]](m- chlorophenyl)methanol | + | − |
| 130 | 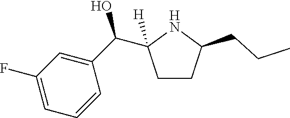 [SMILES: CCC[C@H]1CC[C@@H]([C@H](O)c2cccc(F)c2)N1] | (R)-[(2S,5S)-5-propyl- 2-pyrrolidinyl](m- fluorophenyl)methanol | + | − |
| 131 | 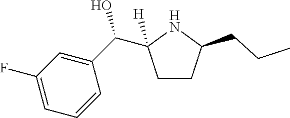 [SMILES: CCC[C@H]1CC[C@@H]([C@@H](O)c2cccc(F)c2)N1] | (S)-[(2S,5S)-5-propyl- 2-pyrrolidinyl](m- fluorophenyl)methanol | ++ | − |
| 132 | 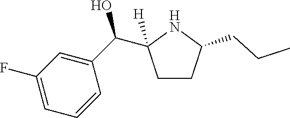 [SMILES: CCC[C@@H]1CC[C@@H]([C@H](O)c2cccc(F)c2)N1] | (R)-[(2S,5R)-5-propyl- 2-pyrrolidinyl](m- fluorophenyl)methanol | + | − |
| 133 | 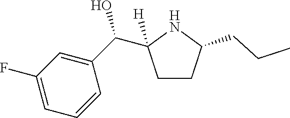 [SMILES: CCC[C@@H]1CC[C@@H]([C@@H](O)c2cccc(F)c2)N1] | (S)-[(2,5R)-5-propyl- 2-pyrrolidinyl](m- fluorophenyl)methanol | ++ | − |
| 134 | 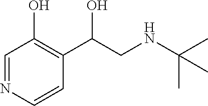 | 4-[2-(tert-butylamino)- 1-hydroxyethyl]-3- pyridinol | +++ | − |
| 135 | 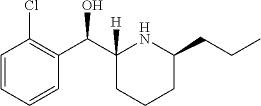 [SMILES: CCC[C@H]1CCC[C@H]([C@H](O)c2ccccc2Cl)N1] | (R)-[(2R,6S)-6-propyl- 2-piperidyl](o- chlorophenyl)methanol | + | − |
| 136 | 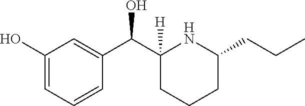 [SMILES: CCC[C@@H]1CCC[C@@H]([C@H](O)c2cccc(O)c2)N1] | (R)-[(2S,6R)-6-propyl- 2-piperidyl]](m- hydroxyphenyl)methanol | +++ | − |
| 137 | 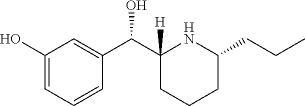 [SMILES: CCC[C@@H]1CCC[C@H]([C@@H](O)c2cccc(O)c2)N1] | (S)-[(2R,6R)-6-propyl- 2-piperidyl](m- hydroxyphenyl)methanol | + | − |
| 138 | 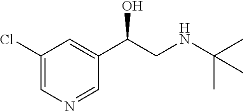 | (R)-2-(tert-butylamino)- 1-(5-chloro-3-pyridyl)- 1-ethanol | +++ | +++ |
| 139 | 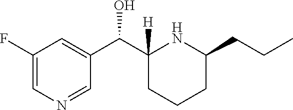 [SMILES: CCC[C@H]1CCC[C@H]([C@@H](O)c2cncc(F)c2)N1] | (S)-[(2R,6S)-6-propyl- 2-piperidyl]](5-fluoro-3- pyridyl)methanol | − | − |
| 140 | 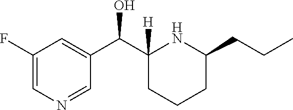 [SMILES: CCC[C@H]1CCC[C@H]([C@H](O)c2cncc(F)c2)N1] | (R)-[(2R,6S)-6-propyl- 2-piperidyl]](5-fluoro-3- pyridyl)methanol | − | − |
| 141 | 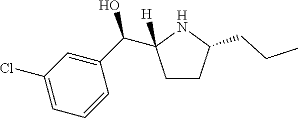 [SMILES: CCC[C@@H]1CC[C@H]([C@H](O)c2cccc(Cl)c2)N1] | (R)-[(2R,5R)-5-propyl- 2-pyrrolidinyl](m- chlorophenyl)methanol | ++ | − |
| 142 | 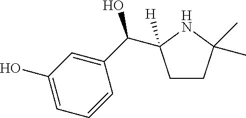 [SMILES: CC1(C)CC[C@@H]([C@H](O)c2cccc(O)c2)N1] | m-{(R)-[(S)-5,5- dimethyl-2- pyrrolidinyl] hydroxymethyl}phenol | + | − |
| 143 | 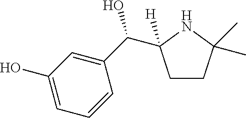 [SMILES: CC1(C)CC[C@@H]([C@@H](O)c2cccc(O)c2)N1] | m-{(S)-[(S)-5,5- dimethyl-2- pyrrolidinyl] ]hydroxymethyl}phenol | − | − |
| 144 | 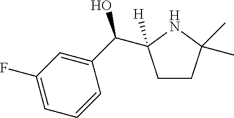 [SMILES: CC1(C)CC[C@@H]([C@H](O)c2cccc(F)c2)N1] | (R)-[(S)-5,5-dimethyl- 2-pyrrolidinyl]](m- fluorophenyl)methanol | − | − |
| 145 | 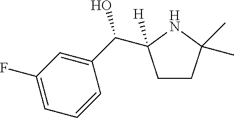 [SMILES: CC1(C)CC[C@@H]([C@@H](O)c2cccc(F)c2)N1] | (S)-[(S)-5,5-dimethyl- 2-pyrrolidinyl](m- fluorophenyl)methanol | + | − |
| 146 | 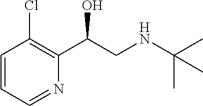 | (S)-2-(tert-butylamino)- 1-(3-chloro-2-pyridyl)- 1-ethanol | + | − |
| 147 | 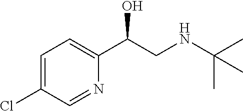 | (S)-2-(tert-butylamino)- 1-(5-chloro-2-pyridyl)- 1-ethanol | + | − |
| 148 | 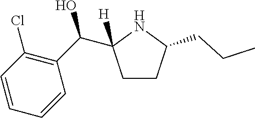 [SMILES: CCC[C@@H]1CC[C@H]([C@H](O)c2ccccc2Cl)N1] | (R)-[(2R,5R)-5-propyl- 2-pyrrolidinyl](o- chlorophenyl)methanol | +++ | − |
| 149 | 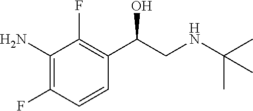 [SMILES: CC(C)(C)NC[C@H](O)c1ccc(F)c(N)c1F] | (R)-1-(3-amino-2,4- difluorophenyl)-2-(tert- butylamino)-1-ethanol | +++ | + |
| 150 | 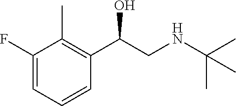 [SMILES: Cc1c(F)cccc1[C@@H](O)CNC(C)(C)C] | (R)-2-(tert-butylamino)- 1-(3-fluoro-2-tolyl)-1- ethanol | +++ | + |
| 151 | 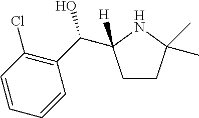 [SMILES: CC1(C)CC[C@H]([C@@H](O)c2ccccc2Cl)N1] | (S)-[(R)-5,5-dimethyl- 2-pyrrolidinyl]](o- chlorophenyl)methanol | − | − |
| 152 | 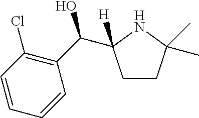 [SMILES: CC1(C)CC[C@H]([C@H](O)c2ccccc2Cl)N1] | (R)-[(R)-5,5-dimethyl- 2-pyrrolidinyl](o- chlorophenyl)methanol | +++ | − |
| 153 | 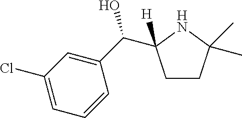 [SMILES: CC1(C)CC[C@H]([C@@H](O)c2cccc(Cl)c2)N1] | (S)-[(R)-5,5-dimethyl- 2-pyrrolidinyl](m- chlorophenyl)methanol | + | − |
| 154 | 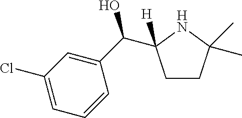 [SMILES: CC1(C)CC[C@H]([C@H](O)c2cccc(Cl)c2)N1] | (R)-[(R)-5,5-dimethyl- 2-pyrrolidinyl](m- chlorophenyl)methanol | +++ | − |
| 155 | 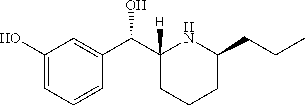 [SMILES: CCC[C@H]1CCC[C@H]([C@@H](O)c2cccc(O)c2)N1] | (S)-[(2R,6S)-6-propyl- 2-piperidyl]](m- hydroxyphenyl) methanol | − | − |
| 156 | 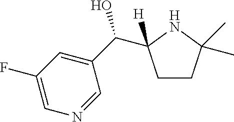 [SMILES: CC1(C)CC[C@H]([C@@H](O)c2cncc(F)c2)N1] | (S)-[(R)-5,5-dimethyl- 2-pyrrolidinyl]](5-fluoro- 3-pyridyl)methanol | − | − |
| 157 | 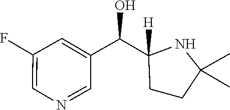 [SMILES: CC1(C)CC[C@H]([C@H](O)c2cncc(F)c2)N1] | (R)-[(R)-5,5-dimethyl- 2-pyrrolidinyl](5-fluoro- 3-pyridyl)methanol | +++ | − |
| 158 | 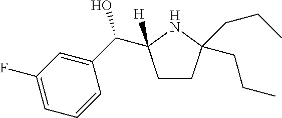 [SMILES: CCCC1(CCC)CC[C@H]([C@@H](O)c2cccc(F)c2)N1] | (S)-[(R)-5,5-dipropyl-2- pyrrolidinyl](m- fluorophenyl)methanol | − | − |
| 159 | 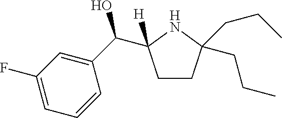 [SMILES: CCCC1(CCC)CC[C@H]([C@H](O)c2cccc(F)c2)N1] | (R)-[(R)-5,5-dipropyl-2- pyrrolidinyl]](m- fluorophenyl)methanol | ++ | − |
| 160 | 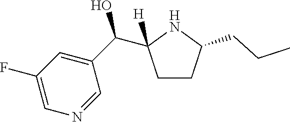 [SMILES: CCC[C@@H]1CC[C@H]([C@H](O)c2cncc(F)c2)N1] | (R)-[(2R,5R)-5-propyl- 2-pyrrolidinyl](5-fluoro- 3-pyridyl)methanol | +++ | − |
| 161 | 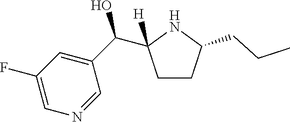 [SMILES: CCC[C@@H]1CC[C@H]([C@H](O)c2cncc(F)c2)N1] | (S)-[(2R,5R)-5-propyl- 2-pyrrolidinyl]](5-fluoro- 3-pyridyl)methanol | + | − |
| 162 | 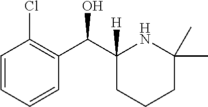 [SMILES: CC1(C)CCC[C@H]([C@H](O)c2ccccc2Cl)N1] | (R)-[(R)-6,6-dimethyl- 2-piperidyl](o- chlorophenyl)methanol | + | − |
| 163 | 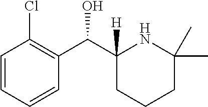 [SMILES: CC1(C)CCC[C@H]([C@@H](O)c2ccccc2Cl)N1] | (S)-[(R)-6,6-dimethyl- 2-piperidyl](o- chlorophenyl)methanol | +++ | − |
| 164 | 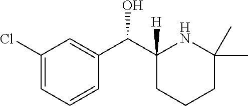 [SMILES: CC1(C)CCC[C@H]([C@@H](O)c2cccc(Cl)c2)N1] | (S)-[(R)-6,6-dimethyl- 2-piperidyl]](m- chlorophenyl)methanol | ++ | − |
| 165 | 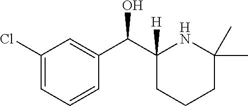 [SMILES: CC1(C)CCC[C@H]([C@H](O)c2cccc(Cl)c2)N1] | (R)-[(R)-6,6-dimethyl- 2-piperidyl]](m- chlorophenyl)methanol | + | − |
| 166 | 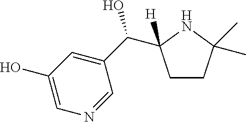 [SMILES: CC1(C)CC[C@H]([C@@H](O)c2cncc(O)c2)N1] | 5-{(S)-[(R)-5,5- dimethyl-2- pyrrolidinyl] hydroxymethyl}-3- pyridinol | + | − |
| 167 | 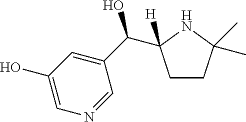 [SMILES: CC1(C)CC[C@H]([C@H](O)c2cncc(O)c2)N1] | 5-{(R)-[(R)-5,5- dimethyl-2- pyrrolidinyl] hydroxymethyl}- 3-pyridinol | ++ | − |
| 168 | 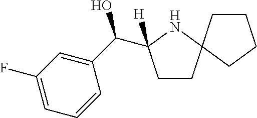 [SMILES: O[C@H](c1cccc(F)c1)[C@H]1CCC2(CCCC2)N1] | (R)-{(R)-1-aza-2- spiro[[4.4]nonyl}(m- fluorophenyl)methanol | ++ | + |
| 169 | 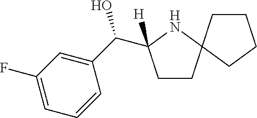 [SMILES: O[C@@H](c1cccc(F)c1)[C@H]1CCC2(CCCC2)N1] | (S)-{(R)-1-aza-2- spiro[[4.4]nonyl}(m- fluorophenyl)methanol | − | − |
| 170 | 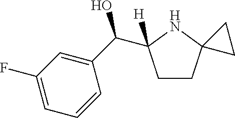 [SMILES: O[C@H](c1cccc(F)c1)[C@H]1CCC2(CC2)N1] | (R)-{(R)-4-aza-5- spiro[2.4]heptyl}(m- fluorophenyl)methanol | ++ | − |
| 171 | 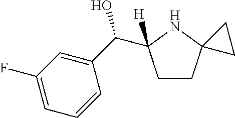 [SMILES: O[C@@H](c1cccc(F)c1)[C@H]1CCC2(CC2)N1] | (S)-{(R)-4-aza-5- spiro[[2.4]heptyl}(m- fluorophenyl)methanol | + | − |
| 172 | 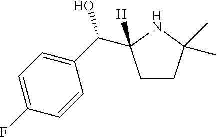 [SMILES: CC1(C)CC[C@H]([C@@H](O)c2ccc(F)cc2)N1] | (S)-[(R)-5,5-dimethyl- 2-pyrrolidinyl]](p- fluorophenyl)methanol | − | − |
| 173 | 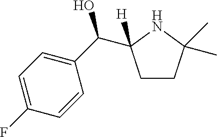 [SMILES: CC1(C)CC[C@H]([C@H](O)c2ccc(F)cc2)N1] | (R)-[(R)-5,5-dimethyl- 2-pyrrolidinyl]](p- fluorophenyl)methanol | ++ | − |
| 174 | 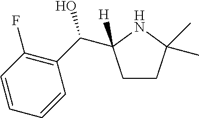 [SMILES: CC1(C)CC[C@H]([C@@H](O)c2ccccc2F)N1] | (S)-[(R)-5,5-dimethyl- 2-pyrrolidinyl](o- fluorophenyl)methanol | − | − |
| 175 | 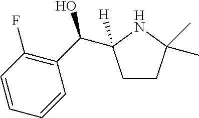 [SMILES: CC1(C)CC[C@@H]([C@H](O)c2ccccc2F)N1] | (R)-[(R)-5,5-dimethyl- 2-pyrrolidinyl](o- fluorophenyl)methanol | +++ | + |
| 176 | 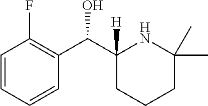 [SMILES: CC1(C)CCC[C@H]([C@@H](O)c2ccccc2F)N1] | (S)-[(R)-6,6-dimethyl- 2-piperidyl](o- fluorophenyl)methanol | +++ | + |
| 177 | 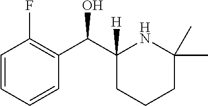 [SMILES: CC1(C)CCC[C@H]([C@H](O)c2ccccc2F)N1] | (R)-[(R)-6,6-dimethyl- 2-piperidyl](o- fluorophenyl)methanol | ++ | − |
| 178 | 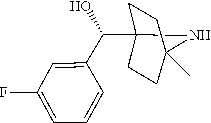 [SMILES: CC12CCC([C@@H](O)c3cccc(F)c3)(CC1)N2] | (S)-(m-fluorophenyl)(4- methyl-7- azabicyclo[[2.2.1]]hept- 1-yl)methanol | + | − |
| 179 | 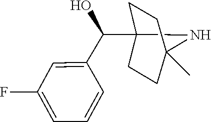 [SMILES: CC12CCC([C@H](O)c3cccc(F)c3)(CC1)N2] | (R)-(m-fluorophenyl)(4- methyl-7- azabicyclo[[2.2.1]]hept- 1-yl)methanol | ++ | − |
| 180 | 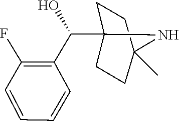 [SMILES: CC12CCC([C@@H](O)c3ccccc3F)(CC1)N2] | (S)-(o-fluorophenyl)(4- methyl-7- azabicyclo[[2.2.1]]hept- 1-yl)methanol | +++ | − |
| 181 | 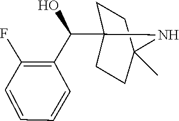 [SMILES: CC12CCC([C@H](O)c3ccccc3F)(CC1)N2] | (R)-(o-fluorophenyl)(4- methyl-7- azabicyclo[[2.2.1]]hept- 1-yl)methanol | +++ | − |
| 182 | 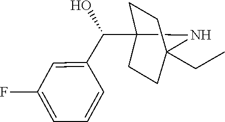 [SMILES: CCC12CCC([C@@H](O)c3cccc(F)c3)(CC1)N2] | (S)-(m-fluorophenyl)(4- propyl-7- azabicyclo[[2.2.1]]hept- 1-yl)methanol | − | − |
| 183 | 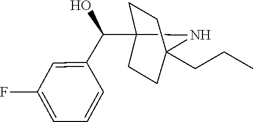 [SMILES: CCCC12CCC([C@H](O)c3cccc(F)c3)(CC1)N2] | (R)-(m-fluorophenyl)(4- propyl-7- azabicyclo[[2.2.1]]hept- 1-yl)methanol | ++ | + |
| 184 | 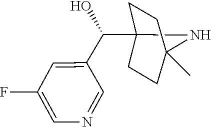 [SMILES: CC12CCC([C@@H](O)c3cncc(F)c3)(CC1)N2] | (S)-(5-fluoro-3- pyridyl)(4-methyl-7- azabicyclo[[2.2.1]]hept- 1-yl)methanol | ++ | − |
| 185 | 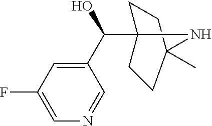 [SMILES: CC12CCC([C@H](O)c3cncc(F)c3)(CC1)N2] | (R)-(5-fluoro-3- pyridyl)(4-methyl-7- azabicyclo[[2.2.1]]hept- 1-yl)methanol | +++ | − |
| 186 | 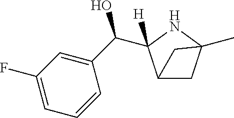 [SMILES: CC12CC(C1)[C@H]([C@H](O)c1cccc(F)c1)N2] | (R)-{(R)-1-methyl-2- azabicyclo[[2.1.1]hex-3- yl}(m- fluorophenyl)methanol | +++ | ++ |
| 187 | 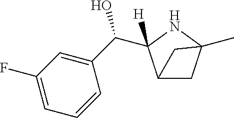 [SMILES: CC12CC(C1)[C@H]([C@@H](O)c1cccc(F)c1)N2] | (S)-{(R)-1-methyl-2- azabicyclo[[2.1.1]hex-3- yl}(m- fluorophenyl)methanol | + | − |
| 188 | 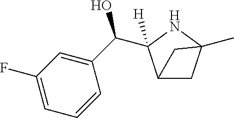 [SMILES: CC12CC(C1)[C@@H]([C@H](O)c1cccc(F)c1)N2] | (R)-{(S)-1-methyl-2- azabicyclo[[2.1.1]]hex-3- yl}(m- fluorophenyl)methanol | − | − |
| 189 | 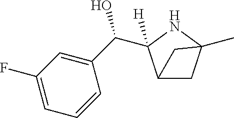 [SMILES: CC12CC(C1)[C@@H]([C@@H](O)c1cccc(F)c1)N2] | (S)-{(S)-1-methyl-2- azabicyclo[2.1.1]]hex-3- yl}(m- fluorophenyl)methanol | ++ | − |
| 190 | 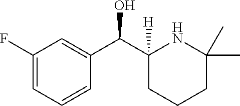 [SMILES: CC1(C)CCC[C@@H]([C@H](O)c2cccc(F)c2)N1] | (R)-[(S)-6,6-dimethyl- 2-piperidyl]](m- fluorophenyl)methanol | +++ | − |
| 191 | 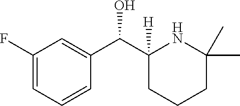 [SMILES: CC1(C)CCC[C@@H]([C@@H](O)c2cccc(F)c2)N1] | (S)-[(S)-6,6-dimethyl- 2-piperidyl]](m- fluorophenyl)methanol | + | − |
| 192 | 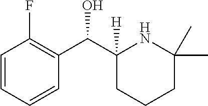 [SMILES: CC1(C)CCC[C@@H]([C@@H](O)c2ccccc2F)N1] | (S)-[(S)-6,6-dimethyl- 2-piperidyl](o- fluorophenyl)methanol | + | − |
| 193 | 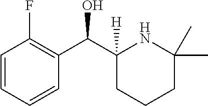 [SMILES: CC1(C)CCC[C@@H]([C@H](O)c2ccccc2F)N1] | (R)-[(S)-6,6-dimethyl- 2-piperidyl]](o- fluorophenyl)methanol | +++ | + |
| 194 | 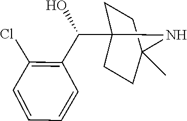 [SMILES: CC12CCC([C@@H](O)c3ccccc3Cl)(CC1)N2] | (S)-(o-chlorophenyl)(4- methyl-7- azabicyclo[[2.2.1]]hept- 1-yl)methanol | +++ | − |
| 195 | 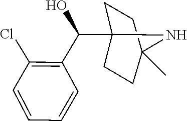 [SMILES: CC12CCC([C@H](O)c3ccccc3Cl)(CC1)N2] | (R)-(o-chlorophenyl)(4- methyl-7- azabicyclo[[2.2.1]]hept- 1-yl)methanol | +++ | − |
| 196 | 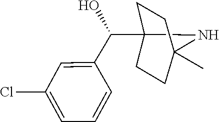 [SMILES: CC12CCC([C@@H](O)c3cccc(Cl)c3)(CC1)N2] | (S)-(m-chlorophenyl)(4- methyl-7- azabicyclo[[2.2.1]]hept- 1-yl)methanol | + | − |
| 197 | 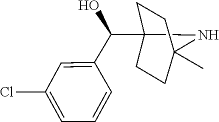 [SMILES: CC12CCC([C@H](O)c3cccc(Cl)c3)(CC1)N2] | (R)-(m-chlorophenyl)(4- methyl-7- azabicyclo[[2.2.1]]hept- 1-yl)methanol | +++ | − |
| 198 | 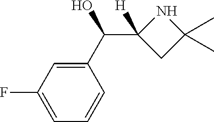 | (R)-[(R)-4,4-dimethyl- 2-azetidinyl](m- fluorophenyl)methanol | +++ | − |
| 199 | 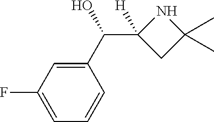 | (S)-[(S)-4,4-dimethyl- 2-azetidinyl]](m- fluorophenyl)methanol | + | − |
| 200 | 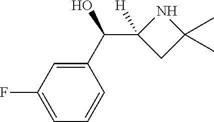 | (R)-[(S)-4,4-dimethyl- 2-azetidinyl](m- fluorophenyl)methanol | − | − |
| 201 | 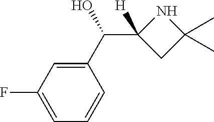 | (S)-[(R)-4,4-dimethyl- 2-azetidinyl]](m- fluorophenyl)methanol | − | − |
| 202 | 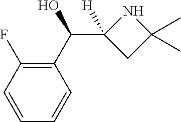 | (R)-[(S)-4,4-dimethyl- 2-azetidinyl](o- fluorophenyl)methanol | − | − |
| 203 | 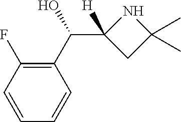 | (S)-[(R)-4,4-dimethyl- 2-azetidinyl](o- fluorophenyl)methanol | + | − |
| 204 | 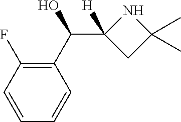 | (R)-[(R)-4,4-dimethyl- 2-azetidinyl](o- fluorophenyl)methanol | +++ | + |
| 205 | 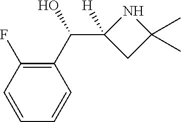 | (S)-[(S)-4,4-dimethyl- 2-azetidinyl](o- fluorophenyl)methanol | + | − |
| 206 | 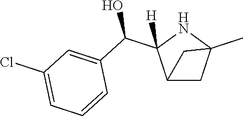 [SMILES: CC12CC(C1)[C@H]([C@H](O)c1cccc(Cl)c1)N2] | (R)-{(R)-1-methyl-2- azabicyclo[2.1.1]]hex-3- yl}(m- chlorophenyl)methanol | +++ | − |
| 207 | 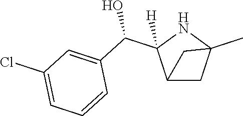 [SMILES: CC12CC(C1)[C@@H]([C@@H](O)c1cccc(Cl)c1)N2] | (S)-{(S)-1-methyl-2- azabicyclo[[2.1.1]hex-3- yl}(m- chlorophenyl)methanol | + | − |
| 208 | 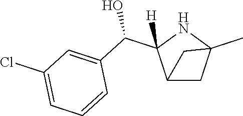 [SMILES: CC12CC(C1)[C@H]([C@@H](O)c1cccc(Cl)c1)N2] | (S)-{(R)-1-methyl-2- azabicyclo[2.1.1]]hex-3- yl}(m- chlorophenyl)methanol | ++ | − |
| 209 | 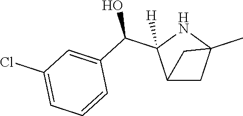 [SMILES: CC12CC(C1)[C@@H]([C@H](O)c1cccc(Cl)c1)N2] | (R)-{(S)-1-methyl-2- azabicyclo[2.1.1]]hex-3- yl}(m- chlorophenyl)methanol | + | − |
| 210 | 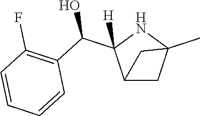 [SMILES: CC12CC(C1)[C@H]([C@H](O)c1ccccc1F)N2] | (R)-{(R)-1-methyl-2- azabicyclo[2.1.1]]hex-3- yl}(o- fluorophenyl)methanol | +++ | +++ |
| 211 | 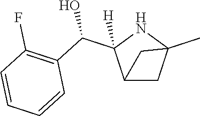 [SMILES: CC12CC(C1)[C@@H]([C@@H](O)c1ccccc1F)N2] | (S)-{(S)-1-methyl-2- azabicyclo[[2.1.1]]hex-3- yl}(o- fluorophenyl)methanol | +++ | − |
| 212 | 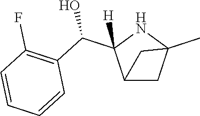 [SMILES: CC12CC(C1)[C@H]([C@@H](O)c1ccccc1F)N2] | (S)-{(R)-1-methyl-2- azabicyclo[[2.1.1]hex-3- yl}(o- fluorophenyl)methanol | +++ | − |
| 213 | 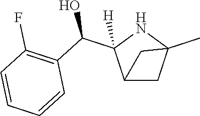 [SMILES: CC12CC(C1)[C@@H]([C@H](O)c1ccccc1F)N2] | (R)-{(S)-1-methyl-2- azabicyclo[[2.1.1]hex-3- yl}(o- fluorophenyl)methanol | +++ | − |
| 214 | 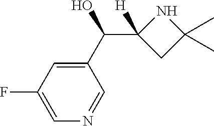 | (R)-[(R)-4,4-dimethyl- 2-azetidinyl]](5-fluoro-3- pyridyl)methanol | +++ | − |
| 215 | 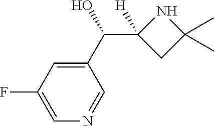 | (S)-[(S)-4,4-dimethyl- 2-azetidinyl]](5-fluoro-3- pyridyl)methanol | + | − |
| 216 | 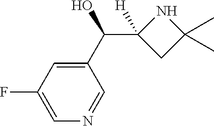 | (R)-[(S)-4,4-dimethyl- 2-azetidinyl]](5-fluoro-3- pyridyl)methanol | + | − |
| 217 | 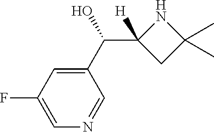 | (S)-[(R)-4,4-dimethyl- 2-azetidinyl]](5-fluoro-3- pyridyl)methanol | + | − |
| 218 | 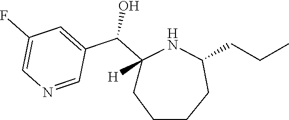 [SMILES: CCC[C@@H]1CCCC[C@H]([C@@H](O)c2cncc(F)c2)N1] | (S)-[(2R,7R)-7-propyl- 2-azepanyl](5-fluoro-3- pyridyl)methanol | − | − |
| 219 | 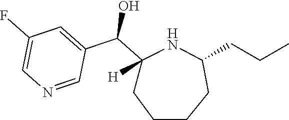 [SMILES: CCC[C@@H]1CCCC[C@H]([C@H](O)c2cncc(F)c2)N1] | (R)-[(2R,7R)-7-propyl- 2-azepanyl](5-fluoro-3- pyridyl)methanol | − | − |
| 220 | 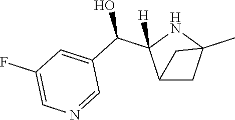 [SMILES: CC12CC(C1)[C@H]([C@H](O)c1cncc(F)c1)N2] | (R)-{(R)-1-methyl-2- azabicyclo[[2.1.1]]hex-3- yl}(5-fluoro-3- pyridyl)methanol | +++ | − |
| 221 | 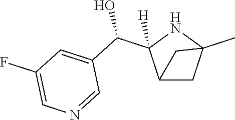 [SMILES: CC12CC(C1)[C@@H]([C@@H](O)c1cncc(F)c1)N2] | (S)-{(S)-1-methyl-2- azabicyclo[[2.1.1]]hex-3- yl}(5-fluoro-3- pyridyl)methanol | ++ | − |
| 222 | 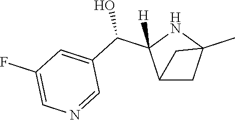 [SMILES: CC12CC(C1)[C@H]([C@@H](O)c1cncc(F)c1)N2] | (S)-{(R)-1-methyl-2- azabicyclo[[2.1.1]]hex-3- yl}(5-fluoro-3- pyridyl)methanol | − | − |
| 223 | 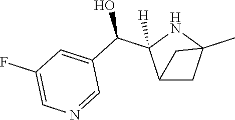 [SMILES: CC12CC(C1)[C@@H]([C@H](O)c1cncc(F)c1)N2] | (R)-{(S)-1-methyl-2- azabicyclo[[2.1.1]]hex-3- yl}(5-fluoro-3- pyridyl)methanol | + | − |
| 224 | 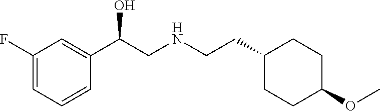 [SMILES: CO[C@H]1CC[C@H](CCNC[C@H](O)c2cccc(F)c2)CC1] | (R)-1-(m-fluorophenyl)- 2-{2-[(1r,4R)-4- methoxycyclohexyl] ethylamino}-1-ethanol | + | − |
| 225 | 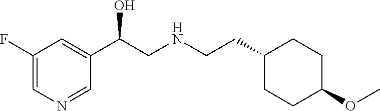 [SMILES: CO[C@H]1CC[C@H](CCNC[C@H](O)c2cncc(F)c2)CC1] | (R)-1-(5-fluoro-3- pyridyl)-2-{2-[(1r,4R)- 4- methoxycyclohexyl] ethylamino}-1-ethanol | ++ | − |
| 226 | 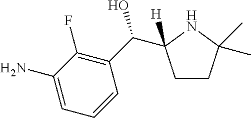 [SMILES: CC1(C)CC[C@H]([C@@H](O)c2cccc(N)c2F)N1] | (S)-[(R)-5,5-dimethyl- 2-pyrrolidinyl](3-amino- 2- fluorophenyl)methanol | + | − |
| 227 | 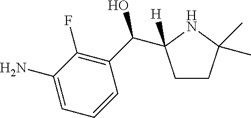 [SMILES: CC1(C)CC[C@H]([C@H](O)c2cccc(N)c2F)N1] | (R)-[(R)-5,5-dimethyl- 2-pyrrolidinyl](3-amino- 2- fluorophenyl)methanol | +++ | − |
| 228 | 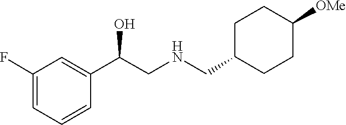 [SMILES: CO[C@H]1CC[C@H](CNC[C@H](O)c2cccc(F)c2)CC1] | (R)-1-(m-fluorophenyl)- 2-({[(1r,4R)-4- methoxycyclohexyl] methyl}amino)-1-ethanol | ++ | − |
| 229 | 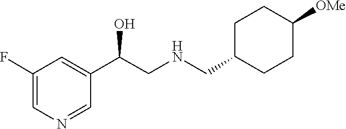 [SMILES: CO[C@H]1CC[C@H](CNC[C@H](O)c2cncc(F)c2)CC1] | (R)-1-(5-fluoro-3- pyridyl)-2-({[(1r,4R)-4- methoxycyclohexyl] methyl}amino)-1-ethanol | ++ | − |
| 230 | 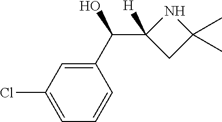 | (R)-[(R)-4,4-dimethyl- 2-azetidinyl]](m- chlorophenyl)methanol | +++ | − |
| 231 | 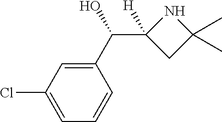 | (S)-[(S)-4,4-dimethyl- 2-azetidinyl]](m- chlorophenyl)methanol | + | − |
| 232 | 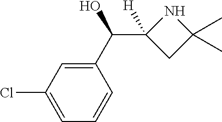 | (R)-[(S)-4,4-dimethyl- 2-azetidinyl]](m- chlorophenyl)methanol | + | − |
| 233 | 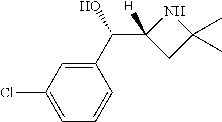 | (S)-[(R)-4,4-dimethyl- 2-azetidinyl](m- chlorophenyl)methanol | − | − |
| 234 | 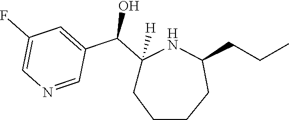 [SMILES: CCC[C@H]1CCCC[C@@H]([C@H](O)c2cncc(F)c2)N1] | (R)-[(2S,7S)-7-propyl- 2-azepanyl](5-fluoro-3- pyridyl)methanol | +++ | − |
| 235 | 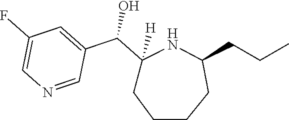 [SMILES: CCC[C@H]1CCCC[C@@H]([C@@H](O)c2cncc(F)c2)N1] | (S)-[(2S,7S)-7-propyl- 2-azepanyl](5-fluoro-3- pyridyl)methanol | − | − |
| 236 | 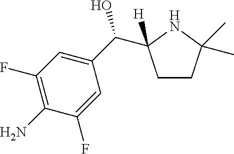 [SMILES: CC1(C)CC[C@H]([C@@H](O)c2cc(F)c(N)c(F)c2)N1] | (S)-[(R)-5,5-dimethyl- 2-pyrrolidinyl]](4-amino- 3,5- difluorophenyl)methanol | +++ | + |
| 237 | 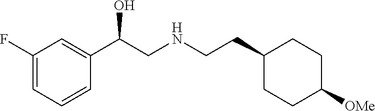 [SMILES: CO[C@H]1CC[C@@H](CCNC[C@H](O)c2cccc(F)c2)CC1] | (R)-1-(m-fluorophenyl)- 2-{2-[(1s,4S)-4- methoxycyclohexyl] ethylamino}-1-ethanol | ++ | − |
| 238 | 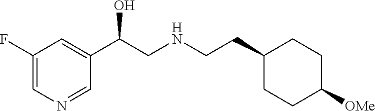 [SMILES: CO[C@H]1CC[C@@H](CCNC[C@H](O)c2cncc(F)c2)CC1] | (R)-1-(5-fluoro-3- pyridyl)-2-{2-[(1s,4S)- 4- methoxycyclohexyl] ethylamino}-1-ethanol | +++ | − |
| 239 | 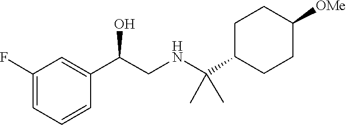 [SMILES: CO[C@H]1CC[C@H](C(C)(C)NC[C@H](O)c2cccc(F)c2)CC1] | (R)-1-(m-fluorophenyl)- 2-{1-methyl-1- [(1r,4R)-4- methoxycyclohexyl]ethy lamino}-1-ethanol | +++ | − |
| 240 | 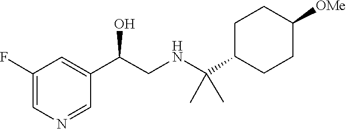 [SMILES: CO[C@H]1CC[C@H](C(C)(C)NC[C@H](O)c2cncc(F)c2)CC1] | (R)-1-(5-fluoro-3- pyridyl)-2-{1-methyl-1- [(1r,4R)-4- methoxycyclohexyl]ethy lamino}-1-ethanol | +++ | − |
| 241 | 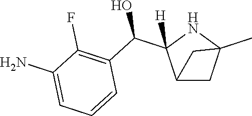 [SMILES: CC12CC(C1)[C@H]([C@H](O)c1cccc(N)c1F)N2] | (R)-{(R)-1-methyl-2- azabicyclo[2.1.1]hex-3- yl}(3-amino-2- fluorophenyl)methanol | +++ | − |
| 242 | 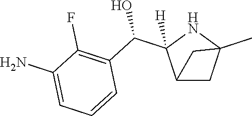 [SMILES: CC12CC(C1)[C@@H]([C@@H](O)c1cccc(N)c1F)N2] | (S)-{(S)-1-methyl-2- azabicyclo[[2.1.1]]hex-3- yl}(3-amino-2- fluorophenyl)methanol | ++ | − |
| 243 | 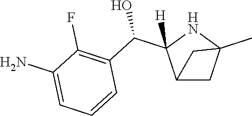 [SMILES: CC12CC(C1)[C@H]([C@@H](O)c1cccc(N)c1F)N2] | (S)-{(R)-1-methyl-2- azabicyclo[[2.1.1]]hex-3- yl}(3-amino-2- fluorophenyl)methanol | ++ | − |
| 244 | 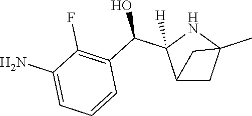 [SMILES: CC12CC(C1)[C@@H]([C@H](O)c1cccc(N)c1F)N2] | (R)-{(S)-1-methyl-2- azabicyclo[[2.1.1]]hex-3- yl}(3-amino-2- fluorophenyl)methanol | ++ | − |
| 245 | 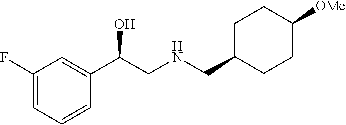 [SMILES: CO[C@H]1CC[C@@H](CNC[C@H](O)c2cccc(F)c2)CC1] | (R)-1-(m-fluorophenyl)- 2-({[(1s,4S)-4- methoxycyclohexyl] methyl}amino)-1-ethanol | ++ | − |
| 246 | 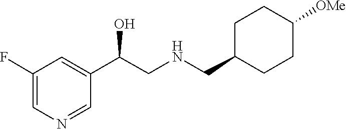 [SMILES: CO[C@H]1CC[C@H](CNC[C@H](O)c2cncc(F)c2)CC1] | (R)-1-(5-fluoro-3- pyridyl)-2-({[(1s,4S)-4- methoxycyclohexyl] methyl}amino)-1-ethanol | ++ | − |
| 247 | 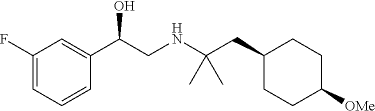 [SMILES: CO[C@H]1CC[C@@H](CC(C)(C)NC[C@H](O)c2cccc(F)c2)CC1] | (R)-2-{1,1-dimethyl-2- [(1s,4S)-4- methoxycyclohexyl] ethylamino}-1-(m- fluorophenyl)-1-ethanol | +++ | − |
| 248 | 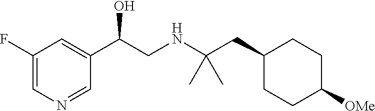 [SMILES: CO[C@H]1CC[C@@H](CC(C)(C)NC[C@H](O)c2cncc(F)c2)CC1] | (R)-2-{1,1-dimethyl-2- [(1s,4S)-4- methoxycyclohexyl] ethylamino}-1-(5- fluoro-3- pyridyl)-1-ethanol | +++ | − |
| 249 | 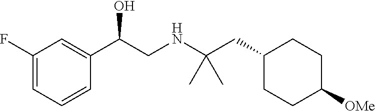 [SMILES: CO[C@H]1CC[C@H](CC(C)(C)NC[C@H](O)c2cccc(F)c2)CC1] | (R)-2-{1,1-dimethyl-2- [(1r,4R)-4- methoxycyclohexyl] ethylamino}-1-(m- fluorophenyl)-1-ethanol | +++ | − |
| 250 | 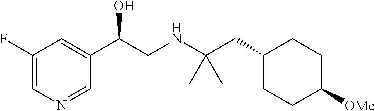 [SMILES: CO[C@H]1CC[C@H](CC(C)(C)NC[C@H](O)c2cncc(F)c2)CC1] | (R)-2-{1,1-dimethyl-2- [(1r,4R)-4- methoxycyclohexyl] ethylamino}-1-(5- fluoro-3- pyridyl)-1-ethanol | ++ | − |
| 251 | 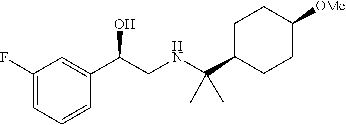 [SMILES: CO[C@H]1CC[C@@H](C(C)(C)NC[C@H](O)c2cccc(F)c2)CC1] | (R)-1-(m-fluorophenyl)- 2-{1-methyl-1- [(1s,4S)-4- methoxycyclohexyl] ethylamino}-1-ethanol | − | − |
| 252 | 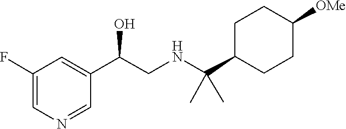 [SMILES: CO[C@H]1CC[C@@H](C(C)(C)NC[C@H](O)c2cncc(F)c2)CC1] | (R)-1-(5-fluoro-3- pyridyl)-2-{1-methyl-1- [(1s,4S)-4- methoxycyclohexyl] ethylamino}-1-ethanol | − | − |
| 253 | 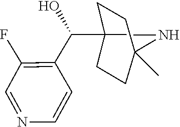 [SMILES: CC12CCC([C@@H](O)c3ccncc3F)(CC1)N2] | (S)-(3-fluoro-4- pyridyl)(4-methyl-7- azabicyclo[[2.2.1]]hept- 1-yl)methanol | − | − |
| 254 | 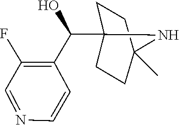 [SMILES: CC12CCC([C@H](O)c3ccncc3F)(CC1)N2] | (R)-(3-fluoro-4- pyridyl)(4-methyl-7- azabicyclo[[2.2.1]]hept- 1-yl)methanol | − | − |
| 255 | 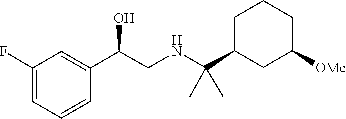 [SMILES: CO[C@@H]1CCC[C@H](C(C)(C)NC[C@H](O)c2cccc(F)c2)C1] | (R)-2-{1-[(1S,3R)-3- methoxycyclohexyl]-1- methylethylamino}-1- (m-fluorophenyl)-1- ethanol | +++ | − |
| 256 | 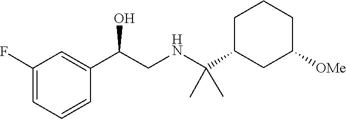 [SMILES: CO[C@H]1CCC[C@@H](C(C)(C)NC[C@H](O)c2cccc(F)c2)C1] | (R)-2-{1-[(1R,3S)-3- methoxycyclohexyl]-1- methylethylamino}-1- (m-fluorophenyl)-1- ethanol | +++ | + |
| 257 | 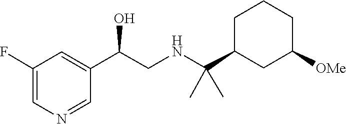 [SMILES: CO[C@@H]1CCC[C@H](C(C)(C)NC[C@H](O)c2cncc(F)c2)C1] | (R)-2-{1-[(1S,3R)-3- methoxycyclohexyl]-1- methylethylamino}-1- (5-fluoro-3-pyridyl)-1- ethanol | ++ | − |
| 258 | 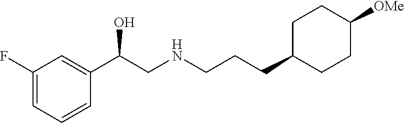 [SMILES: CO[C@H]1CC[C@@H](CCCNC[C@H](O)c2cccc(F)c2)CC1] | (R)-1-(m-fluorophenyl)- 2-{3-[(1s,4R)-4- methoxycyclohexyl] propylamino}-1- ethanol | +++ | ++ |
| 259 | 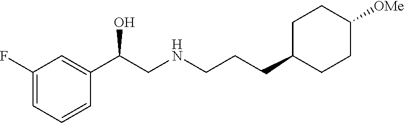 [SMILES: CO[C@H]1CC[C@H](CCCNC[C@H](O)c2cccc(F)c2)CC1] | (R)-1-(m-fluorophenyl)- 2-{3-[(1r,4S)-4- methoxycyclohexyl] propylamino}-1-ethanol | + | − |
| 260 | 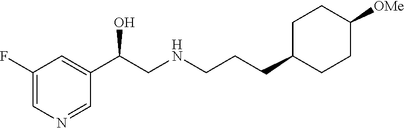 [SMILES: CO[C@H]1CC[C@@H](CCCNC[C@H](O)c2cncc(F)c2)CC1] | (R)-1-(5-fluoro-3- pyridyl)-2-{3-[(1s,4R)- 4- methoxycyclohexyl] propylamino}-1-ethanol | +++ | − |
| 261 | 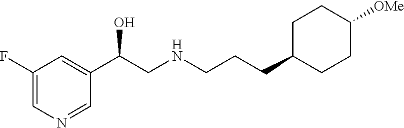 [SMILES: CO[C@H]1CC[C@H](CCCNC[C@H](O)c2cncc(F)c2)CC1] | (R)-1-(5-fluoro-3- pyridyl)-2-{3-[(1r,4S)- 4- methoxycyclohexyl] propylamino}-1-ethanol | − | − |
| 262 | 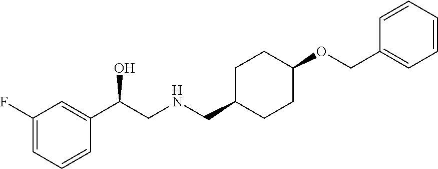 [SMILES: O[C@@H](CNC[C@H]1CC[C@@H](OCc2ccccc2)CC1)c1cccc(F)c1] | (R)-1-(m-fluorophenyl)- 2-({[(1s,4S)-4- (benzyloxy)cyclohexyl] methyl}amino)-1- ethanol | + | − |
| 263 | 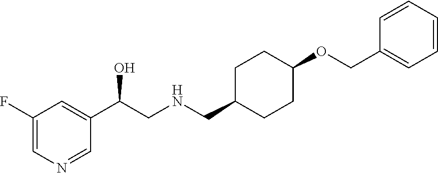 [SMILES: O[C@@H](CNC[C@H]1CC[C@@H](OCc2ccccc2)CC1)c1cncc(F)c1] | (R)-1-(5-fluoro-3- pyridyl)-2-({[(1s,4S)-4- (benzyloxy)cyclohexyl] methyl}amino)-1- ethanol | +++ | − |
| 264 | 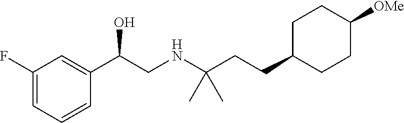 [SMILES: CO[C@H]1CC[C@@H](CCC(C)(C)NC[C@H](O)c2cccc(F)c2)CC1] | (R)-2-{1,1-dimethyl-3- [(1s,4R)-4- methoxycyclohexyl]prop ylamino}-1-(m- fluorophenyl)-1-ethanol | +++ | + |
| 265 | 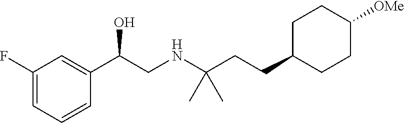 [SMILES: CO[C@H]1CC[C@H](CCC(C)(C)NC[C@H](O)c2cccc(F)c2)CC1] | (R)-2-{1,1-dimethyl-3- [(1r,4S)-4- methoxycyclohexyl] propylamino}-1-(m- fluorophenyl)-1-ethanol | ++ | − |
| 266 | 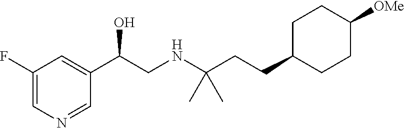 [SMILES: CO[C@H]1CC[C@@H](CCC(C)(C)NC[C@H](O)c2cncc(F)c2)CC1] | (1s,4R)-4- methoxycyclohexyl] propylamino}-1-(5- fluoro-3- pyridyl)-1-ethanol | +++ | − |
| 267 | 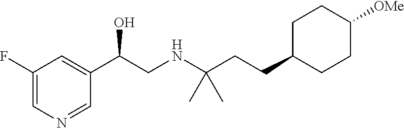 [SMILES: CO[C@H]1CC[C@H](CCC(C)(C)NC[C@H](O)c2cncc(F)c2)CC1] | (R)-2-{1,1-dimethyl-3- [(1r,4S)-4- methoxycyclohexyl] propylamino}-1- (5-fluoro-3- pyridyl)-1-ethanol | + | − |
| 268 | 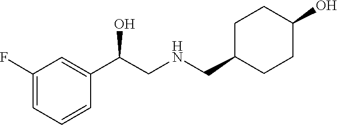 [SMILES: O[C@@H](CNC[C@H]1CC[C@@H](O)CC1)c1cccc(F)c1] | (R)-1-(m-fluorophenyl)- 2-({[(1s,4S)-4- hydroxycyclohexyl] methyl}amino)-1-ethanol | + | − |
| 269 | 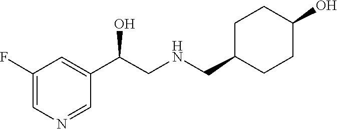 [SMILES: O[C@@H](CNC[C@H]1CC[C@@H](O)CC1)c1cncc(F)c1] | (R)-1-(5-fluoro-3- pyridyl)-2-({[(1s,4S)-4- hydroxycyclohexyl] methyl}amino)-1-ethanol | + | − |
| 270 | 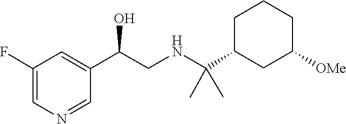 [SMILES: CO[C@H]1CCC[C@@H](C(C)(C)NC[C@H](O)c2cncc(F)c2)C1] | (R)-2-{1-[(1R,3S)-3- methoxycyclohexyl]-1- methylethylamino}-1- (5-fluoro-3-pyridyl)-1- ethanol | +++ | ++ |
| 271 | 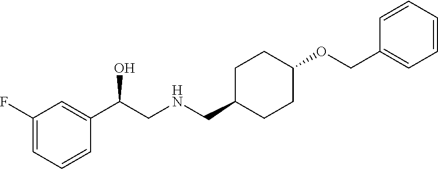 [SMILES: O[C@@H](CNC[C@H]1CC[C@H](OCc2ccccc2)CC1)c1cccc(F)c1] | (R)-1-(m-fluorophenyl)- 2-({[(1r,4R)-4- (benzyloxy)cyclohexyl] methyl}amino)-1- ethanol | + | − |
| 272 | 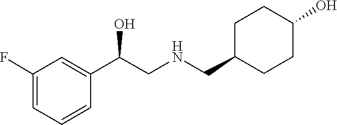 [SMILES: O[C@@H](CNC[C@H]1CC[C@H](O)CC1)c1cccc(F)c1] | (R)-1-(m-fluorophenyl)- 2-({[(1r,4R)-4- hydroxycyclohexyl] methyl}amino)-1-ethanol | + | − |
| 273 | 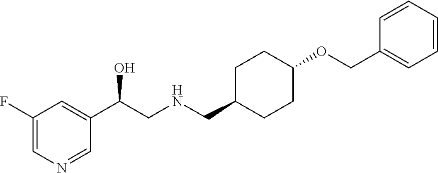 [SMILES: O[C@@H](CNC[C@H]1CC[C@H](OCc2ccccc2)CC1)c1cncc(F)c1] | (R)-1-(5-fluoro-3- pyridyl)-2-({[(1r,4R)-4- (benzyloxy)cyclohexyl] methyl}amino)-1- ethanol | + | − |
| 274 | 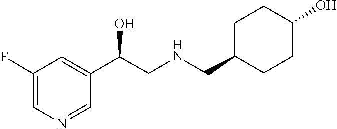 [SMILES: O[C@@H](CNC[C@H]1CC[C@H](O)CC1)c1cncc(F)c1] | (R)-1-(5-fluoro-3- pyridyl)-2-({[(1r,4R)-4- hydroxycyclohexyl] methyl}amino)-1-ethanol | − | − |
| 275 | 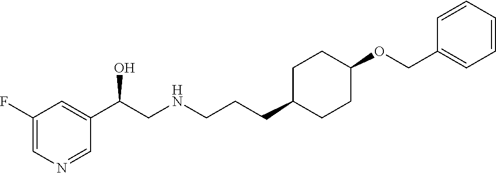 [SMILES: O[C@@H](CNCCC[C@H]1CC[C@@H](OCc2ccccc2)CC1)c1cncc(F)c1] | (R)-1-(5-fluoro-3- pyridyl)-2-{3-[(1s,4R)- 4- (benzyloxy)cyclohexyl] propylamino}-1-ethanol | ++ | − |
| 276 | 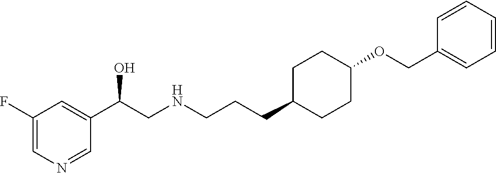 [SMILES: O[C@@H](CNCCC[C@H]1CC[C@H](OCc2ccccc2)CC1)c1cncc(F)c1] | (R)-1-(5-fluoro-3- pyridyl)-2-{3-[(1r,4S)- 4- (benzyloxy)cyclohexyl] propylamino}-1-ethanol | + | − |
| 277 | 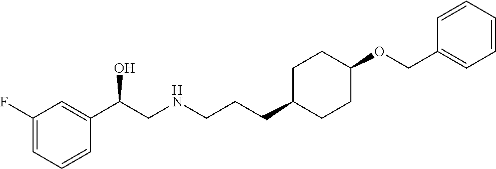 [SMILES: O[C@@H](CNCCC[C@H]1CC[C@@H](OCc2ccccc2)CC1)c1cccc(F)c1] | (R)-1-(m-fluorophenyl)- 2-{3-[(1s,4R)-4- (benzyloxy)cyclohexyl] propylamino}-1-ethanol | + | − |
| 278 | 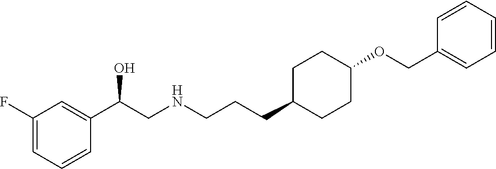 [SMILES: O[C@@H](CNCCC[C@H]1CC[C@H](OCc2ccccc2)CC1)c1cccc(F)c1] | (R)-1-(m-fluorophenyl)- 2-{3-[(1r,4S)-4- (benzyloxy)cyclohexyl] propylamino}-1-ethanol | + | − |
| 279 | 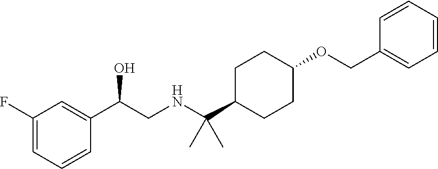 [SMILES: CC(C)(NC[C@H](O)c1cccc(F)c1)[C@H]1CC[C@H](OCc2ccccc2)CC1] | (R)-1-(m-fluorophenyl)- 2-{1-methyl-1- [(1r,4R)-4- (benzyloxy)cyclohexyl] ethylamino}-1-ethanol | ++ | − |
| 280 | 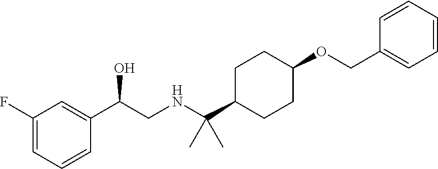 [SMILES: CC(C)(NC[C@H](O)c1cccc(F)c1)[C@H]1CC[C@@H](OCc2ccccc2)CC1] | (R)-1-(m-fluorophenyl)- 2-{1-methyl-1- [(1s,4S)-4- (benzyloxy)cyclohexyl] ethylamino}-1-ethanol | +++ | − |
| 281 | 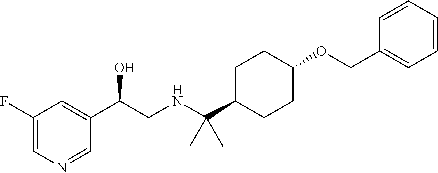 [SMILES: CC(C)(NC[C@H](O)c1cncc(F)c1)[C@H]1CC[C@H](OCc2ccccc2)CC1] | (R)-1-(5-fluoro-3- pyridyl)-2-{1-methyl-1- [(1r,4R)-4- (benzyloxy)cyclohexyl] ethylamino}-1-ethanol | ++ | − |
| 282 | 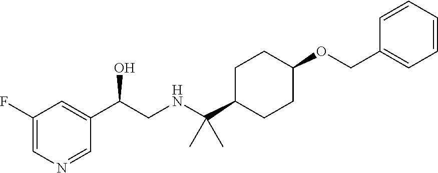 [SMILES: CC(C)(NC[C@H](O)c1cncc(F)c1)[C@H]1CC[C@@H](OCc2ccccc2)CC1] | (R)-1-(5-fluoro-3- pyridyl)-2-{1-methyl-1- [(1s,4S)-4- (benzyloxy)cyclohexyl] ethylamino}-1-ethanol | ++ | − |
| 283 | 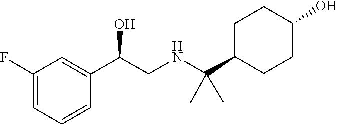 [SMILES: CC(C)(NC[C@H](O)c1cccc(F)c1)[C@H]1CC[C@H](O)CC1] | (R)-1-(m-fluorophenyl)- 2-{1-methyl-1- [(1r,4R)-4- hydroxycyclohexyl]ethyl amino}-1-ethanol | ++ | − |
| 284 | 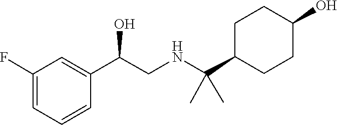 [SMILES: CC(C)(NC[C@H](O)c1cccc(F)c1)[C@H]1CC[C@@H](O)CC1] | (R)-1-(m-fluorophenyl)- 2-{1-methyl-1- [(1s,4S)-4- hydroxycyclohexyl]ethyl amino}-1-ethanol | ++ | − |
| 285 | 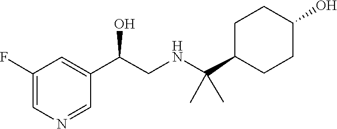 [SMILES: CC(C)(NC[C@H](O)c1cncc(F)c1)[C@H]1CC[C@H](O)CC1] | (R)-1-(5-fluoro-3- pyridyl)-2-{1-methyl-1- [(1r,4R)-4- hydroxycyclohexyl]ethyl amino}-1-ethanol | ++ | − |
| 286 | 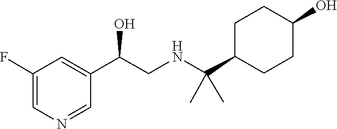 [SMILES: CC(C)(NC[C@H](O)c1cncc(F)c1)[C@H]1CC[C@@H](O)CC1] | (R)-1-(5-fluoro-3- pyridyl)-2-{1-methyl-1- [(1s,4S)-4- hydroxycyclohexyl]ethyl amino}-1-ethanol | + | − |
| 287 | 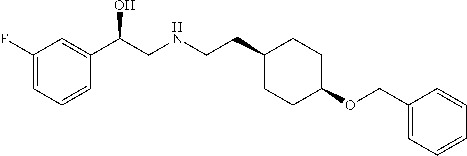 [SMILES: O[C@@H](CNCC[C@H]1CC[C@@H](OCc2ccccc2)CC1)c1cccc(F)c1] | (R)-1-(m-fluorophenyl)- 2-{2-[(1s,4S)-4- (benzyloxy)cyclohexyl] ethylamino}-1-ethanol | ++ | − |
| 288 | 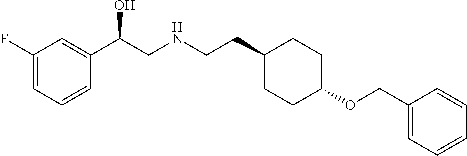 [SMILES: O[C@@H](CNCC[C@H]1CC[C@H](OCc2ccccc2)CC1)c1cccc(F)c1] | (R)-1-(m-fluorophenyl)- 2-{2-[(1r,4R)-4- (benzyloxy)cyclohexyl] ethylamino}-1-ethanol | + | − |
| 289 | 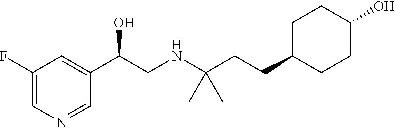 [SMILES: CC(C)(CC[C@H]1CC[C@H](O)CC1)NC[C@H](O)c1cncc(F)c1] | (R)-2-{1,1-dimethyl-3- [(1r,4S)-4- hydroxycyclohexyl] propylamino}-1-(5- fluoro-3- pyridyl)-1-ethanol | + | − |
| 290 | 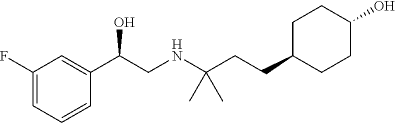 [SMILES: CC(C)(CC[C@H]1CC[C@H](O)CC1)NC[C@H](O)c1cccc(F)c1] | (R)-2-{1,1-dimethyl-3- [(1r,4S)-4- hydroxycyclohexyl] propylamino}-1-(m- fluorophenyl)-1-ethanol | + | − |
| 291 | 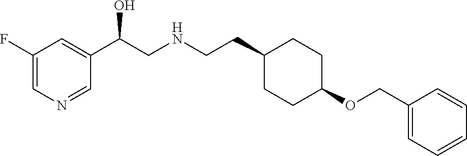 [SMILES: O[C@@H](CNCC[C@H]1CC[C@@H](OCc2ccccc2)CC1)c1cncc(F)c1] | (R)-1-(5-fluoro-3- pyridyl)-2-{2-[(1s,4S)- 4- (benzyloxy)cyclohexyl] ethylamino}-1-ethanol | ++ | − |
| 292 | 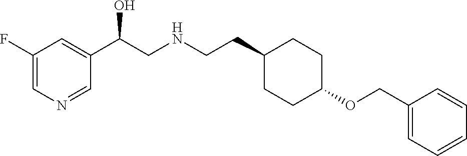 [SMILES: O[C@@H](CNCC[C@H]1CC[C@H](OCc2ccccc2)CC1)c1cncc(F)c1] | (R)-1-(5-fluoro-3- pyridyl)-2-{2-[(1r,4R)- 4- (benzyloxy)cyclohexyl] ethylamino}-1-ethanol | − | − |
| 293 | 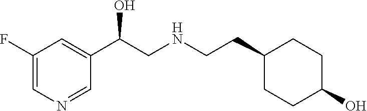 [SMILES: O[C@@H](CNCC[C@H]1CC[C@@H](O)CC1)c1cncc(F)c1] | (R)-1-(5-fluoro-3- pyridyl)-2-{2-[(1s,4S)- 4- hydroxycyclohexyl]ethyl amino}-1-ethanol | + | − |
| 294 | 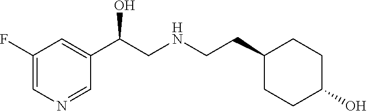 [SMILES: O[C@@H](CNCC[C@H]1CC[C@H](O)CC1)c1cncc(F)c1] | (R)-1-(5-fluoro-3- pyridyl)-2-{2-[(1r,4R)- 4- hydroxycyclohexyl]ethyl amino}-1-ethanol | + | − |
| 295 | 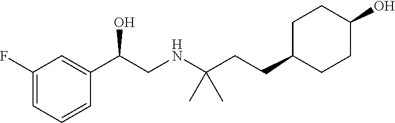 [SMILES: CC(C)(CC[C@H]1CC[C@@H](O)CC1)NC[C@H](O)c1cccc(F)c1] | (R)-2-{1,1-dimethyl-3- [(1s,4R)-4- hydroxycyclohexyl] propylamino}-1-(m- fluorophenyl)-1-ethanol | +++ | − |
| 296 | 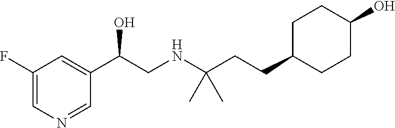 [SMILES: CC(C)(CC[C@H]1CC[C@@H](O)CC1)NC[C@H](O)c1cncc(F)c1] | (R)-2-{1,1-dimethyl-3- [(1s,4R)-4- hydroxycyclohexyl] propylamino}-1-(5- fluoro-3- pyridyl)-1-ethanol | +++ | − |
| 297 | 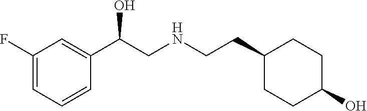 [SMILES: O[C@@H](CNCC[C@H]1CC[C@@H](O)CC1)c1cccc(F)c1] | (R)-1-(m-fluorophenyl)- 2-{2-[(1s,4S)-4- hydroxycyclohexyl]ethyl amino}-1-ethanol | ++ | − |
| 298 | 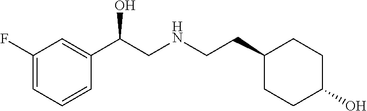 [SMILES: O[C@@H](CNCC[C@H]1CC[C@H](O)CC1)c1cccc(F)c1] | (R)-1-(m-fluorophenyl)- 2-{2-[(1r,4R)-4- hydroxycyclohexyl]ethyl amino}-1-ethanol | + | − |
| 299 | 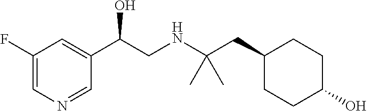 [SMILES: CC(C)(C[C@H]1CC[C@H](O)CC1)NC[C@H](O)c1cncc(F)c1] | (R)-2-{1,1-dimethyl-2- [(1r,4R)-4- hydroxycyclohexyl]ethyl amino}-1-(5-fluoro-3- pyridyl)-1-ethanol | ++ | − |
| 300 | 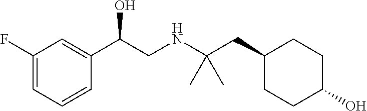 [SMILES: CC(C)(C[C@H]1CC[C@H](O)CC1)NC[C@H](O)c1cccc(F)c1] | (R)-2-{1,1-dimethyl-2- [(1r,4R)-4- hydroxycyclohexyl]ethyl amino}-1-(m- fluorophenyl)-1-ethanol | +++ | − |
| 301 | 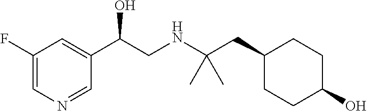 [SMILES: CC(C)(C[C@H]1CC[C@@H](O)CC1)NC[C@H](O)c1cncc(F)c1] | (R)-2-{1,1-dimethyl-2- [(1s,4S)-4- hydroxycyclohexyl]ethyl amino}-1-(5-fluoro-3- pyridyl)-1-ethanol | +++ | − |
| 302 | 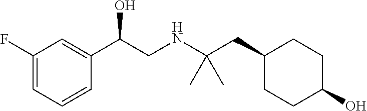 [SMILES: CC(C)(C[C@H]1CC[C@@H](O)CC1)NC[C@H](O)c1cccc(F)c1] | (R)-2-{1,1-dimethyl-2- [(1s,4S)-4- hydroxycyclohexyl]ethyl amino}-1-(m- fluorophenyl)-1-ethanol | ++ | − |
| 303 | 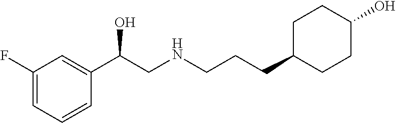 [SMILES: O[C@@H](CNCCC[C@H]1CC[C@H](O)CC1)c1cccc(F)c1] | (R)-1-(m-fluorophenyl)- 2-{3-[(1r,4S)-4- hydroxycyclohexyl] propylamino}-1-ethanol | ++ | − |
| 304 | 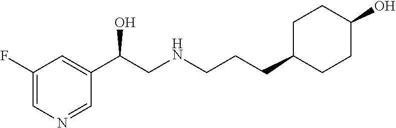 [SMILES: O[C@@H](CNCCC[C@H]1CC[C@@H](O)CC1)c1cncc(F)c1] | (R)-1-(5-fluoro-3- pyridyl)-2-{3-[(1s,4R)- 4- hydroxycyclohexyl] propylamino}-1-ethanol | ++ | − |
| 305 | 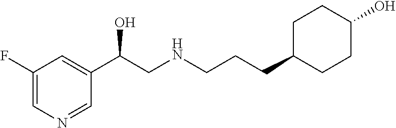 [SMILES: O[C@@H](CNCCC[C@H]1CC[C@H](O)CC1)c1cncc(F)c1] | (R)-1-(5-fluoro-3- pyridyl)-2-{3-[(1r,4S)- 4- hydroxycyclohexyl]prop ylamino}-1-ethanol | + | − |
| 306 | 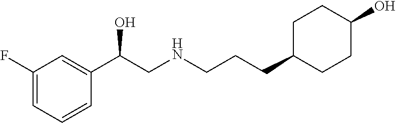 [SMILES: O[C@@H](CNCCC[C@H]1CC[C@@H](O)CC1)c1cccc(F)c1] | (R)-1-(m-fluorophenyl)- 2-{3-[(1s,4R)-4- hydroxycyclohexyl]prop ylamino}-1-ethanol | + | − |
| 307 | 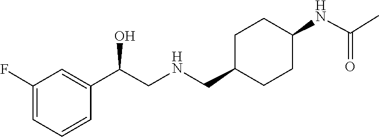 [SMILES: CC(=O)N[C@H]1CC[C@@H](CNC[C@H](O)c2cccc(F)c2)CC1] | N-[(1S,4s)-4-{[(R)-2- (m-fluorophenyl)-2- hydroxyethylamino] methyl}cyclohexyl]] acetamide | ++ | − |
| 308 | 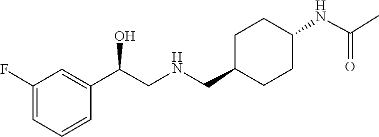 [SMILES: CC(=O)N[C@H]1CC[C@H](CNC[C@H](O)c2cccc(F)c2)CC1] | N-[(1R,4r)-4-{[(R)-2- (m-fluorophenyl)-2- hydroxyethylamino]] methyl}cyclohexyl]] acetamide | ++ | − |
| 309 | 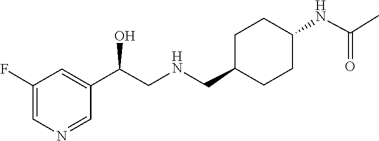 [SMILES: CC(=O)N[C@H]1CC[C@H](CNC[C@H](O)c2cncc(F)c2)CC1] | N-[(1R,4r)-4-{[(R)-2- (5-fluoro-3-pyridyl)-2- hydroxyethylamino] ]methyl}cyclohexyl]] acetamide | ++ | − |
| 310 | 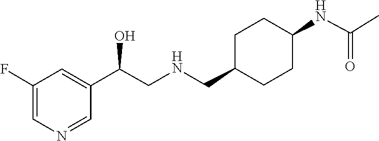 [SMILES: CC(=O)N[C@H]1CC[C@@H](CNC[C@H](O)c2cncc(F)c2)CC1] | N-[(1S,4s)-4-{[(R)-2- (5-fluoro-3-pyridyl)-2- hydroxyethylamino] methyl}cyclohexyl]] acetamide | + | − |
| 311 | 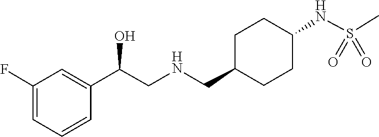 [SMILES: CS(=O)(=O)N[C@H]1CC[C@H](CNC[C@H](O)c2cccc(F)c2)CC1] | (R)-1-(m-fluorophenyl)- 2-({[(1r,4R)-4- (mesylamino)cyclohexyl] ]methyl}amino)-1- ethanol | ++ | − |
| 312 | 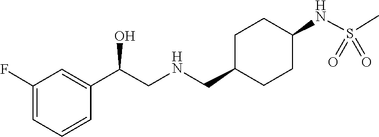 [SMILES: CS(=O)(=O)N[C@H]1CC[C@@H](CNC[C@H](O)c2cccc(F)c2)CC1] | (R)-1-(m-fluorophenyl)- 2-({[(1s,4S)-4- (mesylamino)cyclohexyl] methyl}amino)-1- ethanol | ++ | − |
| 313 | 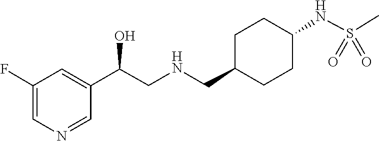 [SMILES: CS(=O)(=O)N[C@H]1CC[C@H](CNC[C@H](O)c2cncc(F)c2)CC1] | (R)-1-(5-fluoro-3- pyridyl)-2-({[(1r,4R)-4- (mesylamino)cyclohexyl] ]methyl}amino)-1- ethanol | + | − |
| 314 | 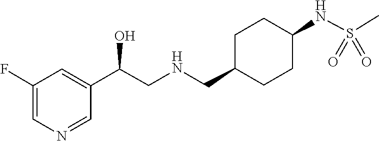 [SMILES: CS(=O)(=O)N[C@H]1CC[C@@H](CNC[C@H](O)c2cncc(F)c2)CC1] | (R)-1-(5-fluoro-3- pyridyl)-2-({[(1s,4S)-4- (mesylamino)cyclohexyl] methyl}amino)-1- ethanol | ++ | − |
| 315 | 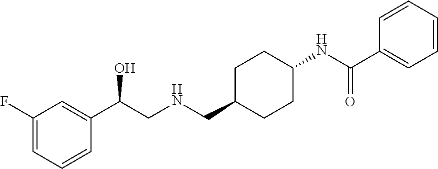 [SMILES: O=C(N[C@H]1CC[C@H](CNC[C@H](O)c2cccc(F)c2)CC1)c1ccccc1] | N-[(1R,4r)-4-{[(R)-2- (m-fluorophenyl)-2- hydroxyethylamino] methyl}cyclohexyl] benzamide | +++ | − |
| 316 | 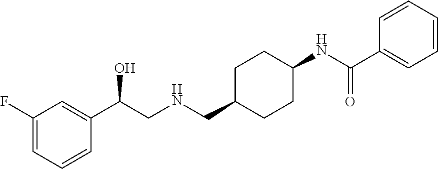 [SMILES: O=C(N[C@H]1CC[C@@H](CNC[C@H](O)c2cccc(F)c2)CC1)c1ccccc1] | N-[(1S,4s)-4-{[(R)-2- (m-fluorophenyl)-2- hydroxyethylamino]] methyl}cyclohexyl] benzamide | ++ | − |
| 317 | 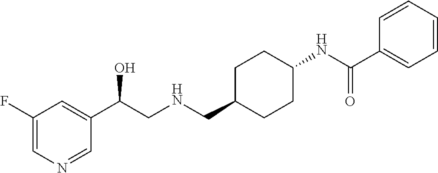 [SMILES: O=C(N[C@H]1CC[C@H](CNC[C@H](O)c2cncc(F)c2)CC1)c1ccccc1] | N-[(1R,4r)-4-{[(R)-2- (5-fluoro-3-pyridyl)-2- hydroxyethylamino]] methyl}cyclohexyl] benzamide | ++ | − |
| 318 | 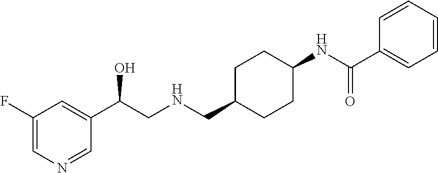 [SMILES: O=C(N[C@H]1CC[C@@H](CNC[C@H](O)c2cncc(F)c2)CC1)c1ccccc1] | N-[(1S,4s)-4-{[(R)-2- (5-fluoro-3-pyridyl)-2- hydroxyethylamino] ]methyl}cyclohexyl] benzamide | ++ | − |
| 319 | 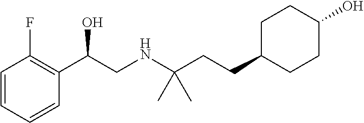 [SMILES: CC(C)(CC[C@H]1CC[C@H](O)CC1)NC[C@H](O)c1ccccc1F] | (R)-2-{1,1-dimethyl-3- [(1r,4S)-4- hydroxycyclohexyl] propylamino}-1-(o- fluorophenyl)-1-ethanol | +++ | − |
| 320 | 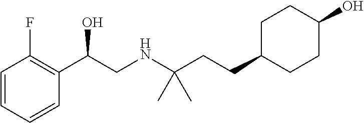 [SMILES: CC(C)(CC[C@H]1CC[C@@H](O)CC1)NC[C@H](O)c1ccccc1F] | (R)-2-{1,1-dimethyl-3- [(1s,4R)-4- hydroxycyclohexyl] propylamino}-1-(o- fluorophenyl)-1-ethanol | +++ | + |
| 321 | 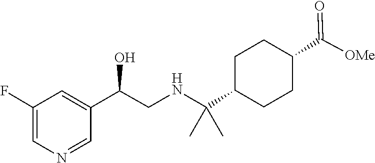 [SMILES: COC(=O)[C@H]1CC[C@@H](C(C)(C)NC[C@H](O)c2cncc(F)c2)CC1] | (1S,4s)-8-[(R)-2-(5- fluoro-3-pyridyl)-2- hydroxyethylamino]-7- methoxy-p-menthan-7- one | ++ | − |
| 322 | 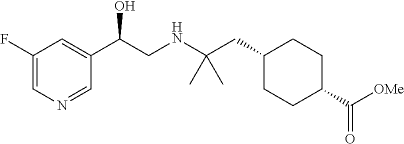 [SMILES: COC(=O)[C@H]1CC[C@@H](CC(C)(C)NC[C@H](O)c2cncc(F)c2)CC1] | methyl (1S,4s)-4-{2- [(R)-2-(5-fluoro-3- pyridyl)-2- hydroxyethylamino]-2- methylpropyl} cyclohexanecarboxylate | +++ | − |
| 323 | 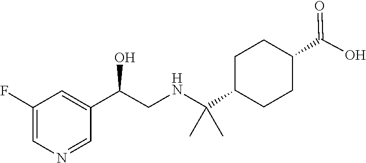 [SMILES: CC(C)(NC[C@H](O)c1cncc(F)c1)[C@H]1CC[C@@H](C(=O)O)CC1] | (1S,4s)-8-[(R)-2-(5- fluoro-3-pyridyl)-2- hydroxyethylamino]-7- hydroxy-p-menthan-7- one | + | ++ |
| 324 | 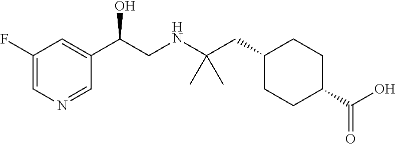 [SMILES: CC(C)(C[C@H]1CC[C@@H](C(=O)O)CC1)NC[C@H](O)c1cncc(F)c1] | (1S,4s)-4-{2-[(R)-2-(5- fluoro-3-pyridyl)-2- hydroxyethylamino]-2- methylpropyl} cyclohexanecarboxylic acid | + | − |
| 325 | 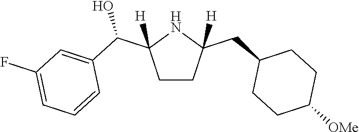 [SMILES: CO[C@H]1CC[C@H](C[C@@H]2CC[C@H]([C@@H](O)c3cccc(F)c3)N2)CC1] | (S)-[(2R,5S)-5- {[(1r,4S)-4- methoxycyclohexyl] methyl}-2-pyrrolidinyl](m- fluorophenyl)methanol | +++ | − |
| 326 | 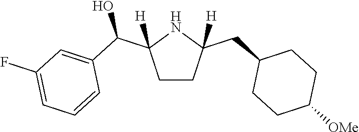 [SMILES: CO[C@H]1CC[C@H](C[C@@H]2CC[C@H]([C@H](O)c3cccc(F)c3)N2)CC1] | (R)-[(2R,5S)-5- {[(1r,4S)-4- methoxycyclohexyl] methyl}-2-pyrrolidinyl] (m-fluorophenyl)methanol | +++ | − |
| 327 | 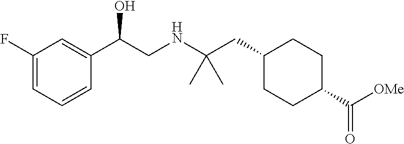 [SMILES: COC(=O)[C@H]1CC[C@@H](CC(C)(C)NC[C@H](O)c2cccc(F)c2)CC1] | methyl (1S,4s)-4-{2- [(R)-2-(m- fluorophenyl)-2- hydroxyethylamino]-2- methylpropyl} cyclohexanecarboxylate | +++ | − |
| 328 | 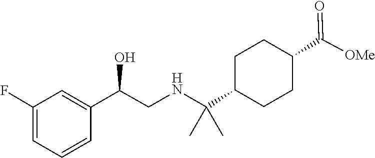 [SMILES: COC(=O)[C@H]1CC[C@@H](C(C)(C)NC[C@H](O)c2cccc(F)c2)CC1] | (1S,4s)-8-[(R)-2-(m- fluorophenyl)-2- hydroxyethylamino]-7- methoxy-p-menthan-7- one | +++ | − |
| 329 | 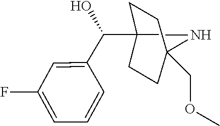 [SMILES: COCC12CCC([C@@H](O)c3cccc(F)c3)(CC1)N2] | (S)-(m-fluorophenyl){4- (methoxymethyl)-7- azabicyclo[[2.2.1]]hept- 1-yl}methanol | + | − |
| 330 | 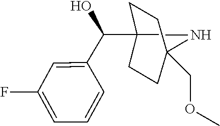 [SMILES: COCC12CCC([C@H](O)c3cccc(F)c3)(CC1)N2] | (R)-(m-fluorophenyl){4- (methoxymethyl)-7- azabicyclo[[2.2.1]]hept- 1-yl}methanol | +++ | − |
| 331 | 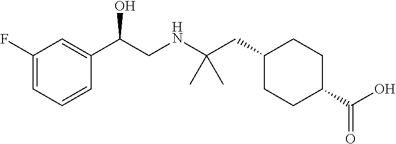 [SMILES: CC(C)(C[C@H]1CC[C@@H](C(=O)O)CC1)NC[C@H](O)c1cccc(F)c1] | (1S,4s)-4-{2-[(R)-2- (m-fluorophenyl)-2- hydroxyethylamino]-2- methylpropyl} cyclohexanecarboxylic acid | + | − |
| 332 | 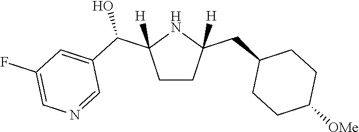 [SMILES: CO[C@H]1CC[C@H](C[C@@H]2CC[C@H]([C@@H](O)c3cncc(F)c3)N2)CC1] | (S)-[(2R,5S)-5- {[(1r,4S)-4- methoxycyclohexyl] methyl}-2-pyrrolidinyl]] (5-fluoro-3- pyridyl)methanol | + | − |
| 333 | 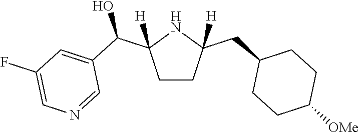 [SMILES: CO[C@H]1CC[C@H](C[C@@H]2CC[C@H]([C@H](O)c3cncc(F)c3)N2)CC1] | (R)-[(2R,5S)-5- {[(1r,4S)-4- methoxycyclohexyl] methyl}-2-pyrrolidinyl] ](5-fluoro-3- pyridyl)methanol | + | − |
| 334 | 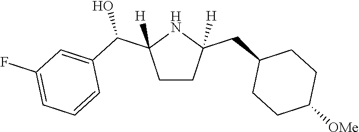 [SMILES: CO[C@H]1CC[C@H](C[C@H]2CC[C@H]([C@@H](O)c3cccc(F)c3)N2)CC1] | (S)-[(2R,5R)-5- {[(1r,4R)-4- methoxycyclohexyl] methyl}-2-pyrrolidinyl] (m- fluorophenyl)methanol | + | − |
| 335 | 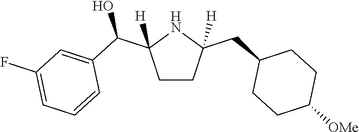 [SMILES: CO[C@H]1CC[C@H](C[C@H]2CC[C@H]([C@H](O)c3cccc(F)c3)N2)CC1] | (R)-[(2R,5R)-5- {[(1r,4R)-4- methoxycyclohexyl] methyl}-2-pyrrolidinyl]] (m-fluorophenyl)methanol | +++ | − |
| 336 | 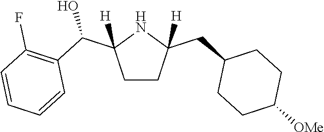 [SMILES: CO[C@H]1CC[C@H](C[C@@H]2CC[C@H]([C@@H](O)c3ccccc3F)N2)CC1] | (S)-[(2R,5S)-5- {[(1r,4S)-4- methoxycyclohexyl] methyl}-2-pyrrolidinyl] (o-fluorophenyl)methanol | + | − |
| 337 | 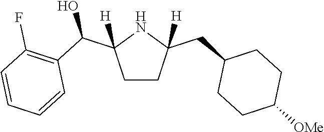 [SMILES: CO[C@H]1CC[C@H](C[C@@H]2CC[C@H]([C@H](O)c3ccccc3F)N2)CC1] | (R)-[(2R,5S)-5- {[(1r,4S)-4- methoxycyclohexyl] methyl}-2-pyrrolidinyl] (o-fluorophenyl)methanol | +++ | − |
| 338 | 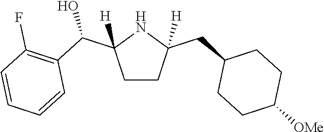 [SMILES: CO[C@H]1CC[C@H](C[C@H]2CC[C@H]([C@@H](O)c3ccccc3F)N2)CC1] | (S)-[(2R,5R)-5- {[(1r,4R)-4- methoxycyclohexyl] methyl}-2- pyrrolidinyl](o- fluorophenyl)methanol | ++ | − |
| 339 | 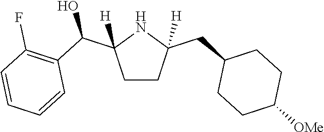 [SMILES: CO[C@H]1CC[C@H](C[C@H]2CC[C@H]([C@H](O)c3ccccc3F)N2)CC1] | (R)-[(2R,5R)-5- {[(1r,4R)-4- methoxycyclohexyl] methyl}-2- pyrrolidinyl](o- fluorophenyl)methanol | +++ | − |
| 340 | 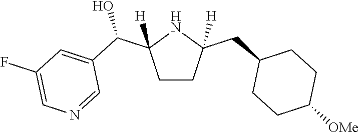 [SMILES: CO[C@H]1CC[C@H](C[C@H]2CC[C@H]([C@@H](O)c3cncc(F)c3)N2)CC1] | (S)-[(2R,5R)-5- {[(1r,4R)-4- methoxycyclohexyl] methyl}-2- pyrrolidinyl](5- fluoro-3- pyridyl)methanol | + | − |
| 341 | 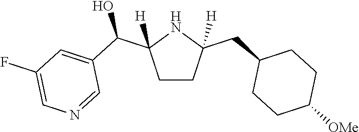 [SMILES: CO[C@H]1CC[C@H](C[C@H]2CC[C@H]([C@H](O)c3cncc(F)c3)N2)CC1] | (R)-[(2R,5R)-5- {[(1r,4R)-4- methoxycyclohexyl] methyl}-2- pyrrolidinyl]](5- fluoro-3- pyridyl)methanol | +++ | − |
| 342 | 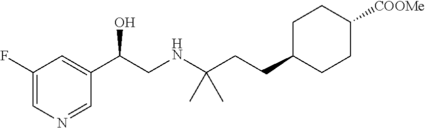 [SMILES: COC(=O)[C@H]1CC[C@H](CCC(C)(C)NC[C@H](O)c2cncc(F)c2)CC1] | methyl (1R,4r)-4-{3- [(R)-2-(5-fluoro-3- pyridyl)-2- hydroxyethylamino]-3- methylbutyl} cyclohexanecarboxylate | +++ | − |
| 343 | 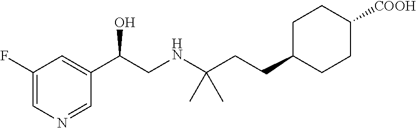 [SMILES: CC(C)(CC[C@H]1CC[C@H](C(=O)O)CC1)NC[C@H](O)c1cncc(F)c1] | (1R,4r)-4-{3-[(R)-2-(5- fluoro-3-pyridyl)-2- hydroxyethylamino]-3- methylbutyl} cyclohexanecarboxylic acid | + | − |
| 344 | 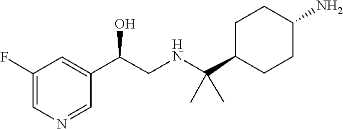 [SMILES: CC(C)(NC[C@H](O)c1cncc(F)c1)[C@H]1CC[C@H](N)CC1] | (R)-1-(5-fluoro-3- pyridyl)-2-{1-methyl-1- [(1r,4R)-4- aminocyclohexyl]] ethylamino}-1-ethanol | − | − |
| 345 | 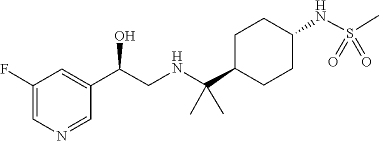 [SMILES: CC(C)(NC[C@H](O)c1cncc(F)c1)[C@H]1CC[C@H](NS(C)(=O)=O)CC1] | (R)-1-(5-fluoro-3- pyridyl)-2-{1-methyl-1- [(1r,4R)-4- (mesylamino)cyclohexyl ]ethylamino}-1-ethanol | + | − |
| 346 | 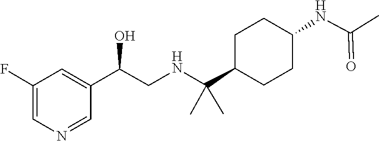 [SMILES: CC(=O)N[C@H]1CC[C@H](C(C)(C)NC[C@H](O)c2cncc(F)c2)CC1] | N-[(1R,4r)-4-{1-[(R)-2- (5-fluoro-3-pyridyl)-2- hydroxyethylamino]-1- methylethyl}cyclohexyl] acetamide | +++ | − |
| 347 | 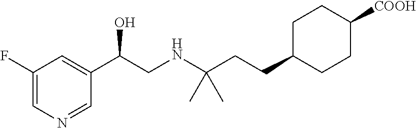 [SMILES: CC(C)(CC[C@H]1CC[C@@H](C(=O)O)CC1)NC[C@H](O)c1cncc(F)c1] | (1S,4s)-4-{3-[(R)-2-(5- fluoro-3-pyridyl)-2- hydroxyethylamino]-3- methylbutyl} cyclohexanecarboxylic acid | +++ | − |
| 348 | 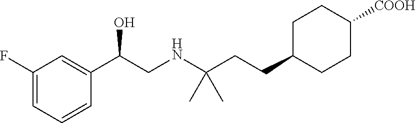 [SMILES: CC(C)(CC[C@H]1CC[C@H](C(=O)O)CC1)NC[C@H](O)c1cccc(F)c1] | (1R,4r)-4-{3-[(R)-2- (m-fluorophenyl)-2- hydroxyethylamino]-3- methylbutyl} cyclohexanecarboxylic acid | − | − |
| 349 | 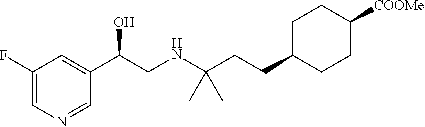 [SMILES: COC(=O)[C@H]1CC[C@@H](CCC(C)(C)NC[C@H](O)c2cncc(F)c2)CC1] | methyl (1S,4s)-4-{3- [(R)-2-(5-fluoro-3- pyridyl)-2- hydroxyethylamino]-3- methylbutyl} cyclohexanecarboxylate | +++ | + |
| 350 | 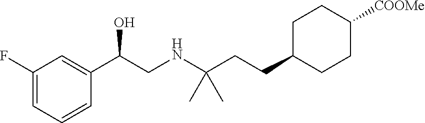 [SMILES: COC(=O)[C@H]1CC[C@H](CCC(C)(C)NC[C@H](O)c2cccc(F)c2)CC1] | methyl (1R,4r)-4-{3- [(R)-2-(m- fluorophenyl)-2- hydroxyethylamino]-3- methylbutyl} cyclohexanecarboxylate | +++ | − |
| 351 | 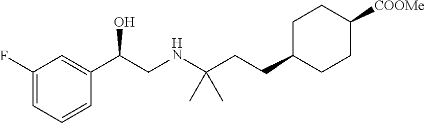 [SMILES: COC(=O)[C@H]1CC[C@@H](CCC(C)(C)NC[C@H](O)c2cccc(F)c2)CC1] | methyl (1S,4s)-4-{3- [(R)-2-(m- fluorophenyl)-2- hydroxyethylamino]-3- methylbutyl} cyclohexanecarboxylate | +++ | + |
| 352 | 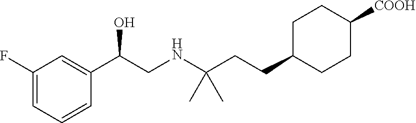 [SMILES: CC(C)(CC[C@H]1CC[C@@H](C(=O)O)CC1)NC[C@H](O)c1cccc(F)c1] | (1S,4s)-4-{3-[(R)-2- (m-fluorophenyl)-2- hydroxyethylamino]-3- methylbutyl} cyclohexanecarboxylic acid | + | − |
| 353 | 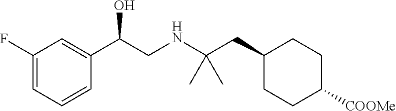 [SMILES: COC(=O)[C@H]1CC[C@H](CC(C)(C)NC[C@H](O)c2cccc(F)c2)CC1] | methyl (1R,4r)-4-{2- [(R)-2-(m- fluorophenyl)-2- hydroxyethylamino]-2- methylpropyl} cyclohexanecarboxylate | +++ | − |
| 354 | 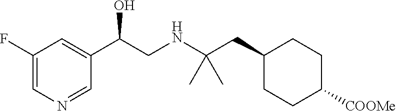 [SMILES: COC(=O)[C@H]1CC[C@H](CC(C)(C)NC[C@H](O)c2cncc(F)c2)CC1] | methyl (1R,4r)-4-{2- [(R)-2-(5-fluoro-3- pyridyl)-2- hydroxyethylamino]-2- methylpropyl} cyclohexanecarboxylate | ++ | − |
| 355 | 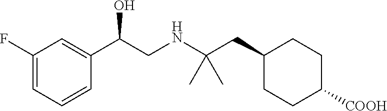 [SMILES: CC(C)(C[C@H]1CC[C@H](C(=O)O)CC1)NC[C@H](O)c1cccc(F)c1] | (1R,4r)-4-{2-[(R)-2- (m-fluorophenyl)-2- hydroxyethylamino]-2- methylpropyl} cyclohexanecarboxylic acid | ++ | − |
| 356 | 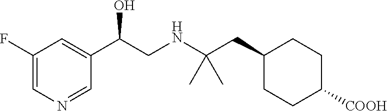 [SMILES: CC(C)(C[C@H]1CC[C@H](C(=O)O)CC1)NC[C@H](O)c1cncc(F)c1] | (1R,4r)-4-{2-[(R)-2-(5- fluoro-3-pyridyl)-2- hydroxyethylamino]-2- methylpropyl} cyclohexanecarboxylic acid | − | − |
| 357 | 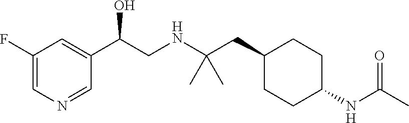 [SMILES: CC(=O)N[C@H]1CC[C@H](CC(C)(C)NC[C@H](O)c2cncc(F)c2)CC1] | N-[(1R,4r)-4-{2-[(R)-2- (5-fluoro-3-pyridyl)-2- hydroxyethylamino]-2- methylpropyl}cyclohexyl] ]acetamide | +++ | − |
| 358 | 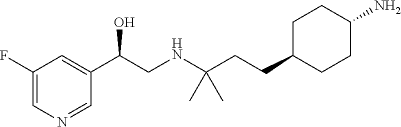 [SMILES: CC(C)(CC[C@H]1CC[C@H](N)CC1)NC[C@H](O)c1cncc(F)c1] | (R)-2-{1,1-dimethyl-3- [(1r,4R)-4- aminocyclohexyl]]propyl amino}-1-(5-fluoro-3- pyridyl)-1-ethanol | + | − |
| 359 | 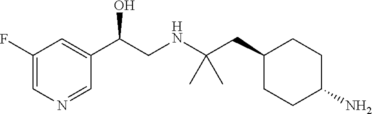 [SMILES: CC(C)(C[C@H]1CC[C@H](N)CC1)NC[C@H](O)c1cncc(F)c1] | (R)-2-{1,1-dimethyl-2- [(1r,4R)-4- aminocyclohexyl] ethylamino}-1-(5- fluoro-3- pyridyl)-1-ethanol | +++ | − |
| 360 | 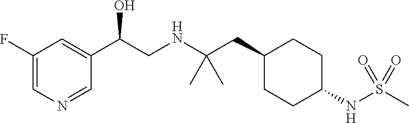 [SMILES: CC(C)(C[C@H]1CC[C@H](NS(C)(=O)=O)CC1)NC[C@H](O)c1cncc(F)c1] | (R)-2-{1,1-dimethyl-2- [(1r,4R)-4- (mesylamino)cyclohexyl] ethylamino}-1-(5- fluoro-3-pyridyl)-1- ethanol | +++ | − |
| 361 | 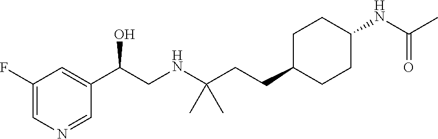 [SMILES: CC(=O)N[C@H]1CC[C@H](CCC(C)(C)NC[C@H](O)c2cncc(F)c2)CC1] | N-[(1R,4r)-4-{3-[(R)-2- (5-fluoro-3-pyridyl)-2- hydroxyethylamino]-3- methylbutyl}cyclohexyl] acetamide | +++ | − |
| 362 | 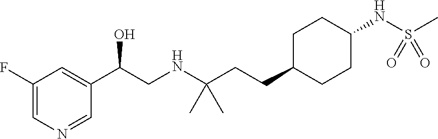 [SMILES: CC(C)(CC[C@H]1CC[C@H](NS(C)(=O)=O)CC1)NC[C@H](O)c1cncc(F)c1] | (R)-2-{1,1-dimethyl-3- [(1r,4R)-4- (mesylamino)cyclohexyl] propylamino}-1-(5- fluoro-3-pyridyl)-1- ethanol | ++ | − |
| 363 | 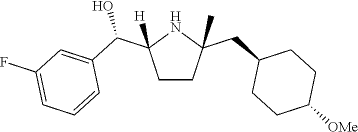 [SMILES: CO[C@H]1CC[C@H](C[C@]2(C)CC[C@H]([C@@H](O)c3cccc(F)c3)N2)CC1] | (S)-[(2R,5S)-5-methyl- 5-{[(1r,4S)-4- methoxycyclohexyl] methyl}-2-pyrrolidinyl](m- fluorophenyl)methanol | +++ | − |
| 364 | 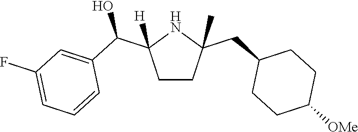 [SMILES: CO[C@H]1CC[C@H](C[C@]2(C)CC[C@H]([C@H](O)c3cccc(F)c3)N2)CC1] | (R)-[(2R,5S)-5-methyl- 5-{[(1r,4S)-4- methoxycyclohexyl] methyl}-2-pyrrolidinyl] (m-fluorophenyl)methanol | +++ | ++ |
| 365 | 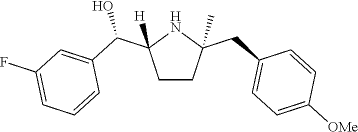 | (S)-{(2R,5R)-5-[(p- methoxyphenyl)methyl]- 5-methyl-2- pyrrolidinyl}(m- fluorophenyl)methanol | +++ | − |
| 366 | 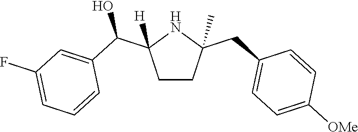 | (R)-{(2R,5R)-5-[(p- methoxyphenyl)methyl]- 5-methyl-2- pyrrolidinyl}(m- fluorophenyl)methanol | +++ | + |
| 367 | 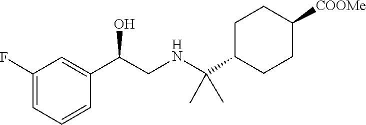 [SMILES: COC(=O)[C@H]1CC[C@H](C(C)(C)NC[C@H](O)c2cccc(F)c2)CC1] | (1R,4r)-8-[(R)-2-(m- fluorophenyl)-2- hydroxyethylamino]-7- methoxy-p-menthan-7- one | +++ | + |
| 368 | 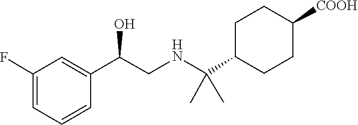 [SMILES: CC(C)(NC[C@H](O)c1cccc(F)c1)[C@H]1CC[C@H](C(=O)O)CC1] | (1R,4r)-8-[(R)-2-(m- fluorophenyl)-2- hydroxyethylamino]-7- hydroxy-p-menthan-7- one | + | − |
| 369 | 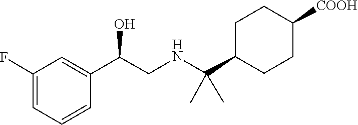 [SMILES: CC(C)(NC[C@H](O)c1cccc(F)c1)[C@H]1CC[C@@H](C(=O)O)CC1] | (1S,4s)-8-[(R)-2-(m- fluorophenyl)-2- hydroxyethylamino]]-7- hydroxy-p-menthan-7- one | ++ | − |
| 370 | 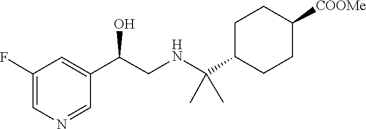 [SMILES: COC(=O)[C@H]1CC[C@H](C(C)(C)NC[C@H](O)c2cncc(F)c2)CC1] | (1R,4r)-8-[(R)-2-(5- fluoro-3-pyridyl)-2- hydroxyethylamino]-7- methoxy-p-menthan-7- one | +++ | − |
| 371 | 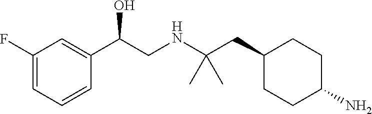 [SMILES: CC(C)(C[C@H]1CC[C@H](N)CC1)NC[C@H](O)c1cccc(F)c1] | (R)-2-{1,1-dimethyl-2- [(1r,4R)-4- aminocyclohexyl]] ethylamino}-1-(m- fluorophenyl)-1-ethanol | +++ | − |
| 372 | 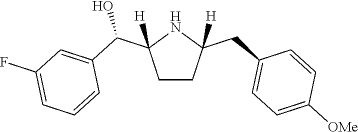 | (S)-{(2R,5S)-5-[(p- methoxyphenyl)methyl]- 2-pyrrolidinyl}(m- fluorophenyl)methanol | +++ | − |
| 373 | 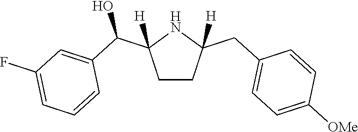 [SMILES: COc1ccc(C[C@@H]2CC[C@H]([C@H](O)c3cccc(F)c3)N2)cc1] | (R)-{(2R,5S)-5-[(p- methoxyphenyl)methyl]- 2-pyrrolidinyl}(m- fluorophenyl)methanol | +++ | + |
| 374 | 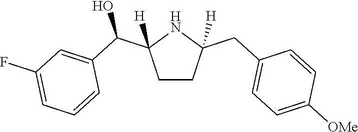 [SMILES: COc1ccc(C[C@H]2CC[C@H]([C@H](O)c3cccc(F)c3)N2)cc1] | (R)-{(2R,5R)-5-[(p- methoxyphenyl)methyl]- 2-pyrrolidinyl}(m- fluorophenyl)methanol | +++ | − |
| 375 | 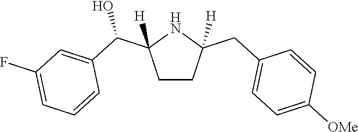 [SMILES: COc1ccc(C[C@H]2CC[C@H]([C@@H](O)c3cccc(F)c3)N2)cc1] | (S)-{(2R,5R)-5-[(p- methoxyphenyl)methyl]- 2-pyrrolidinyl}(m- fluorophenyl)methanol | +++ | − |
| 376 | 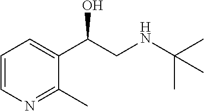 | (R)-2-(tert-butylamino)- 1-(2-methyl-3-pyridyl)- 1-ethanol | +++ | + |
| 377 | 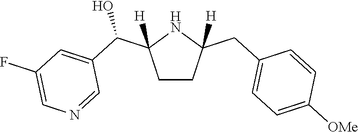 [SMILES: COc1ccc(C[C@@H]2CC[C@H]([C@@H](O)c3cncc(F)c3)N2)cc1] | (S)-{(2R,5S)-5-[(p- methoxyphenyl)methyl]- 2-pyrrolidinyl}(5- fluoro-3- pyridyl)methanol | +++ | − |
| 378 | 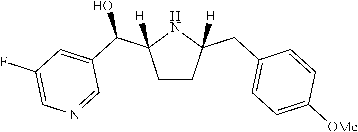 [SMILES: COc1ccc(C[C@@H]2CC[C@H]([C@H](O)c3cncc(F)c3)N2)cc1] | (R)-{(2R,5S)-5-[(p- methoxyphenyl)methyl]- 2-pyrrolidinyl}(5- fluoro-3- pyridyl)methanol | +++ | − |
| 379 | 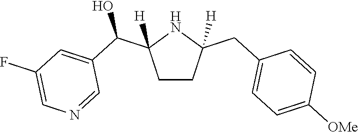 [SMILES: COc1ccc(C[C@H]2CC[C@H]([C@H](O)c3cncc(F)c3)N2)cc1] | (R)-{(2R,5R)-5-[(p- methoxyphenyl)methyl]- 2-pyrrolidinyl}(5- fluoro-3- pyridyl)methanol | +++ | − |
| 380 | 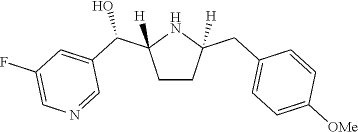 [SMILES: COc1ccc(C[C@H]2CC[C@H]([C@@H](O)c3cncc(F)c3)N2)cc1] | (S)-{(2R,5R)-5-[(p- methoxyphenyl)methyl]- 2-pyrrolidinyl}(5- fluoro-3- pyridyl)methanol | +++ | − |
| 381 | 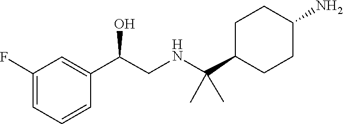 [SMILES: CC(C)(NC[C@H](O)c1cccc(F)c1)[C@H]1CC[C@H](N)CC1] | (R)-1-(m-fluorophenyl)- 2-{1-methyl-1- [(1r,4R)-4- aminocyclohexyl]] ethylamino}-1-ethanol | ++ | − |
| 382 | 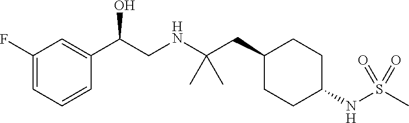 [SMILES: CC(C)(C[C@H]1CC[C@H](NS(C)(=O)=O)CC1)NC[C@H](O)c1cccc(F)c1] | (R)-2-{1,1-dimethyl-2- [(1r,4R)-4- (mesylamino)cyclohexyl] ]ethylamino}-1-(m- fluorophenyl)-1-ethanol | +++ | + |
| 383 | 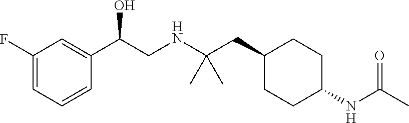 [SMILES: CC(=O)N[C@H]1CC[C@H](CC(C)(C)NC[C@H](O)c2cccc(F)c2)CC1] | N-[(1R,4r)-4-{2-[(R)-2- (m-fluorophenyl)-2- hydroxyethylamino]-2- methylpropyl}cyclohexy l]]acetamide | +++ | + |
| 384 | 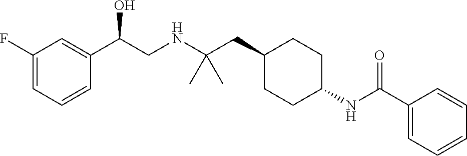 [SMILES: CC(C)(C[C@H]1CC[C@H](NC(=O)c2ccccc2)CC1)NC[C@H](O)c1cccc(F)c1] | N-[(1R,4r)-4-{2-[(R)-2- (m-fluorophenyl)-2- hydroxyethylamino]-2- methylpropyl}cyclohexyl] ]benzamide | +++ | +++ |
| 385 | 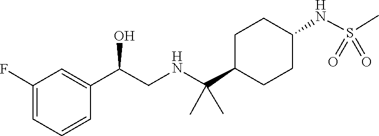 [SMILES: CC(C)(NC[C@H](O)c1cccc(F)c1)[C@H]1CC[C@H](NS(C)(=O)=O)CC1] | (R)-1-(m-fluorophenyl)- 2-{1-methyl-1- [(1r,4R)-4- (mesylamino)cyclohexyl] ethylamino}-1-ethanol | ++ | − |
| 386 | 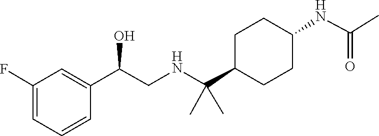 [SMILES: CC(=O)N[C@H]1CC[C@H](C(C)(C)NC[C@H](O)c2cccc(F)c2)CC1] | N-[(1R,4r)-4-{1-[(R)-2- (m-fluorophenyl)-2- hydroxyethylamino]-1- methylethyl}cyclohexyl] acetamide | +++ | − |
| 387 | 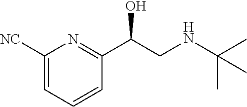 [SMILES: CC(C)(C)NC[C@H](O)c1cccc(C#N)n1] | 6-[(S)-2-(tert- butylamino)-1- hydroxyethyl]-2- pyridinecarbonitrile | +++ | + |
| 388 | 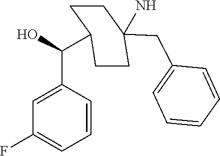 [SMILES: NC1(Cc2ccccc2)CCC([C@H](O)c2cccc(F)c2)CC1] | (R)-(4-benzyl-7- azabicyclo[2.2.1]]hept- 1-yl)(m- fluorophenyl)methanol | +++ | + |
| 389 | 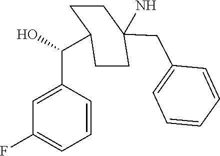 [SMILES: NC1(Cc2ccccc2)CCC([C@@H](O)c2cccc(F)c2)CC1] | (S)-(4-benzyl-7- azabicyclo[[2.2.1]]hept- 1-yl)(m- fluorophenyl)methanol | ++ | − |
| 390 | 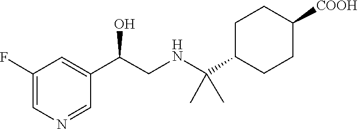 [SMILES: CC(C)(NC[C@H](O)c1cncc(F)c1)[C@H]1CC[C@H](C(=O)O)CC1] | (1R,4r)-8-[(R)-2-(5- fluoro-3-pyridyl)-2- hydroxyethylamino]-7- hydroxy-p-menthan-7- one | ++ | − |
| 391 | 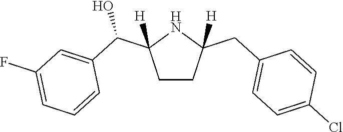 [SMILES: O[C@@H](c1cccc(F)c1)[C@H]1CC[C@@H](Cc2ccc(Cl)cc2)N1] | (S)-{(2R,5S)-5-[(p- chlorophenyl)methyl]-2- pyrrolidinyl}(m- fluorophenyl)methanol | ++ | − |
| 392 | 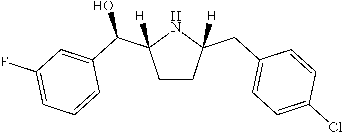 [SMILES: O[C@H](c1cccc(F)c1)[C@H]1CC[C@@H](Cc2ccc(Cl)cc2)N1] | (R)-{(2R,5S)-5-[(p- chlorophenyl)methyl]-2- pyrrolidinyl}(m- fluorophenyl)methanol | +++ | − |
| 393 | 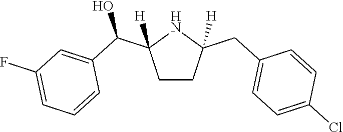 [SMILES: O[C@H](c1cccc(F)c1)[C@H]1CC[C@H](Cc2ccc(Cl)cc2)N1] | (R)-{(2R,5R)-5-[(p- chlorophenyl)methyl]-2- pyrrolidinyl}(m- fluorophenyl)methanol | +++ | − |
| 394 | 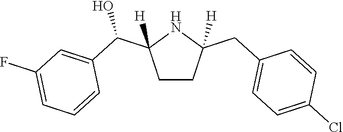 [SMILES: O[C@@H](c1cccc(F)c1)[C@H]1CC[C@H](Cc2ccc(Cl)cc2)N1] | (S)-{(2R,5R)-5-[(p- chlorophenyl)methyl]-2- pyrrolidinyl}(m- fluorophenyl)methanol | ++ | − |
| 395 | 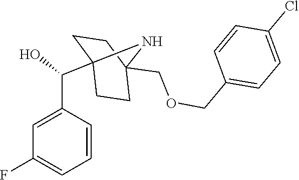 [SMILES: O[C@@H](c1cccc(F)c1)C12CCC(COCc3ccc(Cl)cc3)(CC1)N2] | (S)-(4-{[(p- chlorophenyl)methoxy] methyl}-7- azabicyclo[[2.2.1]]hept- 1-yl)(m- fluorophenyl)methanol | ++ | − |
| 396 | 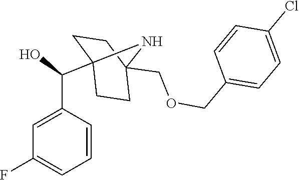 [SMILES: O[C@H](c1cccc(F)c1)C12CCC(COCc3ccc(Cl)cc3)(CC1)N2] | (R)-(4-{[(p- chlorophenyl)methoxy] methyl}-7- azabicyclo[[2.2.1]]hept- 1-yl)(m- fluorophenyl)methanol | +++ | − |
| 397 | 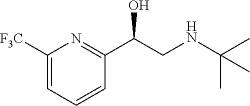 [SMILES: CC(C)(C)NC[C@H](O)c1cccc(C(F)(F)F)n1] | (S)-2-(tert-butylamino)- 1-[6-(trifluoromethyl)- 2-pyridyl]-1-ethanol | +++ | − |
| 398 | 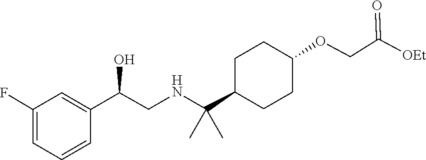 [SMILES: CCOC(=O)CO[C@H]1CC[C@H](C(C)(C)NC[C@H](O)c2cccc(F)c2)CC1] | ethyl[[(1R,4r)-4-{1- [(R)-2-(m- fluorophenyl)-2- hydroxyethylamino]-1- methylethyl}cyclohexyl oxy]]acetate | +++ | − |
| 399 | 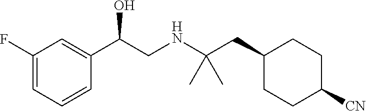 [SMILES: CC(C)(C[C@H]1CC[C@@H](C#N)CC1)NC[C@H](O)c1cccc(F)c1] | (1S,4s)-4-{2-[(R)-2- (m-fluorophenyl)-2- hydroxyethylamino]-2- methylpropyl} cyclohexanecarbonitrile | +++ | + |
| 400 | 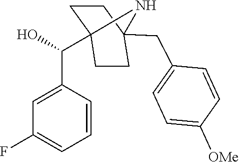 [SMILES: COc1ccc(CC23CCC([C@@H](O)c4cccc(F)c4)(CC2)N3)cc1] | (S)-(m-fluorophenyl){4- [(p- methoxyphenyl)methyl]- 7- azabicyclo[[2.2.1]]hept- 1-yl}methanol | ++ | − |
| 401 | 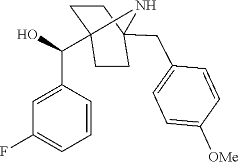 [SMILES: COc1ccc(CC23CCC([C@H](O)c4cccc(F)c4)(CC2)N3)cc1] | (R)-(m-fluorophenyl){4- [(p- methoxyphenyl)methyl]- 7- azabicyclo[[2.2.1]]hept- 1-yl}methanol | +++ | − |
| 402 | 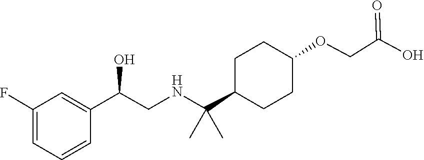 [SMILES: CC(C)(NC[C@H](O)c1cccc(F)c1)[C@H]1CC[C@H](OCC(=O)O)CC1] | [(1R,4r)-4-{1-[(R)-2- (m-fluorophenyl)-2- hydroxyethylamino]-1- methylethyl}cyclohexyl oxy]]acetic acid | ++ | − |
| 403 | 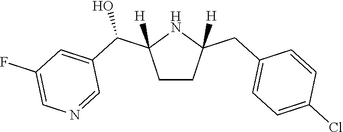 [SMILES: O[C@@H](c1cncc(F)c1)[C@H]1CC[C@@H](Cc2ccc(Cl)cc2)N1] | (S)-{(2R,5S)-5-[(p- chlorophenyl)methyl]-2- pyrrolidinyl}(5-fluoro-3- pyridyl)methanol | ++ | − |
| 404 | 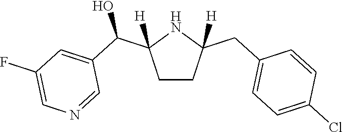 [SMILES: O[C@H](c1cncc(F)c1)[C@H]1CC[C@@H](Cc2ccc(Cl)cc2)N1] | (R)-{(2R,5S)-5-[(p- chlorophenyl)methyl]-2- pyrrolidinyl}(5-fluoro-3- pyridyl)methanol | +++ | − |
| 405 | 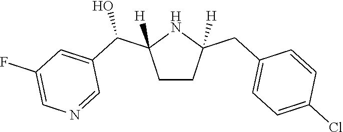 [SMILES: O[C@@H](c1cncc(F)c1)[C@H]1CC[C@H](Cc2ccc(Cl)cc2)N1] | (S)-{(2R,5R)-5-[(p- chlorophenyl)methyl]-2- pyrrolidinyl}(5-fluoro-3- pyridyl)methanol | +++ | − |
| 406 | 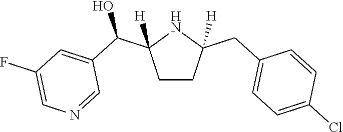 [SMILES: O[C@H](c1cncc(F)c1)[C@H]1CC[C@H](Cc2ccc(Cl)cc2)N1] | (R)-{(2R,5R)-5-[(p- chlorophenyl)methyl]-2- pyrrolidinyl}(5-fluoro-3- pyridyl)methanol | +++ | − |
| 407 | 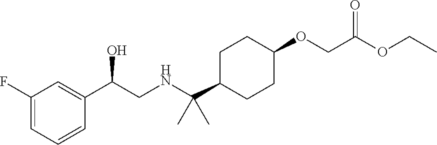 [SMILES: CCOC(=O)CO[C@H]1CC[C@@H](C(C)(C)NC[C@H](O)c2cccc(F)c2)CC1] | ethyl[[(1S,4s)-4-{1- [(R)-2-(m- fluorophenyl)-2- hydroxyethylamino]-1- methylethyl} cyclohexyloxy]]acetate | ++ | − |
| 408 | 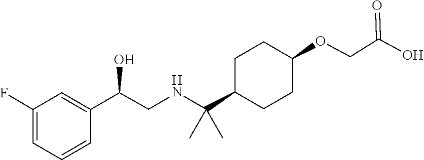 [SMILES: CC(C)(NC[C@H](O)c1cccc(F)c1)[C@H]1CC[C@@H](OCC(=O)O)CC1] | [(1S,4s)-4-{1-[(R)-2- (m-fluorophenyl)-2- hydroxyethylamino]-1- methylethyl} cyclohexyloxy]]acetic acid | ++ | − |
| 409 | 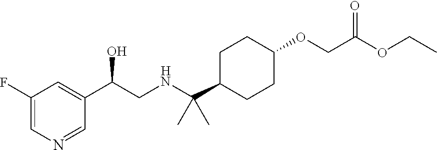 [SMILES: CCOC(=O)CO[C@H]1CC[C@H](C(C)(C)NC[C@H](O)c2cncc(F)c2)CC1] | ethyl[[(1R,4r)-4-{1- [(R)-2-(5-fluoro-3- pyridyl)-2- hydroxyethylamino]-1- methylethyl} cyclohexyloxy]]acetate | +++ | − |
| 410 | 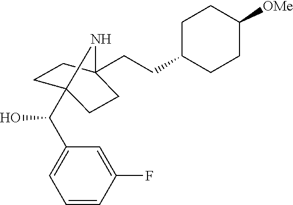 [SMILES: CO[C@H]1CC[C@H](CCC23CCC([C@@H](O)c4cccc(F)c4)(CC2)N3)CC1] | (S)-(m-fluorophenyl)(4- {2-[(1r,4R)-4- methoxycyclohexylethyl}- 7- azabicyclo[[2.2.1]]hept- 1-yl)methanol | ++ | − |
| 411 | 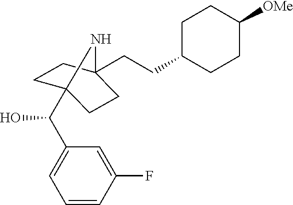 [SMILES: CO[C@H]1CC[C@H](CCC23CCC([C@@H](O)c4cccc(F)c4)(CC2)N3)CC1] | (R)-(m-fluorophenyl)(4- {2-[(1r,4S)-4- methoxycyclohexyl]]ethyl}- 7- azabicyclo[[2.2.1]]hept- 1-yl)methanol | +++ | ++ |
| 412 | 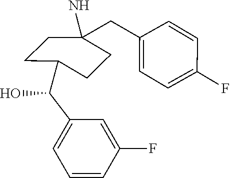 [SMILES: NC1(Cc2ccc(F)cc2)CCC([C@@H](O)c2cccc(F)c2)CC1] | (S)-(m-fluorophenyl){4- [(p- fluorophenyl)methyl]-7- azabicyclo[[2.2.1]]hept- 1-yl}methanol | ++ | − |
| 413 | 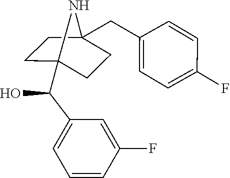 [SMILES: O[C@H](c1cccc(F)c1)C12CCC(Cc3ccc(F)cc3)(CC1)N2] | (R)-(m-fluorophenyl){4- [(p- fluorophenyl)methyl]-7- azabicyclo[[2.2.1]]hept- 1-yl}methanol | +++ | − |
| 414 | 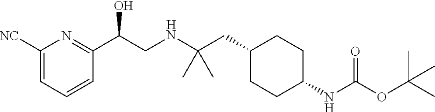 [SMILES: CC(C)(C[C@H]1CC[C@@H](NC(=O)OC(C)(C)C)CC1)NC[C@H](O)c1cccc(C#N)n1] | (1R,4s)-4-{2-[(S)-2-(6- cyano-2-pyridyl)-2- hydroxyethylamino]-2- methylpropyl}cyclohexly 2-methyl-2- propanecarbamate | ++ | − |
| 415 | 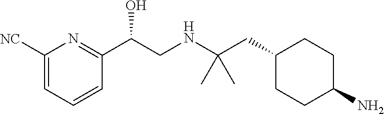 [SMILES: CC(C)(C[C@H]1CC[C@H](N)CC1)NC[C@@H](O)c1cccc(C#N)n1] | 6-[(R)-2-{1,1-dimethyl- 2-[(1r,4R)-4- aminocyclohexyl]] ethylamino}-1- hydroxyethyl]- 2-pyridinecarbonitrile | ++ | − |
| 416 | 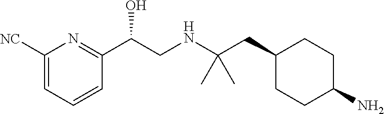 [SMILES: CC(C)(C[C@H]1CC[C@@H](N)CC1)NC[C@@H](O)c1cccc(C#N)n1] | 6-[(R)-2-{1,1-dimethyl- 2-[(1s,4S)-4- aminocyclohexyl]] ethylamino}-1- hydroxyethyl]- 2-pyridinecarbonitrile | ++ | − |
| 417 | 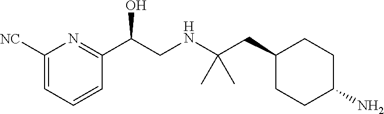 [SMILES: CC(C)(C[C@H]1CC[C@H](N)CC1)NC[C@H](O)c1cccc(C#N)n1] | 6-[(S)-2-{1, 1-dimethyl- 2-[(1r,4S)-4- aminocyclohexyl]] ethylamino}-1- hydroxyethyl]- 2-pyridinecarbonitrile | +++ | + |
| 418 | 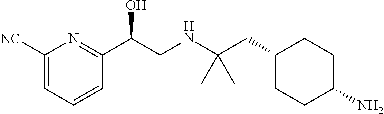 [SMILES: CC(C)(C[C@H]1CC[C@@H](N)CC1)NC[C@H](O)c1cccc(C#N)n1] | 6-[(S)-2-{1, 1-dimethyl- 2-[(1s,4R)-4- aminocyclohexyl] ethylamino}-1- hydroxyethyl]- 2-pyridinecarbonitrile | ++ | − |
| 419 | 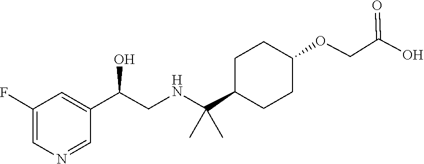 [SMILES: CC(C)(NC[C@H](O)c1cncc(F)c1)[C@H]1CC[C@H](OCC(=O)O)CC1] | [(1R,4r)-4-{1-[(R)-2- (5-fluoro-3-pyridyl)-2- hydroxyethylamino]-1- methylethyl} cyclohexyloxy]]acetic acid | ++ | − |
| 420 | 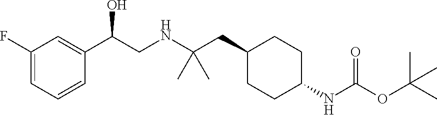 [SMILES: CC(C)(C[C@H]1CC[C@H](NC(=O)OC(C)(C)C)CC1)NC[C@H](O)c1cccc(F)c1] | (1R,4r)-4-{2-[(R)-2- (m-fluorophenyl)-2- hydroxyethylamino]-2- methylpropyl}cyclohexy- 2-methyl-2- propanecarbamate | ++ | + |
| 421 | 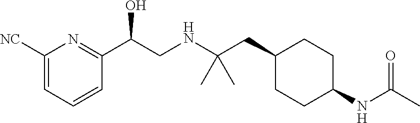 [SMILES: CC(=O)N[C@H]1CC[C@@H](CC(C)(C)NC[C@H](O)c2cccc(C#N)n2)CC1] | N-[(1R,4s)-4-{2-[(S)-2- (6-cyano-2-pyridyl)-2- hydroxyethylamino]-2- methylpropyl}cyclohexyl] ]acetamide | +++ | − |
| 422 | 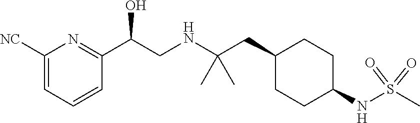 [SMILES: CC(C)(C[C@H]1CC[C@@H](NS(C)(=O)=O)CC1)NC[C@H](O)c1cccc(C#N)n1] | 6-[(S)-2-{1, 1-dimethyl- 2-[(1s,4R)-4- (mesylamino)cyclohexyl] ethylamino}-1- hydroxyethyl]-2- pyridinecarbonitrile | ++ | − |
| 423 | 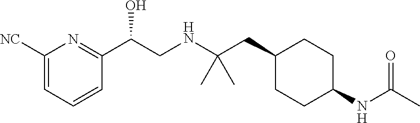 [SMILES: CC(=O)N[C@H]1CC[C@@H](CC(C)(C)NC[C@@H](O)c2cccc(C#N)n2)CC1] | N-[(1S,4s)-4-{2-[(R)-2- (6-cyano-2-pyridyl)-2- hydroxyethylamino]-2- methylpropyl} cyclohexyl]]acetamide | − | − |
| 424 | 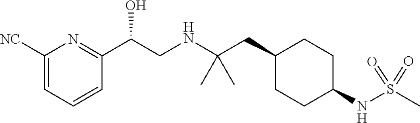 [SMILES: CC(C)(C[C@H]1CC[C@@H](NS(C)(=O)=O)CC1)NC[C@@H](O)c1cccc(C#N)n1] | 6-[(R)-2-{1,1-dimethyl- 2-[(1s,4S)-4- (mesylamino)cyclohexyl ]ethylamino}-1- hydroxyethyl]-2- pyridinecarbonitrile | + | − |
| 425 | 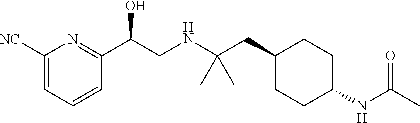 [SMILES: CC(=O)N[C@H]1CC[C@H](CC(C)(C)NC[C@H](O)c2cccc(C#N)n2)CC1] | N-[(1S,4r)-4-{2-[(S)-2- (6-cyano-2-pyridyl)-2- hydroxyethylamino]-2- methylpropyl}cyclohexy l]]acetamide | +++ | + |
| 426 | 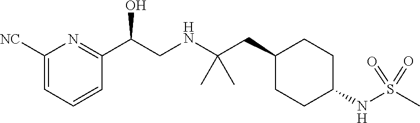 [SMILES: CC(C)(C[C@H]1CC[C@H](NS(C)(=O)=O)CC1)NC[C@H](O)c1cccc(C#N)n1] | 6-[(S)-2-{1, 1-dimethyl- 2-[(1r,4S)-4- (mesylamino)cyclohexyl] ethylamino}-1- hydroxyethyl]-2- pyridinecarbonitrile | +++ | − |
| 427 | 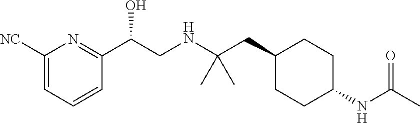 [SMILES: CC(=O)N[C@H]1CC[C@H](CC(C)(C)NC[C@@H](O)c2cccc(C#N)n2)CC1] | N-[(1R,4r)-4-{2-[(R)-2- (6-cyano-2-pyridyl)-2- hydroxyethylamino]-2- methylpropyl}cyclohexyl]] acetamide | − | − |
| 428 | 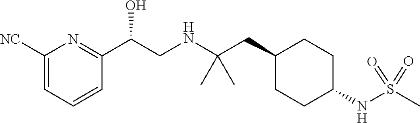 [SMILES: CC(C)(C[C@H]1CC[C@H](NS(C)(=O)=O)CC1)NC[C@@H](O)c1cccc(C#N)n1] | 6-[(R)-2-{1,1-dimethyl- 2-[(1r,4R)-4- (mesylamino)cyclohexyl] ethylamino}-1- hydroxyethyl]-2- pyridinecarbonitrile | − | − |
| 429 | 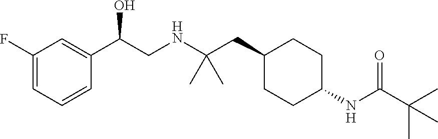 [SMILES: CC(C)(C[C@H]1CC[C@H](NC(=O)C(C)(C)C)CC1)NC[C@H](O)c1cccc(F)c1] | N-[(1R,4r)-4-{2-[(R)-2- (m-fluorophenyl)-2- hydroxyethylamino]-2- methylpropyl}cyclohexyl] ]2,2- dimethylpropionamide | +++ | + |
| 430 | 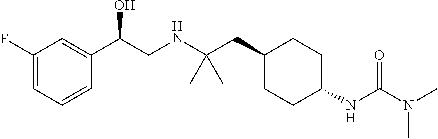 [SMILES: CN(C)C(=O)N[C@H]1CC[C@H](CC(C)(C)NC[C@H](O)c2cccc(F)c2)CC1] | (R)-2-{1,1-dimethyl-2- [(1r,4R)-4-(3,3- dimethylureido)cyclohexyl] ethylamino}-1-(m- fluorophenyl)-1-ethanol | +++ | ++ |
| 431 | 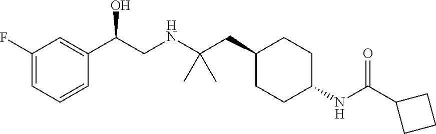 [SMILES: CC(C)(C[C@H]1CC[C@H](NC(=O)C2CCC2)CC1)NC[C@H](O)c1cccc(F)c1] | N-[(1R,4r)-4-{2-[(R)-2- (m-fluorophenyl)-2- hydroxyethylamino]-2- methylpropyl}cyclohexyl] ]cyclobutanecarboxamide | +++ | ++ |
| 432 | 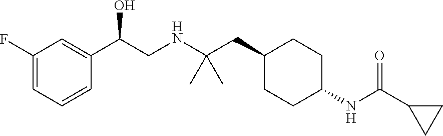 [SMILES: CC(C)(C[C@H]1CC[C@H](NC(=O)C2CC2)CC1)NC[C@H](O)c1cccc(F)c1] | N-[(1R,4r)-4-{2-[(R)-2- (m-fluorophenyl)-2- hydroxyethylamino]-2- methylpropyl}cyclohexyl]] cyclopropanecarboxamide | +++ | +++ |
| 433 | 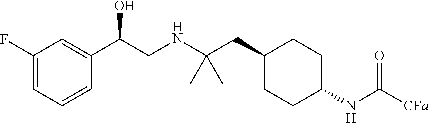 | N-[(1R,4r)-4-{2-[(R)-2- (m-fluorophenyl)-2- hydroxyethylamino]-2- methylpropyl}cyclohexyl]] trifluoroacetamide | +++ | + |
| 434 | 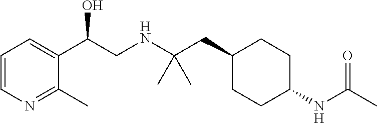 [SMILES: CC(=O)N[C@H]1CC[C@H](CC(C)(C)NC[C@H](O)c2cccnc2C)CC1] | N-[(1R,4r)-4-{2-[(R)-2- hydroxy-2-(2-methyl-3- pyridyl)ethylamino]-2- methylpropyl}cyclohexyl]] acetamide | +++ | − |
| 435 | 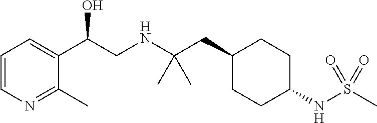 [SMILES: Cc1ncccc1[C@@H](O)CNC(C)(C)C[C@H]1CC[C@H](NS(C)(=O)=O)CC1] | (R)-2-{1,1-dimethyl-2- [(1r,4R)-4- (mesylamino)cyclohexyl] ethylamino}-1-(2- methyl-3-pyridyl)-1- ethanol | + | − |
| 436 | 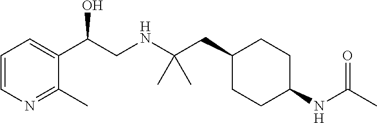 [SMILES: CC(=O)N[C@H]1CC[C@@H](CC(C)(C)NC[C@H](O)c2cccnc2C)CC1] | N-[(1S,4s)-4-{2-[(R)-2- hydroxy-2-(2-methyl-3- pyridyl)ethylamino]-2- methylpropyl}cyclohexyl]] acetamide | ++ | − |
| 437 | 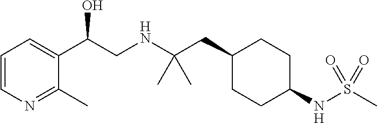 [SMILES: Cc1ncccc1[C@@H](O)CNC(C)(C)C[C@H]1CC[C@@H](NS(C)(=O)=O)CC1] | (R)-2-{1,1-dimethyl-2- [(1s,4S)-4- (mesylamino)cyclohexyl] ethylamino}-1-(2- methyl-3-pyridyl)-1- ethanol | + | − |
| 438 | 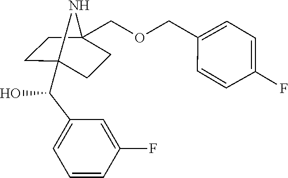 [SMILES: O[C@@H](c1cccc(F)c1)C12CCC(COCc3ccc(F)cc3)(CC1)N2] | (S)-(m-fluorophenyl)(4- {[(p- fluorophenyl)methoxy] methyl}-7- azabicyclo[[2.2.1]]hept- 1-yl)methanol | − | − |
| 439 | 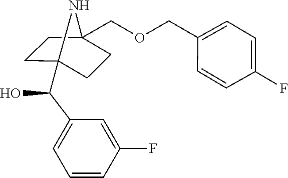 [SMILES: O[C@H](c1cccc(F)c1)C12CCC(COCc3ccc(F)cc3)(CC1)N2] | (R)-(m-fluorophenyl)(4- {[(p- fluorophenyl)methoxy] methyl}-7- azabicyclo[[2.2.1]]hept- 1-yl)methanol | +++ | + |
| 440 | 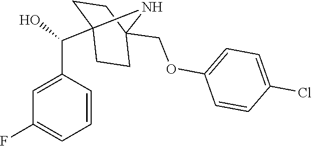 [SMILES: O[C@@H](c1cccc(F)c1)C12CCC(COc3ccc(Cl)cc3)(CC1)N2] | (S)-{4-[(p- chlorophenoxy)methyl]- 7- azabicyclo[[2.2.1]hept- 1-yl}(m- fluorophenyl)methanol | +++ | − |
| 441 | 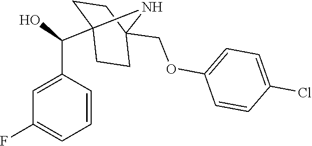 [SMILES: O[C@H](c1cccc(F)c1)C12CCC(COc3ccc(Cl)cc3)(CC1)N2] | (R)-{4-[(p- chlorophenoxy)methyl]- 7- azabicyclo[[2.2.1]]hept- 1-yl}(m- fluorophenyl)methanol | +++ | − |
| 442 | 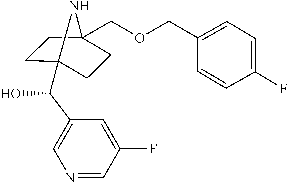 [SMILES: O[C@@H](c1cncc(F)c1)C12CCC(COCc3ccc(F)cc3)(CC1)N2] | (S)-(4-{[(p- fluorophenyl)methoxy] methyl}-7- azabicyclo[[2.2.1]]hept- 1-yl)(5-fluoro-3- pyridyl)methanol | + | − |
| 443 | 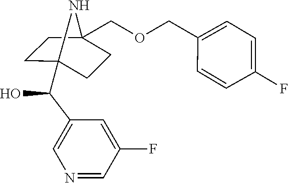 [SMILES: O[C@H](c1cncc(F)c1)C12CCC(COCc3ccc(F)cc3)(CC1)N2] | (R)-(4-{[(p- fluorophenyl)methoxy] methyl}-7- azabicyclo[[2.2.1]]hept- 1-yl)(5-fluoro-3- pyridyl)methanol | +++ | + |
| 444 | 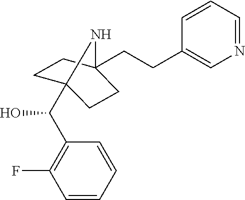 [SMILES: O[C@@H](c1ccccc1F)C12CCC(CCc3cccnc3)(CC1)N2] | (R)-(o-fluorophenyl){4- [2-(3-pyridyl)ethyl]-7- azabicyclo[[2.2.1]]hept- 1-yl}methanol | +++ | ++ |
| 445 | 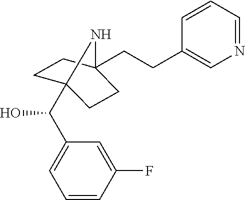 [SMILES: O[C@@H](c1cccc(F)c1)C12CCC(CCc3cccnc3)(CC1)N2] | (S)-(m-fluorophenyl){4- [2-(3-pyridyl)ethyl]-7- azabicyclo[[2.2.1]]hept- 1-yl}methanol | +++ | − |
| 446 | 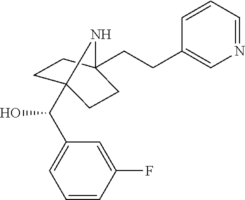 [SMILES: O[C@@H](c1cccc(F)c1)C12CCC(CCc3cccnc3)(CC1)N2] | (R)-(m-fluorophenyl){4- [2-(3-pyridyl)ethyl]-7- azabicyclo[[2.2.1]]hept- 1-yl}methanol | +++ | ++ |
| 447 | 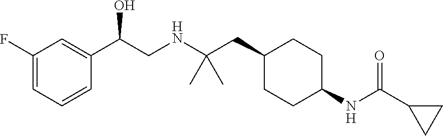 [SMILES: CC(C)(C[C@H]1CC[C@@H](NC(=O)C2CC2)CC1)NC[C@H](O)c1cccc(F)c1] | N-[(1S,4s)-4-{2-[(R)-2- (m-fluorophenyl)-2- hydroxyethylamino]-2- methylpropyl}cyclohexyl] ]cyclopropanecarboxamide | +++ | − |
| 448 | 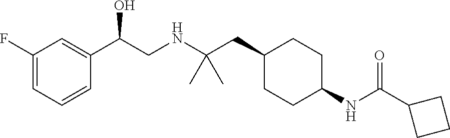 [SMILES: CC(C)(C[C@H]1CC[C@@H](NC(=O)C2CCC2)CC1)NC[C@H](O)c1cccc(F)c1] | N-[(1S,4s)-4-{2-[(R)-2- (m-fluorophenyl)-2- hydroxyethylamino]-2- methylpropyl}cyclohexyl] ]cyclobutanecarboxamide | ++ | − |
| 449 | 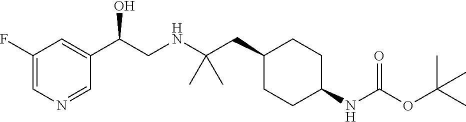 [SMILES: CC(C)(C[C@H]1CC[C@@H](NC(=O)OC(C)(C)C)CC1)NC[C@H](O)c1cncc(F)c1] | (1S,4s)-4-{2-[(R)-2- (m-fluorophenyl)-2- hydroxyethylamino]-2- methylpropyl}cyclohexy- 2-methyl-2- propanecarbamate | ++ | − |
| 450 | 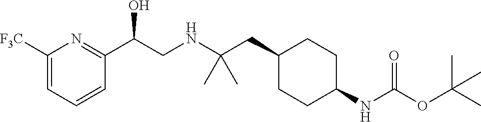 [SMILES: CC(C)(C[C@H]1CC[C@@H](NC(=O)OC(C)(C)C)CC1)NC[C@H](O)c1cccc(C(F)(F)F)n1] | (1R,4s)-4-(2-{(S)-2- hydroxy-2-[6- (trifluoromethyl)-2- pyridyl]]ethylamino}-2- methylpropyl)cyclohexyl 2-methyl-2- propanecarbamate | − | − |
| 451 | 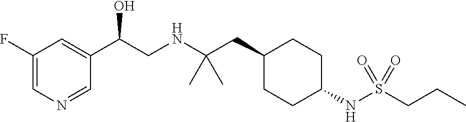 [SMILES: CCCS(=O)(=O)N[C@H]1CC[C@H](CC(C)(C)NC[C@H](O)c2cncc(F)c2)CC1] | (R)-2-{1,1-dimethyl-2- [(1r,4R)-4- (propylsulfonylamino) cyciohexyl]]ethylamino}-1- (m-fluorophenyl)-1- ethanol | ++ | + |
| 452 | 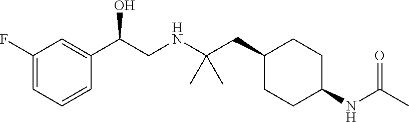 [SMILES: CC(=O)N[C@H]1CC[C@@H](CC(C)(C)NC[C@H](O)c2cccc(F)c2)CC1] | N-[(1S,4s)-4-{2-[(R)-2- (m-fluorophenyl)-2- hydroxyethylamino]-2- methylpropyl} cyclohexyl]]acetamide | +++ | − |
| 453 | 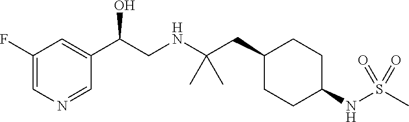 [SMILES: CC(C)(C[C@H]1CC[C@@H](NS(C)(=O)=O)CC1)NC[C@H](O)c1cncc(F)c1] | (R)-2-{1,1-dimethyl-2- [(1s,4S)-4- (mesylamino)cyclohexyl] ethylamino}-1-(m- fluorophenyl)-1-ethanol | +++ | − |
| 454 | 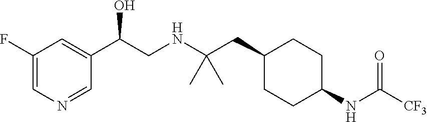 [SMILES: CC(C)(C[C@H]1CC[C@@H](NC(=O)C(F)(F)F)CC1)NC[C@H](O)c1cncc(F)c1] | N-[(1S,4s)-4-{2-[(R)-2- (m-fluorophenyl)-2- hydroxyethylamino]-2- methylpropyl}cyclohexyl]] trifluoroacetamide | +++ | − |
| 455 | 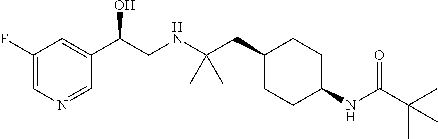 [SMILES: CC(C)(C[C@H]1CC[C@@H](NC(=O)C(C)(C)C)CC1)NC[C@H](O)c1cncc(F)c1] | N-[(1S,4s)-4-{2-[(R)-2- (m-fluorophenyl)-2- hydroxyethylamino]-2- methylpropyl} cyclohexyl]2,2- dimethylpropionamide | ++ | − |
| 456 | 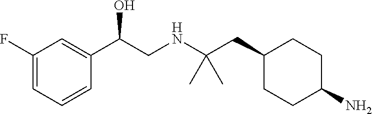 [SMILES: CC(C)(C[C@H]1CC[C@@H](N)CC1)NC[C@H](O)c1cccc(F)c1] | (R)-2-{1,1-dimethyl-2- [(1s,4S)-4- aminocyclohexyl]] ethylamino}-1-(m- fluorophenyl)-1-ethanol | +++ | − |
| 457 | 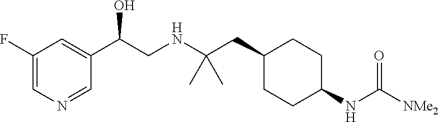 [SMILES: CN(C)C(=O)N[C@H]1CC[C@@H](CC(C)(C)NC[C@H](O)c2cncc(F)c2)CC1] | (R)-2-{1,1-dimethyl-2- [(1s,4S)-4-(3,3- dimethylureido) cyclohexyl] ethylamino}-1-(m- fluorophenyl)-1-ethanol | +++ | + |
| 458 | 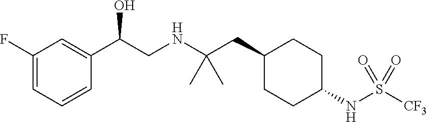 [SMILES: CC(C)(C[C@H]1CC[C@H](NS(=O)(=O)C(F)(F)F)CC1)NC[C@H](O)c1cccc(F)c1] | (R)-2-{1,1-dimethyl-2- [(1r,4R)-4- (trifluoromesylamino) cyclohexyl]]ethylamino}-1- (m-fluorophenyl)-1- ethanol | +++ | − |
| 459 | 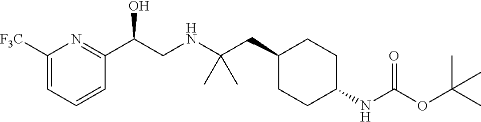 [SMILES: CC(C)(C[C@H]1CC[C@H](NC(=O)OC(C)(C)C)CC1)NC[C@H](O)c1cccc(C(F)(F)F)n1] | (1S,4r)-4-(2-{(S)-2- hydroxy-2-[6- (trifluoromethyl)-2- pyridyl]ethylamino}-2- methylpropyl)cyclohexyl 2-methyl-2- propanecarbamate | +++ | − |
| 460 | 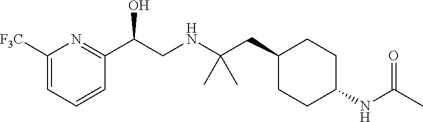 [SMILES: CC(=O)N[C@H]1CC[C@H](CC(C)(C)NC[C@H](O)c2cccc(C(F)(F)F)n2)CC1] | N-[(1S,4r)-4-(2-{(S)-2- hydroxy-2-[6- (trifluoromethyl)-2- pyridyl]]ethylamino}-2- methylpropyl)cyclohexyl] acetamide | ++ | − |
| 461 | 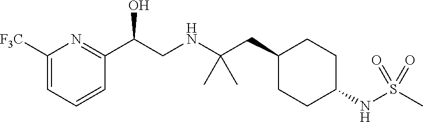 [SMILES: CC(C)(C[C@H]1CC[C@H](NS(C)(=O)=O)CC1)NC[C@H](O)c1cccc(C(F)(F)F)n1] | (S)-2-{1,1-dimethyl-2- [(1r,4S)-4- (mesylamino)cyclohexyl] ethylamino}-1-[6- (trifluoromethyl)-2- pyridyl]-1-ethanol | + | − |
| 462 | 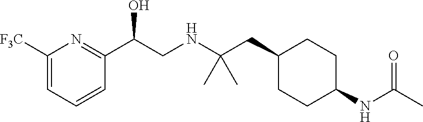 [SMILES: CC(=O)N[C@H]1CC[C@@H](CC(C)(C)NC[C@H](O)c2cccc(C(F)(F)F)n2)CC1] | N-[(1R,4s)-4-(2-{(S)-2- hydroxy-2-[6- (trifluoromethyl)-2- pyridyl]]ethylamino}-2- methylpropyl)cyclohexyl] acetamide | + | − |
| 463 | 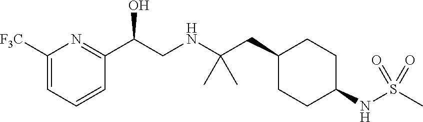 [SMILES: CC(C)(C[C@H]1CC[C@@H](NS(C)(=O)=O)CC1)NC[C@H](O)c1cccc(C(F)(F)F)n1] | (S)-2-{1,1-dimethyl-2- [(1s,4R)-4- (mesylamino)cyclohexyl] ]ethylamino}-1-[6- (trifluoromethyl)-2- pyridyl]]-1-ethanol | ++ | − |
| 464 | 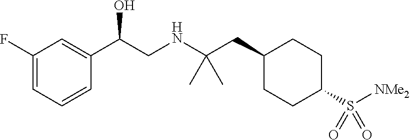 [SMILES: CN(C)S(=O)(=O)[C@H]1CC[C@H](CC(C)(C)NC[C@H](O)c2cccc(F)c2)CC1] | (R)-2-{1,1-dimethyl-2- [(1r,4R)-4- (dimethylaminosulfonyl) cyclohexyl]]ethylamino}- 1-(m-fluorophenyl)-1- ethanol | + | + |
| 465 | 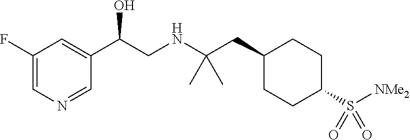 [SMILES: CN(C)S(=O)(=O)[C@H]1CC[C@H](CC(C)(C)NC[C@H](O)c2cncc(F)c2)CC1] | (R)-2-{1,1-dimethyl-2- [(1r,4R)-4- (dimethylaminosulfonyl) cyclohexyl]]ethylamino}- 1-(5-fluoro-3-pyridyl)- 1-ethanol | ++ | + |
| 466 | 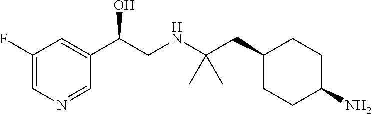 [SMILES: CC(C)(C[C@H]1CC[C@@H](N)CC1)NC[C@H](O)c1cncc(F)c1] | (R)-2-{1,1-dimethyl-2- [(1s,4S)-4- aminocyclohexyl]] ethylamino}-1-(5- fluoro-3- pyridyl)-1-ethanol | ++ | − |
| 467 | 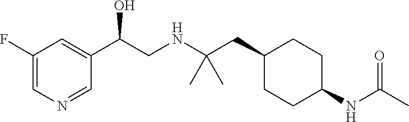 [SMILES: CC(=O)N[C@H]1CC[C@@H](CC(C)(C)NC[C@H](O)c2cncc(F)c2)CC1] | N-[(1S,4s)-4-{2-[(R)-2- (5-fluoro-3-pyridyl)-2- hydroxyethylamino]-2- methylpropyl} cyclohexyl]]acetamide | +++ | − |
| 468 | 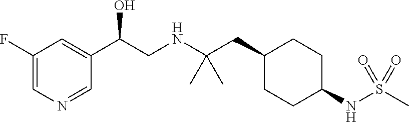 [SMILES: CC(C)(C[C@H]1CC[C@@H](NS(C)(=O)=O)CC1)NC[C@H](O)c1cncc(F)c1] | (R)-2-{1,1-dimethyl-2- [(1s,4S)-4- (mesylamino)cyclohexyl] ethylamino}-1-(5- fluoro-3-pyridyl)-1- ethanol | +++ | − |
| 469 | 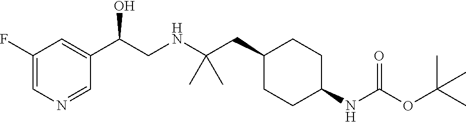 [SMILES: CC(C)(C[C@H]1CC[C@@H](NC(=O)OC(C)(C)C)CC1)NC[C@H](O)c1cncc(F)c1] | (1S,4s)-4-{2-[(R)-2-(5- fluoro-3-pyridyl)-2- hydroxyethylamino]-2- methylpropyl}cyclohexy- 2-methyl-2- propanecarbamate | +++ | − |
| 470 | 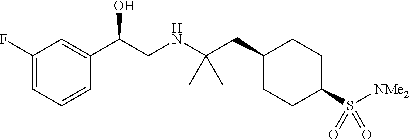 [SMILES: CN(C)S(=O)(=O)[C@H]1CC[C@@H](CC(C)(C)NC[C@H](O)c2cccc(F)c2)CC1] | (R)-2-{1,1-dimethyl-2- [(1s,4S)-4- (dimethylaminosulfonyl) cyclohexyl]]ethylamino}- 1-(m-fluorophenyl)-1- ethanol | +++ | + |
| 471 | 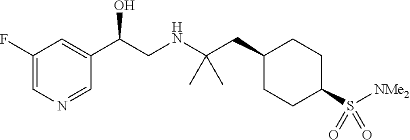 [SMILES: CN(C)S(=O)(=O)[C@H]1CC[C@@H](CC(C)(C)NC[C@H](O)c2cncc(F)c2)CC1] | (R)-2-{1,1-dimethyl-2- [(1s,4S)-4- (dimethylaminosulfonyl) cyclohexyl]]ethylamino}- 1-(5-fluoro-3-pyridyl)- 1-ethanol | +++ | ++ |
| 472 | 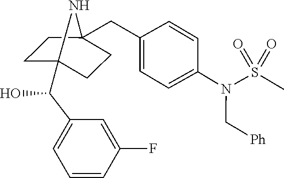 [SMILES: CS(=O)(=O)N(Cc1ccccc1)c1ccc(CC23CCC([C@@H](O)c4cccc(F)c4)(CC2)N3)cc1] | (S)-[4-({p-[(benzyl)-N- mesylamino]phenyl}me thyl)-7- azabicyclo[[2.2.1]]hept- 1-yl](m- fluorophenyl)methanol | + | − |
| 473 | 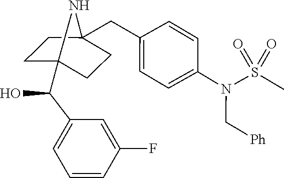 [SMILES: CS(=O)(=O)N(Cc1ccccc1)c1ccc(CC23CCC([C@H](O)c4cccc(F)c4)(CC2)N3)cc1] | (R)-[4-({p-[(benzyl)-N- mesylamino]]phenyl} methyl)-7- azabicyclo[[2.2.1]]hept- 1-yl](m- fluorophenyl)methanol | + | − |
| 474 | 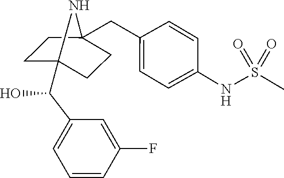 [SMILES: CS(=O)(=O)Nc1ccc(CC23CCC([C@@H](O)c4cccc(F)c4)(CC2)N3)cc1] | (S)-(m-fluorophenyl)(4- {[p- (mesylamino)phenyl] methyl}-7- azabicyclo[[2.2.1]]hept- 1-yl)methanol | ++ | − |
| 475 | 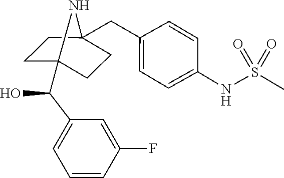 [SMILES: CS(=O)(=O)Nc1ccc(CC23CCC([C@H](O)c4cccc(F)c4)(CC2)N3)cc1] | (R)-(m-fluorophenyl)(4- {[p- (mesylamino)phenyl] methyl}-7- azabicyclo[[2.2.1]]hept- 1-yl)methanol | ++ | − |
| 476 | 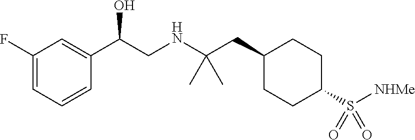 [SMILES: CNS(=O)(=O)[C@H]1CC[C@H](CC(C)(C)NC[C@H](O)c2cccc(F)c2)CC1] | (R)-2-{1,1-dimethyl-2- [(1r,4R)-4- (methylaminosulfonyl) cyclohexyl]]ethylamino}- 1-(m-fluorophenyl)-1- ethanol | +++ | + |
| 477 | 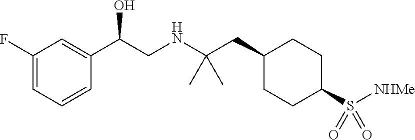 [SMILES: CNS(=O)(=O)[C@H]1CC[C@@H](CC(C)(C)NC[C@H](O)c2cccc(F)c2)CC1] | (R)-2-{1,1-dimethyl-2- [(1s,4S)-4- (methylaminosulfonyl) cyclohexyl]ethylamino}- 1-(m-fluorophenyl)-1- ethanol | ++ | − |
| 478 | 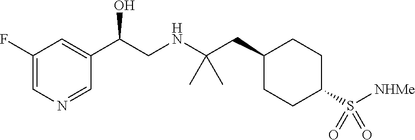 [SMILES: CNS(=O)(=O)[C@H]1CC[C@H](CC(C)(C)NC[C@H](O)c2cncc(F)c2)CC1] | (R)-2-{1,1-dimethyl-2- [(1r,4R)-4- (methylaminosulfonyl) cyclohexyl]ethylamino}- 1-(5-fluoro-3-pyridyl)- 1-ethanol | + | − |
| 479 | 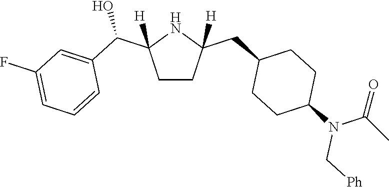 [SMILES: CC(=O)N(Cc1ccccc1)[C@H]1CC[C@@H](C[C@@H]2CC[C@H]([C@@H](O)c3cccc(F)c3)N2)CC1] | N-[(1R,4s)-4- ({(2S,5R)-5-[(S)-(m- fluorophenyl) hydroxymethyl]-2- pyrrolidinyl}methyl) cyclohexyl]-N- benzylacetamide | − | − |
| 480 | 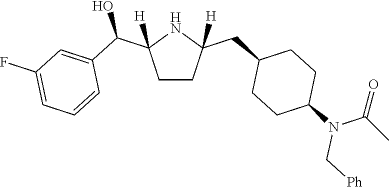 [SMILES: CC(=O)N(Cc1ccccc1)[C@H]1CC[C@@H](C[C@@H]2CC[C@H]([C@H](O)c3cccc(F)c3)N2)CC1] | N-[(1R,4s)-4- ({(2S,5R)-5-[(R)-(m- fluorophenyl) hydroxymethyl]-2- pyrrolidinyl}methyl) cyclohexyl]-N- benzylacetamide | − | − |
| 481 | 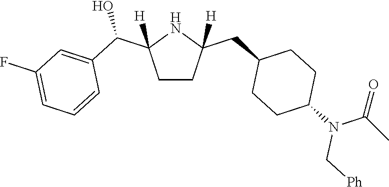 [SMILES: CC(=O)N(Cc1ccccc1)[C@H]1CC[C@H](C[C@@H]2CC[C@H]([C@@H](O)c3cccc(F)c3)N2)CC1] | N-[(1S,4r)-4-({(2S,5R)- 5-[(S)-(m- fluorophenyl) hydroxymethyl]-2- pyrrolidinyl}methyl) cyclohexyl]-N- benzylacetamide | − | − |
| 482 | 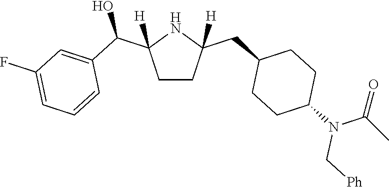 [SMILES: CC(=O)N(Cc1ccccc1)[C@H]1CC[C@H](C[C@@H]2CC[C@H]([C@H](O)c3cccc(F)c3)N2)CC1] | N-[(1S,4r)-4-({(2S,5R)- 5-[(R)-(m- fluorophenyl) hydroxymethyl]-2- pyrrolidinyl}methyl) cyclohexyl]-N- benzylacetamide | − | − |
| 483 | 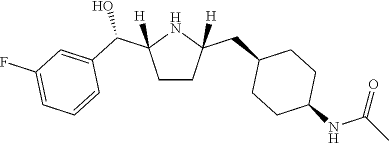 [SMILES: CC(=O)N[C@H]1CC[C@@H](C[C@@H]2CC[C@H]([C@@H](O)c3cccc(F)c3)N2)CC1] | N-[(1R,4s)-4- ({(2S,5R)-5-[(S)-(m- fluorophenyl) hydroxymethyl]-2- pyrrolidinyl}methyl) cyclohexyl]]acetamide | − | − |
| 484 | 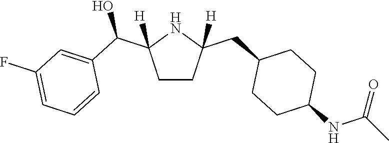 [SMILES: CC(=O)N[C@H]1CC[C@@H](C[C@@H]2CC[C@H]([C@H](O)c3cccc(F)c3)N2)CC1] | N-[(1R,4s)-4- ({(2S,5R)-5-[(R)-(m- fluorophenyl) hydroxymethyl]-2- pyrrolidinyl}methyl) cyclohexyl]]acetamide | ++ | − |
| 485 | 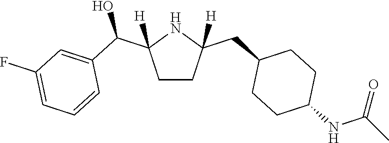 [SMILES: CC(=O)N[C@H]1CC[C@H](C[C@@H]2CC[C@H]([C@H](O)c3cccc(F)c3)N2)CC1] | N-[(1S,4r)-4-({(2S,5R)- 5-[(R)-(m- fluorophenyl) hydroxymethyl]-2- pyrrolidinyl}methyl) cyclohexyl]]acetamide | ++ | − |
| 486 | 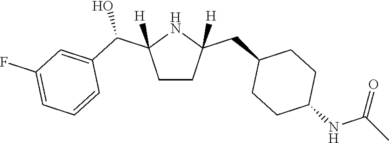 [SMILES: CC(=O)N[C@H]1CC[C@H](C[C@@H]2CC[C@H]([C@@H](O)c3cccc(F)c3)N2)CC1] | N-[(1S,4r)-4-({(2S,5R)- 5-[(S)-(m- fluorophenyl) hydroxymethyl]-2- pyrrolidinyl}methyl) cyclohexyl]]acetamide | ++ | − |
| 487 | 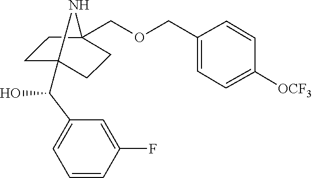 [SMILES: O[C@@H](c1cccc(F)c1)C12CCC(COCc3ccc(OC(F)(F)F)cc3)(CC1)N2] | (S)-(m-fluorophenyl)(4- {[(p- trifluoromethoxyphenyl) methoxy]]methyl}-7- azabicyclo[[2.2.1]]hept- 1-yl)methanol | − | − |
| 488 | 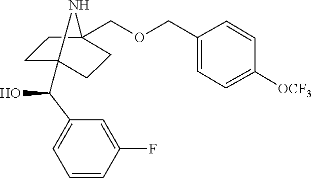 [SMILES: O[C@H](c1cccc(F)c1)C12CCC(COCc3ccc(OC(F)(F)F)cc3)(CC1)N2] | (R)-(m-fluorophenyl)(4- {[(p- trifluoromethoxyphenyl) methoxy]]methyl}-7- azabicyclo[[2.2.1]]hept- 1-yl)methanol | − | − |
| 489 | 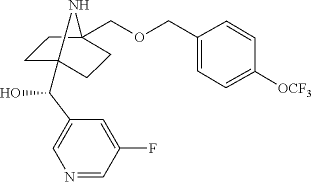 [SMILES: O[C@@H](c1cncc(F)c1)C12CCC(COCc3ccc(OC(F)(F)F)cc3)(CC1)N2] | (S)-(5-fluoro-3- pyridyl)(4-{[(p- trifluoromethoxyphenyl) methoxy]methyl}-7- azabicyclo[[2.2.1]]hept- 1-yl)methanol | − | − |
| 490 | 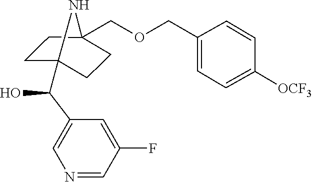 [SMILES: O[C@H](c1cncc(F)c1)C12CCC(COCc3ccc(OC(F)(F)F)cc3)(CC1)N2] | (R)-(5-fluoro-3- pyridyl)(4-{[(p- trifluoromethoxyphenyl) methoxy]]methyl}-7- azabicyclo[[2.2.1]]hept- 1-yl)methanol | ++ | − |
| 491 | 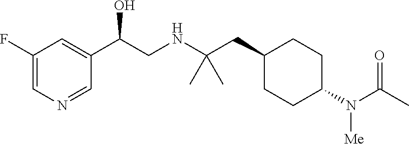 [SMILES: CC(=O)N(C)[C@H]1CC[C@H](CC(C)(C)NC[C@H](O)c2cncc(F)c2)CC1] | N-[(1R,4r)-4-{2-[(R)-2- (m-fluorophenyl)-2- hydroxyethylamino]-2- methylpropyl}cyclohexyl]]- N-methylacetamide | +++ | − |
| 492 | 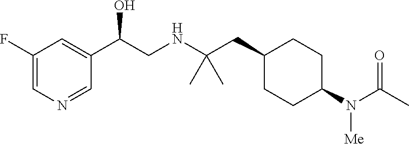 [SMILES: CC(=O)N(C)[C@H]1CC[C@@H](CC(C)(C)NC[C@H](O)c2cncc(F)c2)CC1] | N-[(1S,4s)-4-{2-[(R)-2- (m-fluorophenyl)-2- hydroxyethylamino]-2- methylpropyl}cyclohexyl]]- N-methylacetamide | + | − |
| 493 | 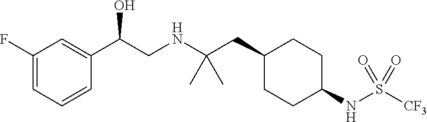 [SMILES: CC(C)(C[C@H]1CC[C@@H](NS(=O)(=O)C(F)(F)F)CC1)NC[C@H](O)c1cccc(F)c1] | (R)-2-{1,1-dimethyl-2- [(1s,4S)-4- (trifluoromesylamino) cyclohexyl]ethylamino}- 1-(m-fluorophenyl)-1- ethanol | + | − |
| 494 | 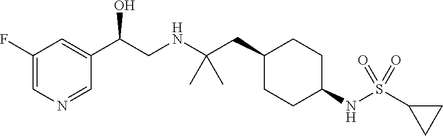 [SMILES: CC(C)(C[C@H]1CC[C@@H](NS(=O)(=O)C2CC2)CC1)NC[C@H](O)c1cncc(F)c1] | (R)-2-{1,1-dimethyl-2- [(1s,4S)-4- (cyclopropylsulfonylamino) cyclohexyl]ethylamino}- 1-(m-fluorophenyl)- 1-ethanol | + | − |
| 495 | 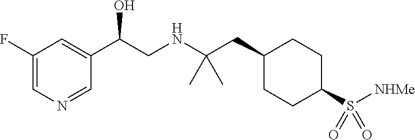 [SMILES: CNS(=O)(=O)[C@H]1CC[C@@H](CC(C)(C)NC[C@H](O)c2cncc(F)c2)CC1] | (R)-2-{1,1-dimethyl-2- [(1s,4S)-4- (methylaminosulfonyl) cyclohexyl]]ethylamino}- 1-(5-fluoro-3-pyridyl)- 1-ethanol | ++ | − |
| 496 | 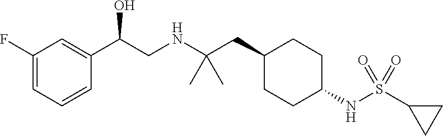 [SMILES: CC(C)(C[C@H]1CC[C@H](NS(=O)(=O)C2CC2)CC1)NC[C@H](O)c1cccc(F)c1] | (R)-2-{1,1-dimethyl-2- [(1r,4R)-4- (cyclopropylsulfonylamino) cyclohexyl]]ethylamino}- 1-(m-fluorophenyl)- 1-ethanol | ++ | − |
| 497 | 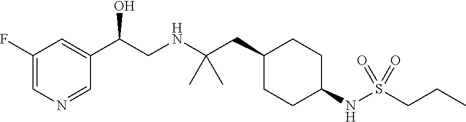 [SMILES: CCCS(=O)(=O)N[C@H]1CC[C@@H](CC(C)(C)NC[C@H](O)c2cncc(F)c2)CC1] | (R)-2-{1,1-dimethyl-2- [(1s,4S)-4- (propylsulfonylamino) cyclohexyl]]ethylamino}-1- (m-fluorophenyl)-1- ethanol | ++ | − |
| 498 | 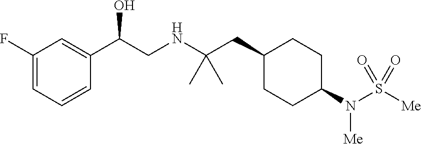 [SMILES: CN([C@H]1CC[C@@H](CC(C)(C)NC[C@H](O)c2cccc(F)c2)CC1)S(C)(=O)=O] | (R)-2-{1,1-dimethyl-2- [(1s,4S)-4-(N-mesyl-N- methylamino)cyclohexyl] ethylamino}-1-(m- fluorophenyl)-1-ethanol | + | − |
| 499 | 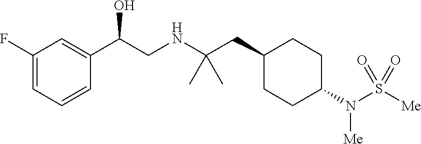 [SMILES: CN([C@H]1CC[C@H](CC(C)(C)NC[C@H](O)c2cccc(F)c2)CC1)S(C)(=O)=O] | (R)-2-{1,1-dimethyl-2- [(1r,4R)-4-(N-mesyl-N- methylamino)cyclohexyl] ethylamino}-1-(m- fluorophenyl)-1-ethanol | + | |
| 500 | 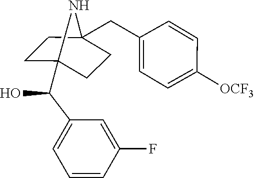 [SMILES: O[C@H](c1cccc(F)c1)C12CCC(Cc3ccc(OC(F)(F)F)cc3)(CC1)N2] | (S)-(m-fluorophenyl){4- [(p- trifluoromethoxyphenyl) methyl]-7- azabicyclo[[2.2.1]]hept- 1-yl}methanol | − | − |
| 501 | 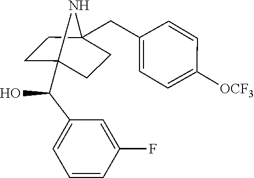 [SMILES: O[C@H](c1cccc(F)c1)C12CCC(Cc3ccc(OC(F)(F)F)cc3)(CC1)N2] | (R)-(m-fluorophenyl){4- [(p- trifluoromethoxyphenyl) methyl]-7- azabicyclo[[2.2.1]]hept- 1-yl}methanol | − | − |
| 502 | 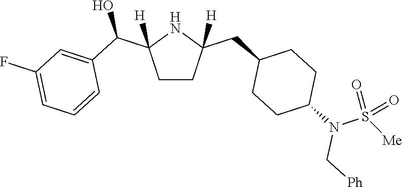 [SMILES: CS(=O)(=O)N(Cc1ccccc1)[C@H]1CC[C@H](C[C@@H]2CC[C@H]([C@H](O)c3cccc(F)c3)N2)CC1] | (R)-[(2R,5S)-5- ({(1r,4S)-4-[(benzyl)- N- mesylamino]cyclohexyl} methyl)-2- pyrrolidinyl]](m- fluorophenyl)methanol | − | − |
| 503 | 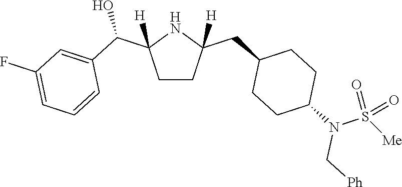 [SMILES: CS(=O)(=O)N(Cc1ccccc1)[C@H]1CC[C@H](C[C@@H]2CC[C@H]([C@@H](O)c3cccc(F)c3)N2)CC1] | (S)-[(2R,5S)-5- ({(1r,4S)-4-[(benzyl)- N- mesylamino]cyclohexyl} methyl)-2- pyrrolidinyl](m- fluorophenyl)methanol | − | − |
| 504 | 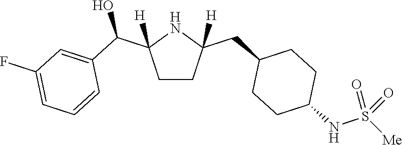 [SMILES: CS(=O)(=O)N[C@H]1CC[C@H](C[C@@H]2CC[C@H]([C@H](O)c3cccc(F)c3)N2)CC1] | (R)-[(2R,5S)-5- {[(1r,4S)-4- (mesylamino)cyclohexyl] methyl}-2- pyrrolidinyl](m- fluorophenyl)methanol] | ++ | − |
| 505 | 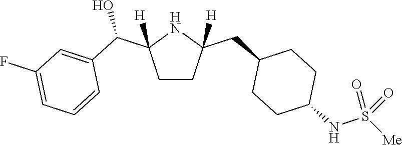 [SMILES: CS(=O)(=O)N[C@H]1CC[C@H](C[C@@H]2CC[C@H]([C@@H](O)c3cccc(F)c3)N2)CC1] | (S)-[(2R,5S)-5- {[(1r,4S)-4- (mesylamino)cyclohexyl] methyl}-2- pyrrolidinyl](m- fluorophenyl)methanol | − | − |
| 506 | 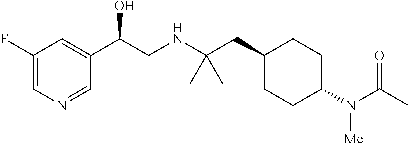 [SMILES: CC(=O)N(C)[C@H]1CC[C@H](CC(C)(C)NC[C@H](O)c2cncc(F)c2)CC1] | N-[(1R,4r)-4-{2-[(R)-2- (5-fluoro-3-pyridyl)-2- hydroxyethylamino]-2- methylpropyl}cyclohexyl]]- N-methylacetamide | +++ | + |
| 507 | 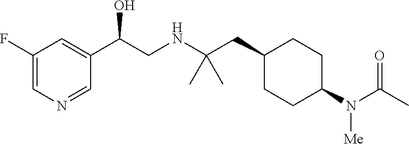 [SMILES: CC(=O)N(C)[C@H]1CC[C@@H](CC(C)(C)NC[C@H](O)c2cncc(F)c2)CC1] | N-[(1S,4s)-4-{2-[(R)-2- (5-fluoro-3-pyridyl)-2- hydroxyethylamino]-2- methylpropyl}cyclohexyl]]- N-methylacetamide | ++ | − |
| 508 | 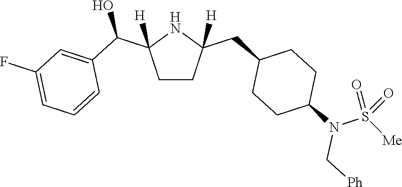 [SMILES: CS(=O)(=O)N(Cc1ccccc1)[C@H]1CC[C@@H](C[C@@H]2CC[C@H]([C@H](O)c3cccc(F)c3)N2)CC1] | (R)-[(2R,5S)-5- ({(1s,4R)-4-[(benzyl)- N- mesylamino]cyclohexyl} methyl)-2- pyrrolidinyl](m- fluorophenyl)methanol | − | − |
| 509 | 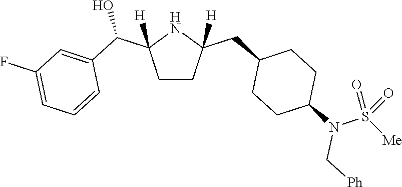 [SMILES: CS(=O)(=O)N(Cc1ccccc1)[C@H]1CC[C@@H](C[C@@H]2CC[C@H]([C@@H](O)c3cccc(F)c3)N2)CC1] | (S)-[(2R,5S)-5- ({(1s,4R)-4-[(benzyl)- N- mesylamino]cyclohexyl} methyl)-2- pyrrolidinyl](m- fluorophenyl)methanol | − | − |
| 510 | 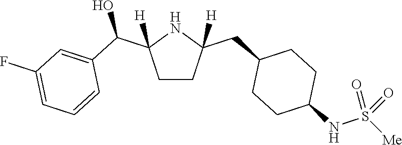 [SMILES: CS(=O)(=O)N[C@H]1CC[C@@H](C[C@@H]2CC[C@H]([C@H](O)c3cccc(F)c3)N2)CC1] | (R)-[(2R,5S)-5- {[(1s,4R)-4- (mesylamino)cyclohexyl ]methyl}-2- pyrrolidinyl](m- fluorophenyl)methanol | +++ | − |
| 511 | 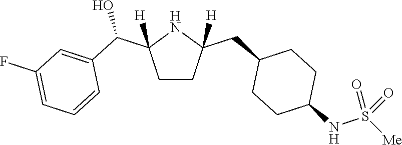 [SMILES: CS(=O)(=O)N[C@H]1CC[C@@H](C[C@@H]2CC[C@H]([C@@H](O)c3cccc(F)c3)N2)CC1] | (S)-[(2R,5S)-5- {[(1s,4R)-4- (mesylamino)cyclohexyl] methyl}-2- pyrrolidinyl]](m- fluorophenyl)methanol | + | − |
| 512 | 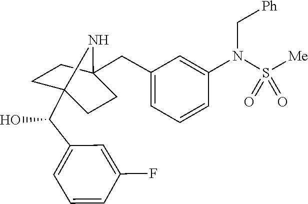 [SMILES: CS(=O)(=O)N(Cc1ccccc1)c1cccc(CC23CCC([C@@H](O)c4cccc(F)c4)(CC2)N3)c1] | (S)-[4-({m-[(benzyl)-N- mesylamino]]phenyl} methyl)-7- azabicyclo[[2.2.1]]hept- 1-yl](m- fluorophenyl)methanol | − | − |
| 513 | 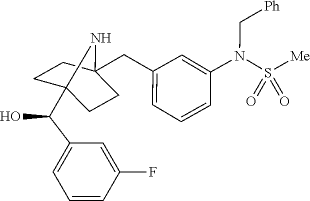 [SMILES: CS(=O)(=O)N(Cc1ccccc1)c1cccc(CC23CCC([C@H](O)c4cccc(F)c4)(CC2)N3)c1] | (R)-[4-({m-[(benzyl)-N- mesylamino]]phenyl} methyl)-7- azabicyclo[[2.2.1]]hept- 1-yl](m- fluorophenyl)methanol | + | − |
| 514 | 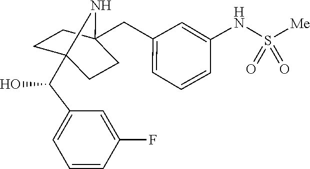 [SMILES: CS(=O)(=O)Nc1cccc(CC23CCC([C@@H](O)c4cccc(F)c4)(CC2)N3)c1] | (S)-(m-fluorophenyl)(4- {[m- (mesylamino)phenyl] methyl}-7- azabicyclo[[2.2.1]]hept- 1-yl)methanol | ++ | − |
| 515 | 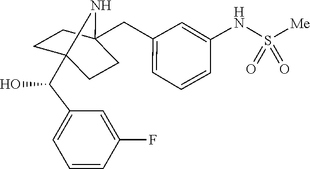 [SMILES: CS(=O)(=O)Nc1cccc(CC23CCC([C@@H](O)c4cccc(F)c4)(CC2)N3)c1] | (R)-(m-fluorophenyl)(4- {[m- (mesylamino)phenyl] methyl}-7- azabicyclo[[2.2.1]]hept- 1-yl)methanol | ++ | + |
| 516 | 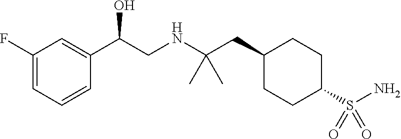 [SMILES: CC(C)(C[C@H]1CC[C@H](S(N)(=O)=O)CC1)NC[C@H](O)c1cccc(F)c1] | (1R,4r)-4-{2-[(R)-2- (m-fluorophenyl)-2- hydroxyethylamino]-2- methylpropyl} cyclohexanesulfonamide | +++ | − |
| 517 | 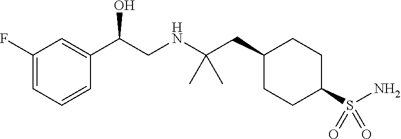 [SMILES: CC(C)(C[C@H]1CC[C@@H](S(N)(=O)=O)CC1)NC[C@H](O)c1cccc(F)c1] | (1S,4s)-4-{2-[(R)-2- (m-fluorophenyl)-2- hydroxyethylamino]-2- methylpropyl} cyclohexanesulfonamide | + | − |
| 518 | 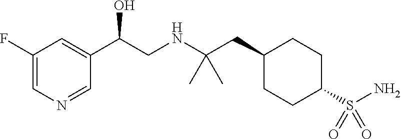 [SMILES: CC(C)(C[C@H]1CC[C@H](S(N)(=O)=O)CC1)NC[C@H](O)c1cncc(F)c1] | (1R,4r)-4-{2-[(R)-2-(5- fluoro-3-pyridyl)-2- hydroxyethylamino]-2- methylpropyl} cyclohexanesulfonamide | ++ | − |
| 519 | 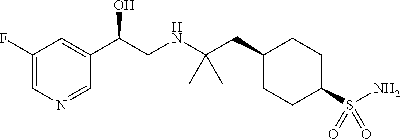 [SMILES: CC(C)(C[C@H]1CC[C@@H](S(N)(=O)=O)CC1)NC[C@H](O)c1cncc(F)c1] | (1S,4s)-4-{2-[(R)-2-(5- fluoro-3-pyridyl)-2- hydroxyethylamino]-2- methylpropyl} cyclohexanesulfonamide | +++ | − |
| 520 | 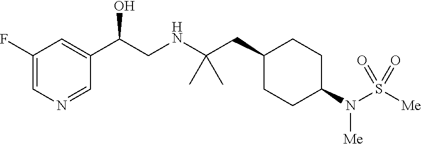 [SMILES: CN([C@H]1CC[C@@H](CC(C)(C)NC[C@H](O)c2cncc(F)c2)CC1)S(C)(=O)=O] | (R)-2-{1,1-dimethyl-2- [(1s,4S)-4-(N-mesyl-N- methylamino)cyclohexyl] ]ethylamino}-1-(5- fluoro-3-pyridyl)-1- ethanol | ++ | − |
| 521 | 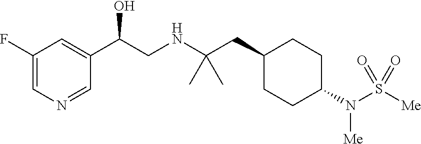 [SMILES: CN([C@H]1CC[C@H](CC(C)(C)NC[C@H](O)c2cncc(F)c2)CC1)S(C)(=O)=O] | (R)-2-{1,1-dimethyl-2- [(1r,4R)-4-(N-mesyl-N- methylamino)cyclohexyl] ethylamino}-1-(5- fluoro-3-pyridyl)-1- ethanol | ++ | − |
| 522 | 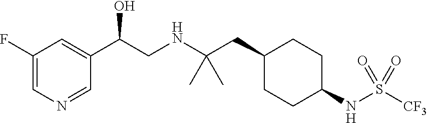 [SMILES: CC(C)(C[C@H]1CC[C@@H](NS(=O)(=O)C(F)(F)F)CC1)NC[C@H](O)c1cncc(F)c1] | (R)-2-{1,1-dimethyl-2- [(1s,4S)-4- (trifluoromesylamino) cyclohexyl]]ethylamino}-1- (5-fluoro-3-pyridyl)-1- ethanol | +++ | − |
| 523 | 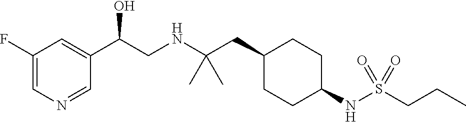 [SMILES: CCCS(=O)(=O)N[C@H]1CC[C@@H](CC(C)(C)NC[C@H](O)c2cncc(F)c2)CC1] | (R)-2-{1,1-dimethyl-2- [(1s,4S)-4- (propylsulfonylamino) cyclohexyl]ethylamino}-1- (5-fluoro-3-pyridyl)-1- ethanol | ++ | − |
| 524 | 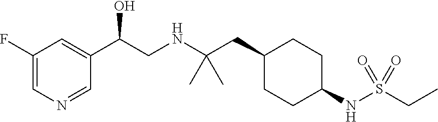 [SMILES: CCS(=O)(=O)N[C@H]1CC[C@@H](CC(C)(C)NC[C@H](O)c2cncc(F)c2)CC1] | (R)-2-{1,1-dimethyl-2- [(1s,4S)-4- (ethylsulfonylamino) cyclohexyl]]ethylamino}-1- (m-fluorophenyl)-1- ethanol | ++ | − |
| 525 | 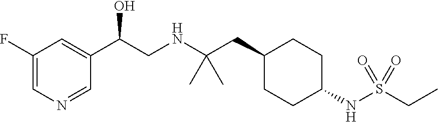 [SMILES: CCS(=O)(=O)N[C@H]1CC[C@H](CC(C)(C)NC[C@H](O)c2cncc(F)c2)CC1] | (R)-2-{1,1-dimethyl-2- [(1r,4R)-4- (ethylsulfonylamino) cyclohexyl]ethylamino}-1- (5-fluoro-3-pyridyl)-1- ethanol | ++ | − |
| 526 | 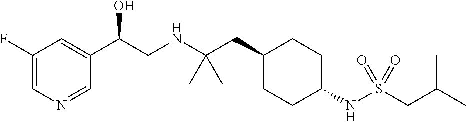 [SMILES: CC(C)CS(=O)(=O)N[C@H]1CC[C@H](CC(C)(C)NC[C@H](O)c2cncc(F)c2)CC1] | (R)-2-{1,1-dimethyl-2- [(1r,4R)-4- (isobutylsulfonylamino) cyclohexyl]]ethylamino}- 1-(5-fluoro-3-pyridyl)- 1-ethanol | ++ | − |
| 527 | 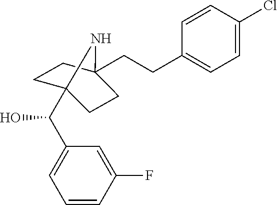 [SMILES: O[C@@H](c1cccc(F)c1)C12CCC(CCc3ccc(Cl)cc3)(CC1)N2] | (S)-{4-[2-(p- chlorophenyl)ethyl]-7- azabicyclo[[2.2.1]]hept- 1-yl}(m- fluorophenyl)methanol | − | − |
| 528 | 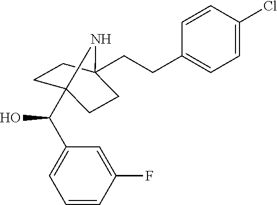 [SMILES: O[C@H](c1cccc(F)c1)C12CCC(CCc3ccc(Cl)cc3)(CC1)N2] | (R)-{4-[2-(p- chlorophenyl)ethyl]-7- azabicyclo[[2.2.1]]hept- 1-yl}(m- fluorophenyl)methanol | − | − |
| 529 | 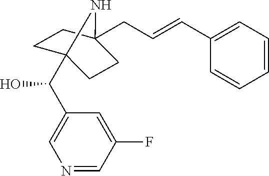 [SMILES: O[C@@H](c1cncc(F)c1)C12CCC(C/C=C/c3ccccc3)(CC1)N2] | (S)-{4-[(E)-3-phenyl-2- propenyl]-7- azabicyclo[[2.2.1]]hept- 1-yl}(5-fluoro-3- pyridyl)methanol | + | − |
| 530 | 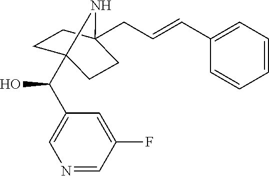 [SMILES: O[C@H](c1cncc(F)c1)C12CCC(C/C=C/c3ccccc3)(CC1)N2] | (R)-{4-[(E)-3-phenyl-2- propenyl]-7- azabicyclo[[2.2.1]]hept- 1-yl}(5-fluoro-3- pyridyl)methanol | +++ | + |
| 531 | 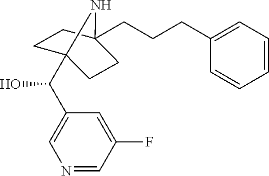 [SMILES: O[C@@H](c1cncc(F)c1)C12CCC(CCCc3ccccc3)(CC1)N2] | (S)-(5-fluoro-3- pyridyl){4-(3- phenylpropyl)-7- azabicyclo[[2.2.1]]hept- 1-yl}methanol | ++ | − |
| 532 | 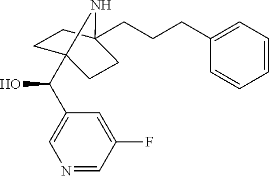 [SMILES: O[C@H](c1cncc(F)c1)C12CCC(CCCc3ccccc3)(CC1)N2] | (R)-(5-fluoro-3- pyridyl){4-(3- phenylpropyl)-7- azabicyclo[[2.2.1]]hept- 1-yl}methanol | ++ | + |
| 533 | 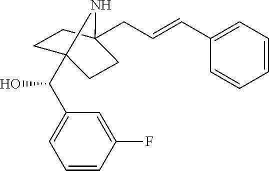 [SMILES: O[C@@H](c1cccc(F)c1)C12CCC(C/C=C/c3ccccc3)(CC1)N2] | (S)-{4-[(E)-3-phenyl-2- propenyl]-7- azabicyclo[[2.2.1]]hept- 1-yl}(m- fluorophenyl)methanol | − | − |
| 534 | 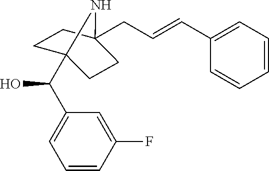 [SMILES: O[C@H](c1cccc(F)c1)C12CCC(C/C=C/c3ccccc3)(CC1)N2] | (R)-{4-[(E)-3-phenyl-2- propenyl]-7- azabicyclo[2.2.1]]hept- 1-yl}(m- fluorophenyl)methanol | +++ | − |
| 535 | 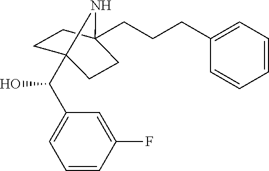 [SMILES: O[C@@H](c1cccc(F)c1)C12CCC(CCCc3ccccc3)(CC1)N2] | (S)-(m-fluorophenyl){4- (3-phenylpropyl)-7- azabicyclo[[2.2.1]]hept- 1-yl}methanol | + | − |
| 536 | 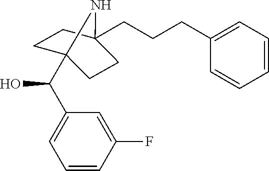 [SMILES: O[C@H](c1cccc(F)c1)C12CCC(CCCc3ccccc3)(CC1)N2] | (R)-(m-fluorophenyl){4- (3-phenylpropyl)-7- azabicyclo[[2.2.1]]hept- 1-yl}methanol | +++ | + |
| 537 | 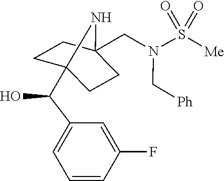 [SMILES: CS(=O)(=O)N(Cc1ccccc1)CC12CCC([C@H](O)c3cccc(F)c3)(CC1)N2] | (R)-(4-{[(benzyl)-N- mesylamino]methyl}-7- azabicyclo[[2.2.1]]hept- 1-yl)(m- fluorophenyl)methanol | ++ | + |
| 538 | 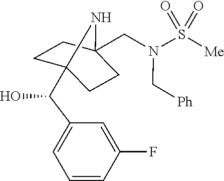 [SMILES: CS(=O)(=O)N(Cc1ccccc1)CC12CCC([C@@H](O)c3cccc(F)c3)(CC1)N2] | (S)-(4-{[(benzyl)-N- mesylamino]methyl}-7- azabicyclo[[2.2.1]]hept- 1-yl)(m- fluorophenyl)methanol | ++ | − |
| 539 | 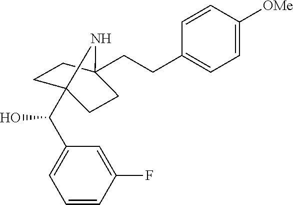 [SMILES: COc1ccc(CCC23CCC([C@@H](O)c4cccc(F)c4)(CC2)N3)cc1] | (S)-(m-fluorophenyl){4- [2-(p- methoxyphenyl)ethyl]- 7- azabicyclo[[2.2.1]]hept- 1-yl}methanol | − | − |
| 540 | 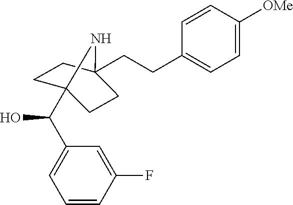 [SMILES: COc1ccc(CCC23CCC([C@H](O)c4cccc(F)c4)(CC2)N3)cc1] | (R)-(m-fluorophenyl){4- [2-(p- methoxyphenyl)ethyl]- 7- azabicyclo[[2.2.1]]hept- 1-yl}methanol | ++ | + |
| 541 | 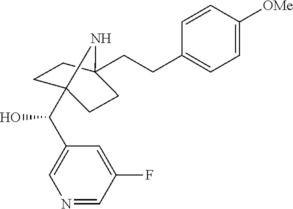 [SMILES: COc1ccc(CCC23CCC([C@@H](O)c4cncc(F)c4)(CC2)N3)cc1] | (S)-(5-fluoro-3- pyridyl){4-[2-(p- methoxyphenyl)ethyl]- 7- azabicyclo[[2.2.1]]hept- 1-yl}methanol | − | − |
| 542 | 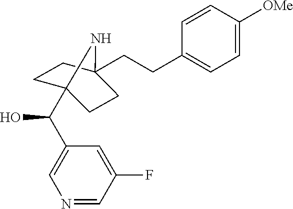 [SMILES: COc1ccc(CCC23CCC([C@H](O)c4cncc(F)c4)(CC2)N3)cc1] | (R)-(5-fluoro-3- pyridyl){4-[2-(p- methoxyphenyl)ethyl]- 7- azabicyclo[[2.2.1]]hept- 1-yl}methanol | ++ | + |
| 543 | 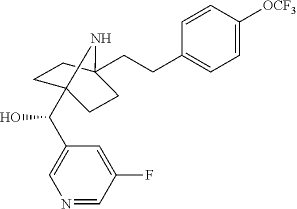 [SMILES: O[C@@H](c1cncc(F)c1)C12CCC(CCc3ccc(OC(F)(F)F)cc3)(CC1)N2] | (S)-(5-fluoro-3- pyridyl){4-[2-(p- trifluoromethoxyphenyl) ethyl]-7- azabicyclo[[2.2.1]]hept- 1-yl}methanol | − | − |
| 544 | 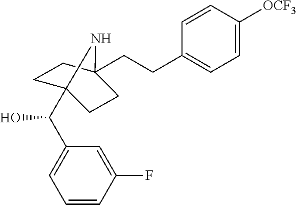 [SMILES: O[C@@H](c1cccc(F)c1)C12CCC(CCc3ccc(OC(F)(F)F)cc3)(CC1)N2] | (S)-(m-fluorophenyl){4- [2-(p- trifluoromethoxyphenyl) ethyl]-7- azabicyclo[[2.2.1]]hept- 1-yl}methanol | − | − |
| 545 | 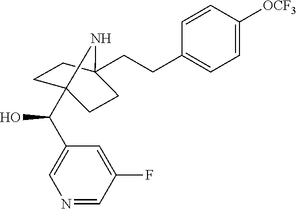 [SMILES: O[C@H](c1cncc(F)c1)C12CCC(CCc3ccc(OC(F)(F)F)cc3)(CC1)N2] | (R)-(5-fluoro-3- pyridyl){4-[2-(p- trifluoromethoxyphenyl) ethyl]-7- azabicyclo[[2.2.1]]hept- 1-yl}methanol | − | − |
| 546 | 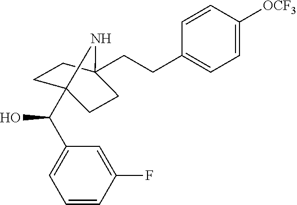 [SMILES: O[C@H](c1cccc(F)c1)C12CCC(CCc3ccc(OC(F)(F)F)cc3)(CC1)N2] | (R)-(m-fluorophenyl){4- [2-(p- trifluoromethoxyphenyl) ethyl]-7- azabicyclo[[2.2.1]]hept- 1-yl}methanol | − | − |
| 547 | 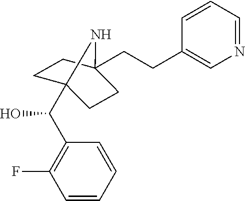 [SMILES: O[C@@H](c1ccccc1F)C12CCC(CCc3cccnc3)(CC1)N2] | (S)-(o-fluorophenyl){4- [2-(3-pyridyl)ethyl]-7- azabicyclo[[2.2.1]]hept- 1-yl}methanol | +++ | - |

In particular embodiments, the β₂-adrenergic receptor agonist is selected from the group consisting of:
2-(butylamino)-1-(p-hydroxyphenyl)-1-ethanol;
2-(tert-butylamino)-1-(p-hydroxyphenyl)-1-ethanol;
2-[(cyclohexylmethyl)amino]-1-(p-hydroxyphenyl)-1-ethanol;
(R)-2-(butylamino)-1-(p-hydroxyphenyl)-1-ethanol;
1-(m-hydroxyphenyl)-2-(1-methylbutylamino)-1-ethanol;
2-(3-cyclohexylpropylamino)-1-(p-hydroxyphenyl)-1-ethanol;
(R)-2-[(R)-1-methylbutylamino]-1-(p-hydroxyphenyl)-1-ethanol;
1-(4-amino-3,5-dichlorophenyl)-2-(butylamino)-1-ethanol;
2-(3-cyclopropylpropylamino)-1-(p-hydroxyphenyl)-1-ethanol;
1-[4-amino-3-chloro-5-(trifluoromethyl)phenyl]-2-(butylamino)-1-ethanol;
2-(butylamino)-1-(4-chloro-3-hydroxyphenyl)-1-ethanol;
1-(4-chloro-3-hydroxyphenyl)-2-(1-methylbutylamino)-1-ethanol;
m-{(R)-[(2R,5R)-5-methyl-2-pyrrolidinyl]hydroxymethyl}phenol and m-{(S)-[(2S,5S)-5-methyl-2-pyrrolidinyl]hydroxymethyl}phenol;
1-(3-amino-2,4-difluorophenyl)-2-(butylamino)-1-ethanol;
(R)-[(2R,6R)-6-propyl-2-piperidyl](m-hydroxyphenyl)methanol;
(R)-2-[(R)-1-methylbutylamino]-1-(m-fluorophenyl)-1-ethanol;
(R)-2-[(S)-1-methylbutylamino]-1-(m-fluorophenyl)-1-ethanol;
(R)-2-(tert-butylamino)-1-(m-fluorophenyl)-1-ethanol;
(R)-[(2R,5R)-5-propyl-2-pyrrolidinyl](m-fluorophenyl)methanol;
(R)-2-(1,1-dimethylbutylamino)-1-(m-fluorophenyl)-1-ethanol;
(R)-[(2R,6S)-6-propyl-2-piperidyl](m-hydroxyphenyl)methanol;
(R)-2-(tert-butylamino)-1-(2,3-difluorophenyl)-1-ethanol;
(R)-2-(butylamino)-1-(2,3-difluorophenyl)-1-ethanol;
(R)-2-(tert-butylamino)-1-(o-fluorophenyl)-1-ethanol;
(R)-2-(butylamino)-1-(o-fluorophenyl)-1-ethanol;
(R)-1-(m-fluorophenyl)-2-(1-methylcyclopropylamino)-1-ethanol;
(R)-1-(m-fluorophenyl)-2-(1-methylcyclobutylamino)-1-ethanol;
(R)-2-(butylamino)-1-(4-fluoro-3-hydroxyphenyl)-1-ethanol;
(R)-2-(butylamino)-1-(2-fluoro-3-hydroxyphenyl)-1-ethanol;
(R)-1-(3-amino-2-fluorophenyl)-2-(tert-butylamino)-1-ethanol;
4-[(R)-2-(tert-butylamino)-1-hydroxyethyl]-1-methyl-2(1H)-pyridinone;
(R)-2-(tert-butylamino)-1-(5-fluoro-3-pyridyl)-1-ethanol;
5-[(R)-2-(tert-butylamino)-1-hydroxyethyl]-3-pyridinol;
(R)-[(R)-5,5-dimethyl-2-pyrrolidinyl](m-fluorophenyl)methanol;
m-{(R)-[(R)-5,5-dimethyl-2-pyrrolidinyl]hydroxymethyl}phenol;
(R)-[(2S,6R)-6-propyl-2-piperidyl](m-chlorophenyl)methanol;
(S)-[(2S,6S)-6-propyl-2-piperidyl](m-hydroxyphenyl)methanol;
4-[2-(tert-butylamino)-1-hydroxyethyl]-3-pyridinol;
(R)-[(2S,6R)-6-propyl-2-piperidyl](m-hydroxyphenyl)methanol;
(R)-[(2R,5R)-5-propyl-2-pyrrolidinyl](o-chlorophenyl)methanol;
(R)-1-(3-amino-2,4-difluorophenyl)-2-(tert-butylamino)-1-ethanol;
(R)-2-(tert-butylamino)-1-(3-fluoro-2-tolyl)-1-ethanol;
(R)-[(R)-5,5-dimethyl-2-pyrrolidinyl](o-chlorophenyl)methanol;
(R)-[(R)-5,5-dimethyl-2-pyrrolidinyl](m-chlorophenyl)methanol;
(R)-[(R)-5,5-dimethyl-2-pyrrolidinyl](5-fluoro-3-pyridyl)methanol;
(R)-[(2R,5R)-5-propyl-2-pyrrolidinyl](5-fluoro-3-pyridyl)methanol;
(S)-[(R)-6,6-dimethyl-2-piperidyl](o-chlorophenyl)methanol;
(R)-[(R)-5,5-dimethyl-2-pyrrolidinyl](o-fluorophenyl)methanol;
(S)-[(R)-6,6-dimethyl-2-piperidyl](o-fluorophenyl)methanol;
(S)-(o-fluorophenyl)(4-methyl-7-azabicyclo[2.2.1]hept-1-yl)methanol;
(R)-(o-fluorophenyl)(4-methyl-7-azabicyclo[2.2.1]hept-1-yl)methanol;
(R)-(5-fluoro-3-pyridyl)(4-methyl-7-azabicyclo[2.2.1]hept-1-yl)methanol;
(R)-[(S)-6,6-dimethyl-2-piperidyl](m-fluorophenyl)methanol;
(R)-[(S)-6,6-dimethyl-2-piperidyl](o-fluorophenyl)methanol;
(R)-(o-chlorophenyl)(4-methyl-7-azabicyclo[2.2.1]hept-1-yl)methanol;
(R)-(m-chlorophenyl)(4-methyl-7-azabicyclo[2.2.1]hept-1-yl)methanol;
(R)-[(R)-4,4-dimethyl-2-azetidinyl](m-fluorophenyl)methanol;
(R)-[(R)-4,4-dimethyl-2-azetidinyl](o-fluorophenyl)methanol;
(R)-{(R)-1-methyl-2-azabicyclo[2.1.1]hex-3-yl}(m-chlorophenyl)methanol;
(S)-{(S)-1-methyl-2-azabicyclo[2.1.1]hex-3-yl}(o-fluorophenyl)methanol;
(S)-{(R)-1-methyl-2-azabicyclo[2.1.1]hex-3-yl}(o-fluorophenyl)methanol;
(R)-{(S)-1-methyl-2-azabicyclo[2.1.1]hex-3-yl}(o-fluorophenyl)methanol;
(R)-[(R)-4,4-dimethyl-2-azetidinyl](5-fluoro-3-pyridyl)methanol;
(R)-{(R)-1-methyl-2-azabicyclo[2.1.1]hex-3-yl}(5-fluoro-3-pyridyl)methanol;
(R)-[(R)-5,5-dimethyl-2-pyrrolidinyl](3-amino-2-fluorophenyl)methanol;
(R)-[(R)-4,4-dimethyl-2-azetidinyl](m-chlorophenyl)methanol;
(R)-[(2S,7S)-7-propyl-2-azepanyl](5-fluoro-3-pyridyl)methanol;
(S)-[(R)-5,5-dimethyl-2-pyrrolidinyl](4-amino-3,5-difluorophenyl)methanol;
(R)-1-(5-fluoro-3-pyridyl)-2-{2-[(1s,4S)-4-methoxycyclohexyl]ethylamino}-1-ethanol;
(R)-1-(m-fluorophenyl)-2-{1-methyl-1-[(1r,4R)-4-methoxycyclohexyl]ethylamino}-1-ethanol;
(R)-1-(5-fluoro-3-pyridyl)-2-{1-methyl-1-[(1r,4R)-4-methoxycyclohexyl]ethylamino}-1-ethanol;
(R)-{(R)-1-methyl-2-azabicyclo[2.1.1]hex-3-yl}(3-amino-2-fluorophenyl)methanol;
(R)-2-{1,1-dimethyl-2-[(1s,4S)-4-methoxycyclohexyl]ethylamino}-1-(m-fluorophenyl)-1-ethanol;
(R)-2-{1,1-dimethyl-2-[(1s,4S)-4-methoxycyclohexyl]ethylamino}-1-(5-fluoro-3-pyridyl)-1-ethanol;
(R)-2-{1,1-dimethyl-2-[(1r,4R)-4-methoxycyclohexyl]ethylamino}-1-(m-fluorophenyl)-1-ethanol;
(R)-2-{1-[(1S,3R)-3-methoxycyclohexyl]-1-methylethylamino}-1-(m-fluorophenyl)-1-ethanol;
(R)-2-{1-[(1R,3S)-3-methoxycyclohexyl]-1-methylethylamino}-1-(m-fluorophenyl)-1-ethanol;
(R)-1-(5-fluoro-3-pyridyl)-2-{3-[(1s,4R)-4-methoxycyclohexyl]propylamino}-1-ethanol;
(R)-1-(5-fluoro-3-pyridyl)-2-({[(1s,4S)-4-(benzyloxy)cyclohexyl]methyl}amino)-1-ethanol;
(R)-2-{1,1-dimethyl-3-[(1s,4R)-4-methoxycyclohexyl]propylamino}-1-(m-fluorophenyl)-1-ethanol;
(R)-2-{1,1-dimethyl-3-[(1s,4R)-4-methoxycyclohexyl]propylamino}-1-(5-fluoro-3-pyridyl)-1-ethanol;
(R)-1-(m-fluorophenyl)-2-{1-methyl-1-[(1s,4S)-4-(benzyloxy)cyclohexyl]ethylamino}-1-ethanol;
(R)-2-{1,1-dimethyl-3-[(1s,4R)-4-hydroxycyclohexyl]propylamino}-1-(m-fluorophenyl)-1-ethanol;
(R)-2-{1,1-dimethyl-3-[(1s,4R)-4-hydroxycyclohexyl]propylamino}-1-(5-fluoro-3-pyridyl)-1-ethanol;
(R)-2-{1,1-dimethyl-2-[(1r,4R)-4-hydroxycyclohexyl]ethylamino}-1-(m-fluorophenyl)-1-ethanol;
(R)-2-{1,1-dimethyl-2-[(1s,4S)-4-hydroxycyclohexyl]ethylamino}-1-(5-fluoro-3-pyridyl)-1-ethanol;
N-[(1R,4r)-4-{[(R)-2-(m-fluorophenyl)-2-hydroxyethylamino]methyl}cyclohexyl]benzamide;
(R)-2-{1,1-dimethyl-3-[(1r,4S)-4-hydroxycyclohexyl]propylamino}-1-(o-fluorophenyl)-1-ethanol;
(R)-2-{1,1-dimethyl-3-[(1s,4R)-4-hydroxycyclohexyl]propylamino}-1-(o-fluorophenyl)-1-ethanol;
methyl (1S,4s)-4-{2-[(R)-2-(5-fluoro-3-pyridyl)-2-hydroxyethylamino]-2-methylpropyl}cyclohexanecarboxylate;
(S)-[(2R,5S)-5-{[(1r,4S)-4-methoxycyclohexyl]methyl}-2-pyrrolidinyl](m-fluorophenyl)methanol;
(R)-[(2R,5S)-5-{[(1r,4S)-4-methoxycyclohexyl]methyl}-2-pyrrolidinyl](m-fluorophenyl)methanol;
methyl (1S,4s)-4-{2-[(R)-2-(m-fluorophenyl)-2-hydroxyethylamino]-2-methylpropyl}cyclohexanecarboxylate;
(1S,4s)-8-[(R)-2-(m-fluorophenyl)-2-hydroxyethylamino]-7-methoxy-p-menthan-7-one;
(R)-(m-fluorophenyl){4-(methoxymethyl)-7-azabicyclo[2.2.1]hept-1-yl}methanol;
(R)-[(2R,5R)-5-{[(1r,4R)-4-methoxycyclohexyl]methyl}-2-pyrrolidinyl](m-fluorophenyl)methanol;
(R)-[(2R,5S)-5-{[(1r,4S)-4-methoxycyclohexyl]methyl}-2-pyrrolidinyl](o-fluorophenyl)methanol;
(R)-[(2R,5R)-5-{[(1r,4R)-4-methoxycyclohexyl]methyl}-2-pyrrolidinyl](o-fluorophenyl)methanol;
(R)-[(2R,5R)-5-{[(1r,4R)-4-methoxycyclohexyl]methyl}-2-pyrrolidinyl](5-fluoro-3-pyridyl)methanol;
methyl (1R,4r)-4-{3-[(R)-2-(5-fluoro-3-pyridyl)-2-hydroxyethylamino]-3-methylbutyl}cyclohexanecarboxylate;
N-[(1R,4r)-4-{1-[(R)-2-(5-fluoro-3-pyridyl)-2-hydroxyethylamino]-1-methylethyl}cyclohexyl]acetamide;
(1S,4s)-4-{3-[(R)-2-(5-fluoro-3-pyridyl)-2-hydroxyethylamino]-3-methylbutyl}cyclohexanecarboxylic acid;
methyl (1S,4s)-4-{3-[(R)-2-(5-fluoro-3-pyridyl)-2-hydroxyethylamino]-3-methylbutyl}cyclohexanecarboxylate;
methyl (1R,4r)-4-{3-[(R)-2-(m-fluorophenyl)-2-hydroxyethylamino]-3-methylbutyl}cyclohexanecarboxylate;
methyl (1S,4s)-4-{3-[(R)-2-(m-fluorophenyl)-2-hydroxyethylamino]-3-methylbutyl}cyclohexanecarboxylate;
methyl (1R,4r)-4-{2-[(R)-2-(m-fluorophenyl)-2-hydroxyethylamino]-2-methylpropyl}cyclohexanecarboxylate;
N-[(1R,4r)-4-{2-[(R)-2-(5-fluoro-3-pyridyl)-2-hydroxyethylamino]-2-methylpropyl}cyclohexyl]acetamide;
(R)-2-{1,1-dimethyl-2-[(1r,4R)-4-aminocyclohexyl]ethylamino}-1-(5-fluoro-3-pyridyl)-1-ethanol;
(R)-2-{1,1-dimethyl-2-[(1r,4R)-4-(mesylamino)cyclohexyl]ethylamino}-1-(5-fluoro-3-pyridyl)-1-ethanol;
N-[(1R,4r)-4-{3-[(R)-2-(5-fluoro-3-pyridyl)-2-hydroxyethylamino]-3-methylbutyl}cyclohexyl]acetamide;
(S)-[(2R,5S)-5-methyl-5-{[(1r,4S)-4-methoxycyclohexyl]methyl}-2-pyrrolidinyl](m-fluorophenyl)methanol;
(S)-{(2R,5R)-5-[(p-methoxyphenyl)methyl]-5-methyl-2-pyrrolidinyl}(m-fluorophenyl)methanol;
(R)-{(2R,5R)-5-[(p-methoxyphenyl)methyl]-5-methyl-2-pyrrolidinyl}(m-fluorophenyl)methanol;
(1R,4r)-8-[(R)-2-(m-fluorophenyl)-2-hydroxyethylamino]-7-methoxy-p-menthan-7-one;
(1R,4r)-8-[(R)-2-(5-fluoro-3-pyridyl)-2-hydroxyethylamino]-7-methoxy-p-menthan-7-one;
(R)-2-{1,1-dimethyl-2-[(1r,4R)-4-aminocyclohexyl]ethylamino}-1-(m-fluorophenyl)-1-ethanol;
(S)-{(2R,5S)-5-[(p-methoxyphenyl)methyl]-2-pyrrolidinyl}(m-fluorophenyl)methanol;
(R)-{(2R,5S)-5-[(p-methoxyphenyl)methyl]-2-pyrrolidinyl}(m-fluorophenyl)methanol;
(R)-{(2R,5R)-5-[(p-methoxyphenyl)methyl]-2-pyrrolidinyl}(m-fluorophenyl)methanol;
(S)-{(2R,5R)-5-[(p-methoxyphenyl)methyl]-2-pyrrolidinyl}(m-fluorophenyl)methanol;
(R)-2-(tert-butylamino)-1-(2-methyl-3-pyridyl)-1-ethanol;
(S)-{(2R,5S)-5-[(p-methoxyphenyl)methyl]-2-pyrrolidinyl}(5-fluoro-3-pyridyl)methanol;
(R)-{(2R,5S)-5-[(p-methoxyphenyl)methyl]-2-pyrrolidinyl}(5-fluoro-3-pyridyl)methanol;
(R)-{(2R,5R)-5-[(p-methoxyphenyl)methyl]-2-pyrrolidinyl}(5-fluoro-3-pyridyl)methanol;
(S)-{(2R,5R)-5-[(p-methoxyphenyl)methyl]-2-pyrrolidinyl}(5-fluoro-3-pyridyl)methanol;
(R)-2-{1,1-dimethyl-2-[(1r,4R)-4-(mesylamino)cyclohexyl]ethylamino}-1-(m-fluorophenyl)-1-ethanol;
N-[(1R,4r)-4-{2-[(R)-2-(m-fluorophenyl)-2-hydroxyethylamino]-2-methylpropyl}cyclohexyl]acetamide;
N-[(1R,4r)-4-{1-[(R)-2-(m-fluorophenyl)-2-hydroxyethylamino]-1-methylethyl}cyclohexyl]acetamide;
6-[(S)-2-(tert-butylamino)-1-hydroxyethyl]-2-pyridinecarbonitrile;
(R)-(4-benzyl-7-azabicyclo[2.2.1]hept-1-yl)(m-fluorophenyl)methanol;
(R)-{(2R,5S)-5-[(p-chlorophenyl)methyl]-2-pyrrolidinyl}(m-fluorophenyl)methanol;
(R)-{(2R,5R)-5-[(p-chlorophenyl)methyl]-2-pyrrolidinyl}(m-fluorophenyl)methanol;
(R)-(4-{[(p-chlorophenyl)methoxy]methyl}-7-azabicyclo[2.2.1]hept-1-yl)(m-fluorophenyl)methanol;
(S)-2-(tert-butylamino)-1-[6-(trifluoromethyl)-2-pyridyl]-1-ethanol;
ethyl [(1R,4r)-4-{1-[(R)-2-(m-fluorophenyl)-2-hydroxyethylamino]-1-methylethyl}cyclohexyloxy]acetate;
(1S,4s)-4-{2-[(R)-2-(m-fluorophenyl)-2-hydroxyethylamino]-2-methylpropyl}cyclohexanecarbonitrile;
(R)-(m-fluorophenyl){4-[(p-methoxyphenyl)methyl]-7-azabicyclo[2.2.1]hept-1-yl}methanol;
(R)-{(2R,5S)-5-[(p-chlorophenyl)methyl]-2-pyrrolidinyl}(5-fluoro-3-pyridyl)methanol;
(S)-{(2R,5R)-5-[(p-chlorophenyl)methyl]-2-pyrrolidinyl}(5-fluoro-3-pyridyl)methanol;
(R)-{(2R,5R)-5-[(p-chlorophenyl)methyl]-2-pyrrolidinyl}(5-fluoro-3-pyridyl)methanol;
ethyl [(1R,4r)-4-{1-[(R)-2-(5-fluoro-3-pyridyl)-2-hydroxyethylamino]-1-methylethyl}cyclohexyloxy]acetate;
(R)-(m-fluorophenyl){4-[(p-fluorophenyl)methyl]-7-azabicyclo[2.2.1]hept-1-yl}methanol;
6-[(S)-2-{1,1-dimethyl-2-[(1r,4S)-4-aminocyclohexyl]ethylamino}-1-hydroxyethyl]-2-pyridinecarbonitrile;
N-[(1R,4s)-4-{2-[(S)-2-(6-cyano-2-pyridyl)-2-hydroxyethylamino]-2-methylpropyl}cyclohexyl]acetamide;
N-[(1S,4r)-4-{2-[(S)-2-(6-cyano-2-pyridyl)-2-hydroxyethylamino]-2-methylpropyl}cyclohexyl]acetamide;
6-[(S)-2-{1,1-dimethyl-2-[(1r,4S)-4-(mesylamino)cyclohexyl]ethylamino}-1-hydroxyethyl]-2-pyridinecarbonitrile;
N-[(1R,4r)-4-{2-[(R)-2-(m-fluorophenyl)-2-hydroxyethylamino]-2-methylpropyl}cyclohexyl]2,2-dimethylpropionamide;
N-[(1R,4r)-4-{2-[(R)-2-(m-fluorophenyl)-2-hydroxyethylamino]-2-methylpropyl}cyclohexyl]trifluoroacetamide;
N-[(1R,4r)-4-{2-[(R)-2-hydroxy-2-(2-methyl-3-pyridyl)ethylamino]-2-methylpropyl}cyclohexyl]acetamide;
(R)-(m-fluorophenyl)(4-{[(p-fluorophenyl)methoxy]methyl}-7-azabicyclo[2.2.1]hept-1-yl)methanol;
(S)-{4-[(p-chlorophenoxy)methyl]-7-azabicyclo[2.2.1]hept-1-yl}(m-fluorophenyl)methanol;
(R)-{4-[(p-chlorophenoxy)methyl]-7-azabicyclo[2.2.1]hept-1-yl}(m-fluorophenyl)methanol;
(R)-(4-{[(p-fluorophenyl)methoxy]methyl}-7-azabicyclo[2.2.1]hept-1-yl)(5-fluoro-3-pyridyl)methanol;
(S)-(m-fluorophenyl){4-[2-(3-pyridyl)ethyl]-7-azabicyclo[2.2.1]hept-1-yl}methanol;
N-[(1S,4s)-4-{2-[(R)-2-(m-fluorophenyl)-2-hydroxyethylamino]-2-methylpropyl}cyclohexyl]cyclopropanecarboxamide;
N-[(1S,4s)-4-{2-[(R)-2-(m-fluorophenyl)-2-hydroxyethylamino]-2-methylpropyl}cyclohexyl]acetamide;
(R)-2-{1,1-dimethyl-2-[(1s,4S)-4-(mesylamino)cyclohexyl]ethylamino}-1-(m-fluorophenyl)-1-ethanol;
N-[(1S,4s)-4-{2-[(R)-2-(m-fluorophenyl)-2-hydroxyethylamino]-2-methylpropyl}cyclohexyl]trifluoroacetamide;
(R)-2-{1,1-dimethyl-2-[(1s,4S)-4-aminocyclohexyl]ethylamino}-1-(m-fluorophenyl)-1-ethanol;
(R)-2-{1,1-dimethyl-2-[(1s,4S)-4-(3,3-dimethylureido)cyclohexyl]ethylamino}-1-(m-fluorophenyl)-1-ethanol;
(R)-2-{1,1-dimethyl-2-[(1r,4R)-4-(trifluoromesylamino)cyclohexyl]ethylamino}-1-(m-fluorophenyl)-1-ethanol;
(1S,4r)-4-(2-{(S)-2-hydroxy-2-[6-(trifluoromethyl)-2-pyridyl]ethylamino}-2-methylpropyl)cyclohexyl 2-methyl-2-propanecarbamate;
N-[(1S,4s)-4-{2-[(R)-2-(5-fluoro-3-pyridyl)-2-hydroxyethylamino]-2-methylpropyl}cyclohexyl]acetamide;
(R)-2-{1,1-dimethyl-2-[(1s,4S)-4-(mesylamino)cyclohexyl]ethylamino}-1-(5-fluoro-3-pyridyl)-1-ethanol;
(1S,4s)-4-{2-[(R)-2-(5-fluoro-3-pyridyl)-2-hydroxyethylamino]-2-methylpropyl}cyclohexyl 2-methyl-2-propanecarbamate;
(R)-2-{1,1-dimethyl-2-[(1s,4S)-4-(dimethylaminosulfonyl)cyclohexyl]ethylamino}-1-(m-fluorophenyl)-1-ethanol;
(R)-2-{1,1-dimethyl-2-[(1r,4R)-4-(methylaminosulfonyl)cyclohexyl]ethylamino}-1-(m-fluorophenyl)-1-ethanol;
N-[(1R,4r)-4-{2-[(R)-2-(m-fluorophenyl)-2-hydroxyethylamino]-2-methylpropyl}cyclohexyl]-N-methylacetamide;
N-[(1R,4r)-4-{2-[(R)-2-(5-fluoro-3-pyridyl)-2-hydroxyethylamino]-2-methylpropyl}cyclohexyl]-N-methylacetamide;
(R)-[(2R,5S)-5-{[(1s,4R)-4-(mesylamino)cyclohexyl]methyl}-2-pyrrolidinyl](m-fluorophenyl)methanol;
(1R,4r)-4-{2-[(R)-2-(m-fluorophenyl)-2-hydroxyethylamino]-2-methylpropyl}cyclohexanesulfonamide;
(1S,4s)-4-{2-[(R)-2-(5-fluoro-3-pyridyl)-2-hydroxyethylamino]-2-methylpropyl}cyclohexanesulfonamide;
(R)-2-{1,1-dimethyl-2-[(1s,4S)-4-(trifluoromesylamino)cyclohexyl]ethylamino}-1-(5-fluoro-3-pyridyl)-1-ethanol;
(R)-{4-[(E)-3-phenyl-2-propenyl]-7-azabicyclo[2.2.1]hept-1-yl}(5-fluoro-3-pyridyl)methanol;
(R)-{4-[(E)-3-phenyl-2-propenyl]-7-azabicyclo[2.2.1]hept-1-yl}(m-fluorophenyl)methanol;
(R)-(m-fluorophenyl){4-(3-phenylpropyl)-7-azabicyclo[2.2.1]hept-1-yl}methanol; and
(S)-(o-fluorophenyl){4-[2-(3-pyridyl)ethyl]-7-azabicyclo[2.2.1]hept-1-yl}methanol,
   and pharmaceutically acceptable salts thereof.

As described herein, it will also be understood that certain compounds acting as β₂-adrenergic receptor agonists are able to activate the β₂-adrenergic receptor without significant recruitment of p-arrestin.

Thus, particular β₂-adrenergic receptor agonists that may be mentioned include those able to activate the β₂-adrenergic receptor without significant recruitment of beta-arrestin.

Further, in particular embodiments, the methods and uses as described herein may be performed without significant recruitment of p-arrestin.

The skilled person will be able to determine the level of recruitment of p-arrestin provided by compounds, such as those referred to herein, using techniques known to those skilled in the art, such as those described in the examples as provided herein.

### Methods of Treatment

The skilled person will understand that compounds and pharmaceutical formulations for use, and methods of the invention, as defined herein are useful the treatment or prophylaxis of a disease or disorder characterised by muscle wasting.

The skilled person will understand that a disease or disorder characterised by muscle wasting will refer to a disease or disorder of which muscle wasting (which will include muscular atrophy, increased muscle weakness/reduced muscle strength and/or reduced muscle mass) is a significant clinical manifestation.

As described herein, muscular atrophy and weakness are pathological manifestations of various neurological, neuromuscular and myogenic disorders. These can also present themselves as secondary symptoms in conditions such as cancer, cardiopulmonary disorders and are also hallmarks of progressive aging.

As such, compounds and pharmaceutical formulations for use, and methods of the invention, as defined herein may be useful in treating or preventing muscle wasting by reducing or preventing (e.g. reducing, such as reducing to a clinically significant extent) degeneration of muscular tissue.

In particular embodiments, the disease or disorder characterised by muscle wasting is a neurological, neuromuscular or myogenic disorder.

In alternative embodiments, the muscle wasting is a secondary symptom, such as occurring in conditions such as cancer and cardiopulmonary disorders.

In alternative embodiments, the muscle wasting is a secondary symptom of progressive aging.

In other embodiments, the muscle wasting is not primarily induced by (i.e. has a primary cause other than) progressive aging.

In particular embodiments, is the disease or disorder characterised by muscle wasting is resulting from a generic abnormality, which may be referred to as a hereditary disease or disorder.

In alternative embodiments, is the disease or disorder characterised by muscle wasting is non-genetic (i.e. non-hereditary).

In particular embodiments, is the disease or disorder characterised by muscle wasting is muscular dystrophy.

For the avoidance of doubt, the term “muscular dystrophy” will take its normal meaning in the art, e.g. referring to a group of neuromuscular diseases that cause progressive weakness and loss of muscle mass; in particular, in which genetic abnormalities inhibit the production of proteins needed to form healthy muscle tissue.

As such, muscular dystrophy will be understood by those skilled in the art to refer to a group of genetic disorders characterized by progressive muscle degeneration. In particular, such conditions may be identified based on the alterations of the protein called dystrophin, leading to degeneration of muscular tissue.

As such, compounds and pharmaceutical formulations for use, and methods of the invention, as defined herein may be useful in treating or preventing muscular dystrophy by reducing or preventing (e.g. reducing, such as reducing to a clinically significant extent) degeneration of muscular tissue.

The skilled person will understand that the class of disorders referred to as muscular dystrophy may also be known to those skilled in the art as dystrophinopathies. This class of disorders will include:
Spinal muscular atrophy (SMA) I;
SMA II;
SMA III;
Spinal and bulbar muscular atrophy (SBMA);
Facioscapulohumeral muscular dystrophy (FSHD);
Myasthenia Gravis;
Atrophy due to disuse;
Post-surgical muscle recovery;
Muscle wasting due to cachexia;
Age related Sarcopenia MS;
Acid maltase Deficiency;
ALS;
Central core disease;
Muscular Atrophy arising due to pharmacological treatment, e.g. dexamethasone;
Duchenne muscular dystrophy (DMD);
Becker muscular dystrophy (BMD, a mild form of DMD);
an intermediate clinical presentation between DMD and BMD; and
DMD-associated dilated cardiomyopathy (heart-disease), which may be associated with little or no clinical skeletal, or voluntary, muscle disease.

In a particular embodiment, the muscular dystrophy is selected from the group consisting of:
Spinal muscular atrophy (SMA) I;
SMA II;
SMA III;
Duchenne muscular dystrophy (DMD); and
Becker muscular dystrophy (BMD).

In a more particular embodiment, the muscular dystrophy is Duchenne muscular dystrophy (DMD).

In a particular embodiment, the disease or disorder characterised by muscle wasting is sarcopenia (such as age-related sarcopenia).

Sarcopenia is a condition characterized by loss of skeletal muscle mass and function.

Sarcopenia is recognized as a disease, and it has been recently assigned an ICD-10-CM (M62.84). An example of a screening test for sarcopenia is SARC-F.

The loss in muscle mass may be associated with increased body fat so that despite normal weight there is marked weakness. This condition is known as sarcopenic obesity.

In more particular embodiments, the age-related sarcopenia is sarcopenic obesity.

Severe skeletal muscle wasting may occur during hospitalisation, in particular during intensive care unit treatment. The generalized muscle weakness that develops in both limb and respiratory muscles during the course of ICU hospitalization has been termed “intensive care unit acquired weakness” (ICUAW).

In particular embodiments, the disease or disorder characterised by muscle wasting is intensive care unit acquired weakness (ICUAW).

Cancer cachexia is a complex metabolic syndrome relates to underlying illness and characterised by muscle mass loss, with or without fat loss. A common underlying illness is cancer.

In particular embodiments, the disease or disorder characterised by muscle wasting is cachexia, such as cancer cachexia.

### Weight Loss Treatment

In particular embodiments, the treatment and/or prophylaxis of muscle wasting is treatment and/or prophylaxis of muscular atrophy in a patient undergoing weight loss.

The skilled person will understand that references to a patient undergoing weight loss treatment will refer to a patient who is undergoing a loss of weight.

The skilled person will understand that the term “undergoing” will take its normal meaning, including by referring to the patient being subject to and/or in the process of said event at the relevant time (i.e. on a continuing basis). As such, the term “undergoing” may be replaced with terms such as “experiencing”, “undertaking”, and the like.

Depending on the nature of the weight loss, the patient undergoing such may be doing so as a result of the advice/actions of a clinician or other health practitioner (i.e. as a result of a therapeutic intervention) or at their own instigation (i.e. unsupervised).

In particular embodiments, the weight loss will be a result of one or more therapeutic intervention.

In a more particular embodiment, the weight loss will be a result of one or more therapeutic intervention with the effect of inducing loss of body weight, which will refer in particular to a lowering of body fat composition.

In particular embodiments, the therapeutic intervention will be a long-term treatment, which may refer to a treatment period of greater than one month (e.g. greater than 3 months, greater than six months or greater than one year).

For the avoidance of doubt, the therapeutic intervention (e.g. treatment with one or more therapeutic agent, i.e. pharmaceutical) will include those having weight loss as a primary purpose (i.e. those labelled and/or marketed for the purposes of achieving such an effect) and those having weight loss as a secondary effect (i.e. those labelled as having weight loss as an effect that is not the primary purpose, e.g. as a potential adverse event/side effect associated with the therapeutic intervention, such as therapeutic agent).

In more particular embodiments, the therapeutic intervention will have weight loss as a primary purpose, which may be referred to as weight loss treatment.

Thus, in particular embodiments, the weight loss will be referred to as weight loss treatment.

The skilled person will understand that weight loss treatments, such as those described herein, may be therapeutic (i.e. in a patient in medical need of such treatment, such as in an obese patient) or non-therapeutic (e.g. cosmetic).

For example, weight loss treatments may refer to those in which the weight loss is induced through reduction of caloric intake and/or increased energy expenditure (i.e. increased metabolism; such as through exercise), which may be voluntary (i.e. resulting from the actions of the patient) or induced (i.e. the result of an intervention, such as a therapeutic intervention conducted by a clinician or other health practitioner).

In certain embodiments, weight loss treatments described herein will be therapeutic.

In certain embodiments, weight loss treatments described herein will be non-therapeutic (e.g. cosmetic).

As described herein, weight loss treatments will include those that are non-medical, such as those comprising modified diets (e.g. based on reduced caloric intake).

Particular weight loss treatments that may be mentioned are medical weight loss treatments, which will include surgical methods and treatments with therapeutic agents (i.e. pharmaceutical treatments).

For example, surgical weight loss treatments that may be mentioned include those involving reduction of stomach capacity, such as through the fitting of a gastric band.

Particular weight loss treatments that may be mentioned include those in which the weight loss is induced through reduction of caloric intake and/or increase in metabolism (such that the patient will experience overall caloric deficiency).

In a particular embodiment, the weight loss treatment is treatment with a therapeutic (i.e. pharmaceutical) agent, which may be referred to herein as a therapeutic weight loss agent.

In particular embodiments, weight loss treatment (e.g. treatment with a therapeutic weight loss agent) will refer to:
(i) the treatment or prophylaxis of obesity; and/or
(ii) lowering body fat composition and/or reducing body weight.

In more particular embodiments, such weight loss treatment will refer to the treatment or prophylaxis of obesity.

In yet more particular embodiments, such weight loss treatment will refer to the treatment or prophylaxis of chronic obesity.

In alternative embodiments, such weight loss treatment will refer to lowering body fat composition and/or reducing body weight.

For the avoidance of doubt, the term “obesity” as used herein will be understood by those skilled in the art to refer to a condition characterised by abnormal or excessive fat accumulation that may impair health, which conditions will be readily identified by those skilled in the art.

For example, in particular embodiments obesity may be understood to be a condition characterised by abnormal or excessive fat accumulation that may impair health in which the subject (e.g. an adult subject) has a body mass index (BMI) of 30.0 or higher (e.g. 30.0 to 39.9).

The skilled person will understand that the term “chronic” will take its normal meaning 5 in the art, such as persisting for an extended time (e.g. such as a period of greater than one month, such as greater than three months, greater than six months or greater than one year).

For the avoidance of doubt, references to obesity will include references to conditions referred to as “morbid” and/or “clinical” obesity.

For the avoidance of doubt, references to the “lowering of body fat composition” will refer to the lowering of the amount of body fat in the patient, which may be indicated by a reduction in body fat percentage.

In particular, references to the lowering of body fat composition will refer to reducing levels of body fat in the form of adipose tissue. Similarly, references to the treatment or prophylaxis of obesity may refer to the treatment or prophylaxis of obesity by reducing levels of body fat in the form of adipose tissue.

In particular, references to the treatment or prophylaxis of obesity may include references to the treatment or prophylaxis of obesity by lowering body fat composition (e.g. by reducing levels of body fat in the form of adipose tissue) and/or reducing body weight.

For example, references to the treatment or prophylaxis of obesity may include references to the treatment or prophylaxis of obesity by lowering body fat composition (e.g. by reducing levels of body fat in the form of adipose tissue).

In particular embodiments, the patient is obese.

In particular embodiments, references to lowering body fat composition and/or reducing body weight may refer to therapeutic methods and uses in the lowering body fat composition and/or reducing body weight, such as uses and methods performed in an obese patient.

In particular embodiments, references to lowering body fat composition and/or reducing body weight may refer to lowering body fat composition and/or reducing body weight in a patient (or subject) in need thereof, e.g. a patient who has an above-normal body weight or BMI (e.g. a BMI of 30 or greater, i.e. an obese patient), which may be referred to as therapeutically lowering body fat composition and/or reducing body weight.

Alternatively, references to lowering body fat composition and/or reducing body weight may refer to in lowering body fat composition and/or reducing body weight in a patient (or subject) who has a normal body weight or who is overweight (in each case, with a corresponding BMI). In particular instances, such subjects (e.g. adult subjects) will be non-obese (e.g. having a BMI of less than 30.0), e.g. subjects being overweight (BMI 25.0 to 29.9) or of a healthy weight (BMI 18.5 to 24.9), which may be referred to as non-therapeutically lowering body fat composition and/or reducing body weight. As such, the skilled person will understand that such uses may be performed in patients who are not defined as being obese (e.g. in patients who are defined as being of a healthy weight or overweight but not obese).

In particular embodiments, the patient (or subject) is overweight.

For the avoidance of doubt, references to non-therapeutic uses and methods will refer to uses and methods in patients that are not directed to the treatment of a medical condition but which provide the relevant effects for other purposes, such as for cosmetic purposes.

As described herein, other uses in medical treatment as described herein may be understood to be further characterised by lowering body fat composition and/or reducing body weight. For example, the treatment or prophylaxis of obesity as described herein may be achieved by lowering body fat composition and/or reducing body weight (e.g. lowering body fat composition).

For the avoidance of doubt, references herein to body fat may refer in particular to body fat in the form of adipose tissue.

As described herein, the weight loss treatment may comprise (or will consist of) weight loss treatment with a therapeutic agent (i.e. a pharmaceutical), which may be referred to as a therapeutic weight loss agent.

For the avoidance of doubt, therapeutic weight loss agents (including therapeutic agents for the treatment or prophylaxis of obesity, and/or lowering body fat composition and/or reducing body weight) will be known to those skilled in the art and will include those having weight loss as a primary purpose (i.e. those labelled and/or marketed for the purposes of achieving such an effect).

Thus, in particular embodiments, the term “therapeutic weight loss agents” will refer to therapeutic agents having weight loss as a primary purpose.

In particular embodiments, the weight loss treatment will comprise (or will consist of) treatment (i.e. weight loss treatment) with one or more (e.g. one) therapeutic weight loss agent for:
(i) the treatment or prophylaxis of obesity; and/or
(ii) lowering body fat composition and/or reducing body weight.

In more particular embodiments, the weight loss treatment will comprise (or will consist of) treatment with one or more therapeutic weight loss agent for the treatment or prophylaxis of obesity.

In more particular embodiments, the weight loss treatment will comprise (or will consist of) treatment with one or more therapeutic weight loss agent for the treatment or prophylaxis of chronic obesity.

In alternative embodiments, the weight loss treatment will comprise treatment with one or more therapeutic weight loss agent for lowering body fat composition and/or reducing body weight.

For the avoidance of doubt, the therapeutic agent may be referred to as a therapeutic weight loss agent.

In particular, therapeutic weight loss agents for the treatment or prophylaxis of obesity and/or lowering body fat composition and/or reducing body weight will include those acting by inducing a reduction in caloric intake and/or an increase in metabolism.

Particular therapeutic weight loss agents (e.g. therapeutic agents for the treatment or prophylaxis of obesity) that may be mentioned include GLP-1 receptor agonists.

Thus, in particular embodiments, the therapeutic weight loss agent (i.e. the weight loss treatment) is a GLP-1 receptor agonist.

In particular embodiments, the therapeutic weight loss agent (i.e. the weight loss treatment) is a GLP-1 receptor agonist.

Therefore, in particular embodiments, the patient undergoing weight loss is undergoing weight loss as a consequence of (i.e. a result of/an effect of) treatment with a GLP-1 receptor agonist.

Thus, for the avoidance of doubt, within the scope of the present invention is a β₂-adrenergic receptor agonist, or a pharmaceutically acceptable salt thereof, for use in the treatment or prophylaxis of muscular atrophy in a patient also being treated (or undergoing treatment) with a therapeutic weight loss agent (such as a GLP-1 receptor agonist).

Also within the scope of the present invention is a method for the treatment or prophylaxis of muscular atrophy in a patient also being treated (or undergoing treatment) with a therapeutic weight loss agent (such as a GLP-1 receptor agonist) comprising administration of a therapeutically effective amount of a β₂-adrenergic receptor agonist, or a pharmaceutically acceptable salt thereof, to a patient in need thereof.

Various GLP-1 receptor agonists will be known to those skilled in the art and will include those currently authorised for marketing in the US (i.e. by the Food and Drug Administration) and Europe (e.g. by the European Medicines Agency).

Particular GLP-1 receptor agonists that may be mentioned include exenatide, exenatide extended-release, tirzepatide, liraglutide, lixisenatide, semaglutide, albiglutide and dulaglutide.

In particular embodiments, the GLP-1 receptor agonist is liraglutide or semaglutide.

In more particular embodiments, the GLP-1 receptor agonist is liraglutide.

The skilled person will understand that GLP-1 receptor agonists, i.e. agonists of the Glucagon-like peptide 1 receptor, as well as derivatives thereof, mimic the action of Glucagon-like peptide 1.

The GLP-1 receptor agonist may have affinity to the GIP receptor, i.e. the Gastric Inhibitory Polypeptide receptor, in addition to the GLP-1 receptor. Alternatively, the GLP-1 receptor agonist may be free or substantially free from affinity to the GIP receptor.

The skilled person will understand that GLP-1 receptor agonist may be a peptide. Further, it may be provided as a liquid or suspension, in injectable form such as in a prefilled pen for injection and/or as an extended release formulation. In an example, the GLP-1 receptor agonist may administrated subcutaneously. The GLP-1 receptor agonist may be administered once or several times daily or weekly.

The following are examples of GLP-1 receptor agonists.

Exenatide, which has the CAS number 141758-74-9, is a peptide sold under the trade names Byetta and Bydureon. It may be administered subcutaneously and/or in a dosage from 5 micrograms to 2 mg.

Tirzepatide, which has the CAS number 2023788-19-2, is a linear polypeptide of 39 amino acids that has been chemically modified by lipidation. It is sold under the trade name Mounjaro. It may be administered subcutaneously as an injectable solution. The dosage may be from 2.5 mg/0.5 mL to 15 mg/0.5 mL.

Liraglutide, which has the CAS number 204656-20-2, is a peptide sold under the brand names Saxenda, Victoza and Xultophy. It may be administered subcutaneously as an injectable solution. The dosage may be from 0.6 mg to 1.8 mg such as once daily for a week or more. For example, the dosage may be 0.6 mg, 1.2 mg or 1.8 such as once daily for a week or more.

Lixisenatide, which has the CAS number 320367-13-3, sold under the brand names Lyxumia and Adlyxin. It may be administrated in injectable form such as subcutaneously. For example, it may be administrated once daily. The dosage may be from 10 micrograms to 20 micrograms such as once weekly.

Semaglutide, which has the CAS number 910463-68-2, is a peptide sold under the brand names Ozempic, Rybelsus and Wegovy. It may be administrated in injectable form, such as subcutaneously (Ozempic and Wegovy), or orally (Rybelsus). The dosage may be from 0.25 mg to 2 mg, administered weekly.

Albiglutide, which has the CAS number 782500-75-8, is a peptide sold under the trade names Eperzan and Tanzeum. It may also be referred to as GSK-716155. It may be administrated in injectable form such as subcutaneously. The dosage may be from 30 mg to 50 mg such as once weekly.

Dulaglutide, which has the CAS number 923950-08-7, is a peptide sold under the brand name Trulicity among others. It may be administered in a pen for injection. For instance, it may be administered subcutaneously. The dosage of Dulaglutide may be 0.75 mg/0.5 mL, 1.5 mg/0.5 mL, 3 mg/0.5 mL or 4.5 mg/0.5 mL.

### Pharmaceutical Formulations

The skilled person will understand that both the β₂-adrenergic receptor agonist, or pharmaceutically acceptable salt thereof, and optionally the therapeutic weight loss agent, as described herein, may be administered in the form of a pharmaceutical formulation, which may optionally further comprise one or more pharmaceutically acceptable excipient.

Suitable pharmaceutical formulations may be commercially available or otherwise are described in the literature, such as, Remington, The Science and Practice of Pharmacy, 19th ed., Mack Printing Company, Easton, Pennsylvania (1995), and Martindale—The Complete Drug Reference (35^{th} Edition), and the documents referred to therein, the relevant disclosures in all of which documents are hereby incorporated by reference in their entirety. Otherwise, the preparation of suitable formulations, and in particular combined preparations including both a β₂-adrenergic receptor agonist, or pharmaceutically acceptable salts thereof, and optionally the therapeutic weight loss agent may be achieved by the skilled person using routine techniques.

References to pharmaceutically acceptable excipient(s) may be understood to include pharmaceutically acceptable, diluents, carriers and/or adjuvants, as known to those skilled in the art.

In a second aspect of the invention, there is provided a pharmaceutical formulation comprising a β₂-adrenergic receptor agonist, or a pharmaceutically acceptable salt thereof, and optionally one or more pharmaceutically acceptable excipient, for use in the treatment or prophylaxis of a disease or disorder characterised by muscle wasting.

In an alternative second aspect of the invention, there is provided a method for the treatment or prophylaxis of a disease or disorder characterised by muscle wasting, comprising administering to a patient in need thereof a therapeutically effective amount of a pharmaceutical formulation comprising a β₂-adrenergic receptor agonist, or a pharmaceutically acceptable salt thereof, and optionally one or more pharmaceutically acceptable excipient.

For the avoidance of doubt, the formulations for use of the second aspect of the invention may have any of the particular features described above for the first aspect of the invention, including all combinations thereof.

In a third aspect of the invention, there is provided a pharmaceutical formulation comprising:
(a) a β₂-adrenergic receptor agonist, or a pharmaceutically acceptable salt thereof; and
(b) a therapeutic weight loss agent,
   and optionally one or more pharmaceutically acceptable excipient.

In a particular embodiment, the pharmaceutical formulation is for use in the treatment or prophylaxis of a disease or disorder.

In a more particular embodiment, the pharmaceutical formulation is for use in the treatment or prophylaxis of muscular atrophy in a patient undergoing weight loss treatment, as defined herein.

Thus in a fourth aspect of the invention, there is provided a pharmaceutical formulation comprising:
(a) a β₂-adrenergic receptor agonist, or a pharmaceutically acceptable salt thereof; and
(b) a therapeutic weight loss agent,
   and optionally one or more pharmaceutically acceptable excipient, for use in the treatment or prophylaxis of muscular atrophy in a patient undergoing weight loss treatment.

In an alternative fourth aspect of the invention, there is provided a method for the treatment or prophylaxis of muscular atrophy in a patient undergoing weight loss treatment, comprising administering to a patient in need thereof a therapeutically effective amount of a pharmaceutical formulation comprising:
(a) a β₂-adrenergic receptor agonist, or a pharmaceutically acceptable salt thereof; and
(b) a therapeutic weight loss agent,
   and optionally one or more pharmaceutically acceptable excipient.

For the avoidance of doubt, the formulations of the second, third and fourth aspects of the invention may have any of the particular features described above for the first aspect of the invention, including all combinations thereof.

Thus, in particular embodiments of the third and fourth aspects of the invention the therapeutic weight loss agent is as described in the first aspect of the invention (including all embodiments and features thereof).

### Combinations and Kits-of-Parts (Weight Loss)

The skilled person will understand that the present invention includes a combination (i.e. a combination product) of a β₂-adrenergic receptor agonist, or a pharmaceutically acceptable salt thereof, and a therapeutic weight loss agent, which may also be provided in the form of a kit-of-parts comprising the same.

In a fifth aspect of the invention, there is provided a combination or kit-of-parts comprising components:
(A) a pharmaceutical formulation comprising a β₂-adrenergic receptor agonist, or a pharmaceutically acceptable salt thereof, optionally in admixture with one or more pharmaceutically acceptable excipient, and
(B) a pharmaceutical formulation comprising a therapeutic weight loss agent, optionally in admixture with one or more pharmaceutically acceptable excipient,
   wherein components (A) and (B) are each provided in a form that is suitable for administration in conjunction with the other.

In a particular embodiment, the combination or kit-of-parts is for use in the treatment or prophylaxis of muscular atrophy in a patient undergoing weight loss.

In a particular embodiment, there is provided a method for the treatment or prophylaxis of muscular atrophy in patient undergoing weight loss comprising administering to a patient in need thereof a therapeutically effective amount of the combination or kit-of-parts.

In a particular embodiment, the kit-of-parts of the fifth aspect of the invention further comprises instructions to use each component in conjunction with the other in the treatment or prophylaxis of muscular atrophy in a patient undergoing weight loss treatment.

For the avoidance of doubt, combination and kit-of-parts of the fifth aspect of the invention may have any of the particular features described above for the first, second, third, and fourth aspects of the invention, including all combinations thereof.

In certain embodiments, the combination and kits-of-parts described herein may comprise more than one formulation including an appropriate quantity/dose of a β₂-adrenergic receptor agonist, or pharmaceutically acceptable salt and/or pro drug thereof, and/or more than one formulation including an appropriate quantity/dose of a therapeutic weight loss agent, in order to provide for repeat dosing. If more than one formulation (comprising either active compound) is present, such formulations may be the same, or may be different in terms of the dose of either compound, chemical composition(s) and/or physical form(s).

The skilled person will understand that, in relation to the treatments, formulations, combinations and kits-of-parts as described herein, references to treatment with or administration of each component will refer to said component being administered in conjunction with the other.

### Other Medical Uses

The skilled person will understand that formulations, combinations and kits-of-parts comprising therapeutic weight loss agents, as described herein, may also be used in the treatment of disorders such as obesity and the reduction of body weight, which will be particularly suitable for use in patients having or at risk of developing muscular atrophy, as defined herein.

Thus, in alternative embodiments of the second, third, fourth and fifth aspects of the invention, the pharmaceutical formulation, combination or kit-of-parts is for:
(i) the treatment and/or prophylaxis of obesity; and/or
(ii) lowering body fat composition and/or reducing body weight.

In further alternative embodiments of the second, third, fourth and fifth aspects of the invention, there is provided a method for:
(i) the treatment and/or prophylaxis of obesity; and/or
(ii) lowering body fat composition and/or reducing body weight,
   comprising administering to a patient in need thereof a therapeutically effective amount of the pharmaceutical formulation, combination or kit-of-parts.

As described herein, certain therapeutic weight loss agents will induce weight loss as a secondary effect and will have a primary effect in the treatment of another disease or disorder, such as diabetes.

Thus, in further alternative embodiments of the second, third, fourth and fifth aspects of the invention, where the therapeutic weight loss agent is a therapeutic agent for the treatment of type 1 or type 2 diabetes (e.g. type 2 diabetes), the pharmaceutical formulation, combination or kit-of-parts is for the treatment of type 1 or type 2 diabetes (e.g. type 2 diabetes).

In further alternative embodiments of the second, third, fourth and fifth aspects of the invention, where the therapeutic weight loss agent is a therapeutic agent for the treatment of type 1 or type 2 diabetes (e.g. type 2 diabetes), there is provided a method for the treatment of type 1 or type 2 diabetes (e.g. type 2 diabetes), comprising administering to a patient in need thereof a therapeutically effective amount of the pharmaceutical formulation, combination or kit-of-parts.

For the avoidance of doubt, all embodiments and features of the second, third, fourth and fifth aspects of the invention will apply to the corresponding alternatives.

Thus, in a particular embodiment, the therapeutic weight loss agent is a GLP-1 receptor agonist (which may be understood to also be a therapeutic agent for the treatment of type 1 or type 2 diabetes (e.g. type 2 diabetes)).

In a more particular embodiment, the therapeutic weight loss agent is liraglutide.

Moreover, a particular β₂-adrenergic receptor agonist that may be mentioned is the following compound:

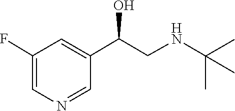

and pharmaceutically acceptable salts thereof.

A further β₂-adrenergic receptor agonist that may be mentioned is the following compound:

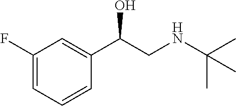

and pharmaceutically acceptable salts thereof.

A further β₂-adrenergic receptor agonist that may be mentioned is the following compound:

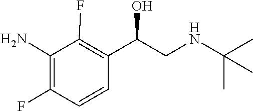 [SMILES: CC(C)(C)NC[C@H](O)c1ccc(F)c(N)c1F]

and pharmaceutically acceptable salts thereof.

In particular embodiments, the
treatment and/or prophylaxis of obesity, and/or lowering body fat composition and/or reducing body weight, or
treatment of type 1 or type 2 diabetes (e.g. type 2 diabetes)
   is in a patient having (i.e. experiencing, such as having been diagnosed with) or at risk of developing muscular atrophy.

In particular embodiments, the treatment and/or prophylaxis of obesity, and/or lowering body fat composition and/or reducing body weight, is the treatment of obesity.

### Methods of Administration

The skilled person will understand that compounds and pharmaceutical formulations as defined herein will normally be administered orally, intravenously, subcutaneously, buccally, rectally, dermally, transdermally, nasally, tracheally, bronchially, sublingually, intranasally, topically, by any other parenteral route or via inhalation, in a pharmaceutically acceptable dosage form. Pharmaceutical formulations as described herein will include compositions in the form of tablets, capsules or elixirs for oral administration, suppositories for rectal administration, sterile solutions or suspensions for parenteral or intramuscular administration, and the like.

In particular embodiments, compounds and pharmaceutical formulations as described herein are administered orally. As such, in certain embodiments pharmaceutical formulations as described herein may be described as oral pharmaceutical formulations.

Thus, in particular embodiments, the pharmaceutical formulation(s) is/are provided in a pharmaceutically acceptable dosage form, including tablets or capsules, liquid forms to be taken orally or by injection, suppositories, creams, gels, foams, transdermal patches, plasters, inhalants (e.g. to be applied intranasally). For the avoidance of doubt, in such embodiments, compounds of the invention may be present as a solid (e.g. a solid dispersion), liquid (e.g. in solution) or in other forms, such as in the form of micelles.

In more particular embodiments, the pharmaceutical formulation(s) is/are provided in a pharmaceutically acceptable oral dosage form, including tablets or capsules, which forms may be prepared using techniques known to those skilled in the art.

For example, in the preparation of pharmaceutical formulations for oral administration, the compound may be mixed with solid, powdered ingredients such as lactose, saccharose, sorbitol, mannitol, starch, amylopectin, cellulose derivatives, gelatin, or another suitable ingredient, as well as with disintegrating agents and lubricating agents such as magnesium stearate, calcium stearate, sodium stearyl fumarate and polyethylene glycol waxes. The mixture may then be processed into granules or compressed into tablets.

Soft gelatin capsules may be prepared with capsules containing one or more active compounds (e.g. compounds of the first and, therefore, second to fifth aspects of the invention, and optionally additional therapeutic agents), together with, for example, vegetable oil, fat, or other suitable vehicle for soft gelatin capsules. Similarly, hard gelatine capsules may contain such compound(s) in combination with solid powdered ingredients such as lactose, saccharose, sorbitol, mannitol, potato starch, corn starch, amylopectin, cellulose derivatives or gelatin.

Dosage units for rectal administration may be prepared (i) in the form of suppositories which contain the compound(s) mixed with a neutral fat base; (ii) in the form of a gelatin rectal capsule which contains the active substance in a mixture with a vegetable oil, paraffin oil, or other suitable vehicle for gelatin rectal capsules; (iii) in the form of a ready-made micro enema; or (iv) in the form of a dry micro enema formulation to be reconstituted in a suitable solvent just prior to administration.

Liquid preparations for oral administration may be prepared in the form of syrups or suspensions, e.g. solutions or suspensions, containing the compound(s) and the remainder of the formulation consisting of sugar or sugar alcohols, and a mixture of ethanol, water, glycerol, propylene glycol and polyethylene glycol. If desired, such liquid preparations may contain colouring agents, flavouring agents, saccharine and carboxymethyl cellulose or other thickening agent. Liquid preparations for oral administration may also be prepared in the form of a dry powder to be reconstituted with a suitable solvent prior to use.

Solutions for parenteral administration may be prepared as a solution of the compound(s) in a pharmaceutically acceptable solvent. These solutions may also contain stabilizing ingredients and/or buffering ingredients and are dispensed into unit doses in the form of ampoules or vials. Solutions for parenteral administration may also be prepared as a dry preparation to be reconstituted with a suitable solvent extemporaneously before use.

The skilled person will understand that the compounds described herein, and formulations and kits-of parts comprising the same, may be administered (for example, as formulations as described hereinabove) at varying doses, with suitable doses being readily determined by one of skill in the art.

In any event, the skilled person (e.g. the physician) will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; however, there can, of course, be individual instances where higher or lower dosage ranges are merited, and such are included within the scope of this invention.

For example, suitable doses of a β₂-adrenergic receptor agonist, or a pharmaceutically acceptable salt thereof, may include those discussed in the above-mentioned publications, as incorporated herein by reference.

Similarly, suitable doses of therapeutic weight loss agents may include those described herein and those known to those skilled in the art (including those indicated in relevant drug formularies, such as the British National Formulary 85^{th} Edition, the contents of which are incorporated herein by reference).

As described herein above, the skilled person will understand that the treatments (and methods of prophylaxis) as described here may further comprise (i.e. be combined with) additional (i.e. other) treatment(s) for the same condition. In particular, treatments (and methods of prophylaxis) described herein may be combined with other means for the treatment of excess body weight or a disoder characterized by excess body weight (as defined herein, such as obesity), such as treatment with one or more other therapeutic agent that is useful in the treatment of excess body weight or a disorder characterized by excess body weight (as defined herein, such as obesity). Similarly, treatments (and methods of prophylaxis) described herein may be combined with other means for the treatment or prophylaxis of muscular atrophy.

Such agents will be readily identified by those skilled in the art and include, in particular, such therapeutic agents that are commercially available (e.g. agents that the subject of a marketing authorization in one or more territory, such as a European or US marketing authorization).

The skilled person will understand that in certain embodiments the present invention (i.e. the pharmaceutical formulations, combinations, kits-of-parts, compounds for use, uses and methods of treatment as described herein, including all embodiments and particular features thereof) relates to two components, namely a β₂-adrenergic receptor agonist, or a pharmaceutically acceptable salt thereof, and a therapeutic weight loss agent being administered in conjunction with each other.

References to each component being administered in conjunction with the other will include the components being administered, sequentially, separately or simultaneously, as part of a medical intervention directed towards treatment of the relevant condition. In particular, such references may include the components being administered sufficiently close in time to enable a beneficial effect for the patient that is greater, over the course of the treatment of the relevant condition, than if administered in the absence of the other component, over the same course of treatment.

Determination of whether a combination provides a greater beneficial effect in respect of, and over the course of, treatment of a particular condition will depend upon the condition to be treated or prevented, but may be achieved routinely by the skilled person.

The skilled person will understand that references to the components (i.e. the β₂-adrenergic receptor agonist, or a pharmaceutically acceptable salt thereof, and the therapeutic weight loss agent) being administered sequentially (and, therefore, separately) will include that individual doses of each component are administered (i.e. taken by the patient, such as being taken orally) within 48 hours (e.g. within 24 hours, 12 hours, 6 hours, 3 hours, 2 hours, 1 hour, 45 minutes, 30 minutes, 20 minutes or 10 minutes) of each other.

Similarly, references to the components being administered simultaneously (which may be in a combined form or separately) will include the components being administered (i.e. taken by the patient, such as being taken orally) at substantially the same time.

In a particular embodiment, the components are administered (i.e. taken by the patient, such as being taken orally) sequentially (and, therefore, as separate doses).

For example, in a particular embodiment, the components are administered sequentially at least 2 hours apart (i.e. the interval between the administration of each component to the patient, e.g. orally, is at least 4 hours), such as between 2 hours and 48 hours apart, or between 2 hours and 24 hours apart, or between 2 hours and 12 hours apart.

For the avoidance of doubt, references to the components as the β₂-adrenergic receptor agonist, or a pharmaceutically acceptable salt thereof, and the therapeutic weight loss agent will include references to the respective components (A) and (B) of the fifth aspect of the invention (i.e. the respective components (A) and (B) of the combination or kit-of-parts of the fifth aspect of the invention).

### Methods of Preparation of Formulations and Kits-of-Parts

Pharmaceutical formulations as described herein may be prepared in accordance with standard and/or accepted pharmaceutical practice.

Thus, in a further aspect of the invention there is provided a process for the preparation of a pharmaceutical composition/formulation, as hereinbefore defined, which process comprises bringing into association a β₂-adrenergic receptor agonist, or a pharmaceutically acceptable salt thereof, with one or more pharmaceutically-acceptable excipients (e.g. an adjuvant, diluent and/or carrier).

In some embodiments, the process comprises bringing into association a β₂-adrenergic receptor agonist, or a pharmaceutically acceptable salt thereof, and a therapeutic weight loss agent, with one or more pharmaceutically-acceptable excipients (e.g. an adjuvant, diluent and/or carrier).

There is further provided a method of preparing a kit-of-parts as defined hereinbefore, which method comprises bringing component (A) into association with component (B), thus rendering the two components suitable for administration in conjunction with each other. As such, references to bringing into association will mean that the two components are rendered suitable for administration in conjunction with each other.

Thus, in relation to the process for the preparation of a kit-of-parts as hereinbefore defined, by bringing the two components “into association with” each other, it is contemplated that the two components of the kit of parts may be:
(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

The skilled person will understand that the uses of compounds and pharmaceutical formulations as described herein, and methods relating to the same, may have the advantage that, in the treatment of the conditions mentioned hereinbefore, they may be more convenient for the physician and/or patient than, be more efficacious than, be less toxic than, have a broader range of activity than, be more potent than, produce fewer side effects than, or may have other useful pharmacological properties over, similar methods (treatments) known in the prior art whether for use in the above-stated indications or otherwise. In particular, such pharmaceutical formulations, kits of parts, methods and uses may have the advantage that they are more efficacious and/or exhibit advantageous properties in vivo.

Without wishing to be bound by theory, it is believed that β₂-adrenergic receptor agonists as described herein may provide potent in vivo effects allowing for effective treatment of muscular dystrophy. In particular, such effects may be even more effectively delivered by compounds that activate the β₂-adrenergic receptor without significantly increasing cAMP, such as compounds A, B and C, thus allowing for treatment without significant levels of adverse events resulting from increased cAMP. Further, such effects may be delivered by compounds that do not indue significant recruitment of beta-arrestin, thus preventing β₂-AR internalization and resulting desensitization.

Without wishing to be bound by theory, it is believed that the use a β₂-adrenergic receptor agonist allows for the prevention and/or treatment of muscular atrophy, in particular the loss of lean muscle mass, that may arise in patients undergoing weight loss treatment, such as weight loss treatment with a therapeutic weight loss agent.

Notably, it has been previously thought that the beneficial effect on muscle mass observed with R-agonists is mediated via a cAMP-dependent pathway. However, it has now been surprisingly found that certain β₂-AR agonists that do not cause a significant release of cAMP are also able to deliver beneficial effects on muscle mass (such as treating muscular atrophy or preventing muscle loss). Importantly, such β₂-AR agonists allow for treatment without significant levels of adverse events resulting from increased cAMP.

Advantageously, the compounds of the invention may produce a “repartitioning effect” by inducing an increase in lean tissue and decrease in body fat. Moreover, the compounds of the invention may lead to improvements in the quality of the muscle tissue (such as improvements in organisation of the muscular fibres and/or reduction of fat tissue embedded in the muscle tissue).

### BRIEF DESCRIPTION OF THE FIGURES

FIGS. 1a to 1c show that the glucose uptake promoted by Compounds A, B and C, respectively, is inhibited in a dose-dependent manner by the selective β₂-adrenergic receptor antagonist ICI-118551.

FIGS. 2a to 2d show that Compounds A, B, and C give significantly less cAMP formation compared to isoprenaline, whereas all four compounds give full glucose uptake (GU).

FIGS. 3a to 3c show that Compounds A, B, and C, respectively, recruit significantly less β-arrestin 2 compared to isoprenaline.

FIG. 4 shows that Compound A reverses dexamethasone induced reduction of body weight in mice.

FIG. 5 shows that Compound A reverses dexamethasone induced reduction of lean mass in mice.

FIG. 6 shows the organization of muscular fibres in a wild-type sibling control zebrafish embryo (with dystrophin protein, upper image) and dystrophin (−/−) K.O. zebrafish embryo (bottom image).

FIGS. 7a and 7b shows the effect of increasing concentrations of salbutamol relative to control.

FIGS. 8a and 8b shows the effect of increasing concentrations of Compound A relative to control.

FIGS. 9a and 9b show cumulative food intake for Compounds B and C, respectively, over the period of the experiment described in Biological Examples 7 and 8.

FIGS. 10a and 10b show the change in body weight for Compounds B and C, respectively, over the period of the experiment described in Biological Examples 7 and 8.

FIGS. 11a and 11b show the change in fat mass for Compounds B and C, respectively, over the period of the experiment described in Biological Examples 7 and 8.

FIGS. 12a and 12b show the change in lean mass for Compounds B and C, respectively, over the period of the experiment described in Biological Examples 7 and 8.

### EXAMPLES

The present invention is illustrated by way of the following examples, which are not intended to be limiting on the overall scope of the invention.

For the avoidance of doubt, in the case of a discrepancy between the name of the compound and the structure drawn in this specification, the structure should prevail.

The synthesis of (R)-2-(tert-butylamino)-1-(3-fluorophenyl)ethan-1-ol and biological testing thereof is described in WO 2019/053426 (see, e.g., Example 5 therein), the contents of which are incorporated herein by reference.

The synthesis of (R)-2-(tert-butylamino)-1-(5-fluoropyridin-3-yl)ethan-1-ol dihydrochloride and biological testing thereof is described in WO 2019/053427 (see, e.g., Example 17 therein), the contents of which are incorporated herein by reference. The preparation of the corresponding hemi-tartrate (Compound B) is described in WO 2023/105035, the contents of which are incorporated herein by reference.

The synthesis of (R)-2-(tert-butylamino)-1-(3-fluorophenyl)ethan-1-o and biological testing thereof is described in WO 2019/053426 (see, e.g., Example 31 therein), the contents of which are incorporated herein by reference.

### EXAMPLE COMPOUNDS

### Compound Example 1

### Compound A: (R)-2-(tert-Butylamino)-1-(3-fluorophenyl)ethan-1-ol hydrochloride

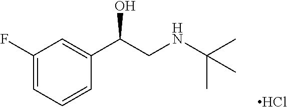

### (a) (R)-2-Bromo-1-(3-fluorophenyl)ethan-1-ol

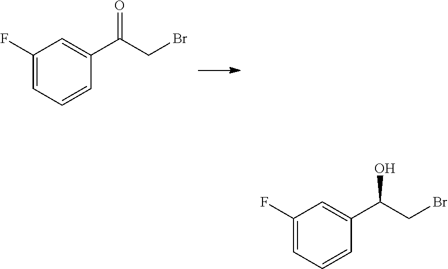 [SMILES: O=C(CBr)c1cccc(F)c1 ; O[C@@H](CBr)c1cccc(F)c1]

Borane (1 M in THF, 0.68 mL, 0.68 mmol) was added dropwise to a mixture of (R)-2-methyl-CBS-oxazaborolidine (1 M in toluene, 0.85 mL, 0.85 mmol) and tetrahydrofuran (THF; 0.8 mL) at room temperature (rt). The mixture was stirred 15 min at rt and a solution of 3-fluorophenacyl bromide (185 mg, 0.85 mmol) in THF (1.9 mL) was added dropwise (0.09 mL/min). After 6 h at rt, MeOH (10 mL) was added. The mixture was stirred for 30 min and concentrated. Purification by chromatography gave the sub-title compound (150 mg, 0.68 mmol, 80%).

### (b) (R)-2-(3-Fluorophenyl)oxirane

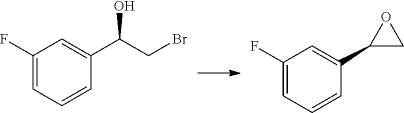 [SMILES: O[C@@H](CBr)c1cccc(F)c1 ; Fc1cccc([C@@H]2CO2)c1]

K₂CO₃ (137 mg, 0.99 mol) was added to a mixture of (R)-2-bromo-1-(3-fluorophenyl)-ethan-1-ol (145 mg, 0.66 mmol) in MeOH (6.8 mL) at rt. The mixture was stirred for 30 min, filtered and concentrated. The residue was extracted with CH₂Cl₂. The combined extracts were concentrated to give the sub-title compound (70 mg, 0.51 mmol, 77%), which was used in the next step without further purification.

### (c) (R)-2-(tert-Butylamino)-1-(3-fluorophenyl)ethan-1-ol

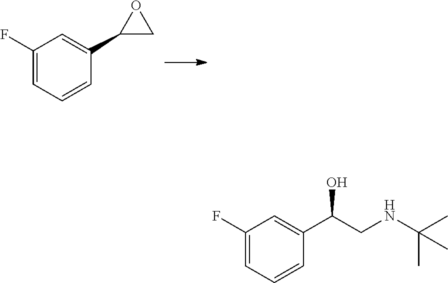

A mixture of (R)-2-(3-fluorophenyl)oxirane (30 mg, 0.22 mmol), tert-butylamine (66 mg, 0.90 mmol) and MeOH (0.2 mL) was stirred at reflux for 16 h, cooled and concentrated and dissolved in a Et₂O. Et₂O/pentane (1:3) was added and the solution was kept at −20° C. overnight. The solid formed was collected to give the title compound (25 mg, 0.12 mmol, 54%).

¹H NMR (400 MHz, CDCl₃): δ 7.33-7.26 (m, 1H), 7.14-7.09 (m, 2H), 6.98-6.93 (m, 1H), 4.57 (dd, J=8.4, 3.6 Hz, 1H), 2.92 (dd, J=12.0, 4.0 Hz, 1H), 2.55 (dd, J=12.0, 8.4 Hz, 1H), 1.10 (s, 9H).

The compound was used in the biological examples described herein in the form of the HCl salt thereof (obtained using standard techniques as known to those skilled in the art), referred to herein as Compound A.

### Compound Example 2

### Compound B: (R)-2-(tert-Butylamino)-1-(5-fluoropyridin-3-yl)ethan-1-ol hemi-tartrate

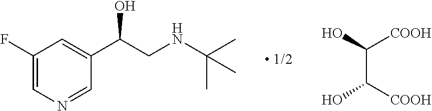

### (a) 2-Chloro-1-(5-fluoropyridin-3-yl)ethan-1-one

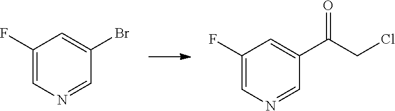 [SMILES: Fc1cncc(Br)c1 ; O=C(CCl)c1cncc(F)c1]

Isopropylmagnesium chloride (2 M in THF, 10.47 mL, 20.94 mmol) was added to a solution of LiCl (887.69 mg, 20.94 mmol) in THF (8 mL) at rt. After 15 min at rt, 3-bromo-5-fluoropyridine (3.35 g, 19.04 mmol) in THF (30 mL) was added dropwise at 0° C. The mixture was stirred at rt for 2 h and cooled in an ice-bath. A solution of 2-chloro-N-methoxy-N-methylacetamide (2.62 g, 19.04 mmol) in THF (30 mL) was added dropwise, and the mixture was stirred at rt for 2 h. NH₄Cl (aq, 10%) was added and the mixture was extracted with Et₂O. The combined extracts were washed with brine, dried (Na₂SO₄) and concentrated. The residue was purified by chromatography to give the sub-title compound (1.52 g, 20.94 mmol, 46%).

### (b) (R)-2-Chloro-1-(5-fluoropyridin-3-yl)ethan-1-ol

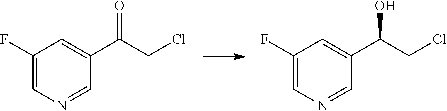 [SMILES: O=C(CCl)c1cncc(F)c1 ; O[C@@H](CCl)c1cncc(F)c1]

RhClCp*[(1S,2S)-p-TsNCH(C₆H₅)CH(C₆H₅)NH₂]/HCl·Et₃N (68.0 mg, 87.6 μmol), prepared from dichloro(pentamethylcyclopentadienyl)rhodium(III) dimer, (1S,2S)-(+)-N-(4-toluenesulphonyl)-1,2-diphenylethylene diamine and Et₃N as described in WO 2008/054155, was added to a mixture of 2-chloro-1-(5-fluoropyridin-3-yl)ethan-1-one (1.52 g, 8.76 mmol) in DMF (75 mL). Formic acid/Et₃N (5:2, 25 mL) was added and the mixture was stirred at rt for 15 min. H₂O (60 mL) and EtOAc (60 ml) where added and the layers were separated. The aq layer was washed with EtOAc (2×60 mL) and the combined organic phases were washed with H₂O, dried (Na₂SO₄), filtered and concentrated. The residue was purified by chromatography to give the sub-title compound (1.35 g, 7.69 mmol, 88%, ee=92.5%).

### (c) (R)-2-(tert-Butylamino)-1-(5-fluoropyridin-3-yl)ethan-1-ol

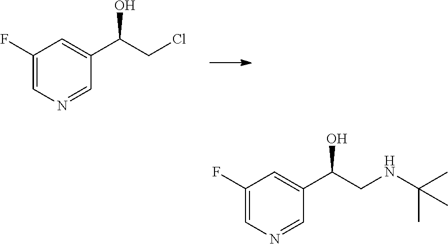

tert-Butylamine (11.37 mL, 108.21 mmol) followed by NaOH (476.08 mg, 11.90 mmol) were added to a mixture of 2-chloro-1-(5-fluoropyridin-3-yl)ethan-1-one (1.90 g, 10.82 mmol) and iPrOH (1.66 mL, 21.64 mmol) at rt. The mixture was heated at 75° C. for 4 h, allowed to cool, diluted with EtOAc, washed with H₂O and brine, dried (Na₂SO₄) and concentrated. The residue was dissolved in hot EtOAc and allowed to cool. Pentane was added and the mixture kept at −20° C. overnight. The solids were collected and purified by chromatography to give the title compound (1.43 g, 6.74 mmol, 62%, ee=98%).

¹H NMR (400 MHz, CDCl₃): δ 8.43-8.27 (m, 2H), 7.57-7.42 (m, 1H), 4.62 (dd, J=8.8, 3.7 Hz, 1H), 2.94 (dd, J=12.1, 3.8 Hz, 1H), 2.53 (dd, J=12.1, 8.8 Hz, 1H), 1.10 (s, 9H).

### (d) (R)-2-(tert-Butylamino)-1-(5-fluoropyridin-3-yl)ethan-1-ol hemi-tartrate

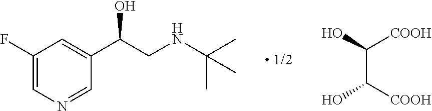

A solution of L-(+)-tartaric acid (6.21 g, 0.5 eq) in EtOH (175 mL) was added to a solution of (R)-2-(tert-butylamino)-1-(5-fluoropyridin-3-yl)ethan-1-ol (17.57 g) in EtOH (525 mL, 30 vol) and H₂O (7 mL) at rt. The mixture was refluxed until all precipitate was dissolved, then cooled. The resultant slurry was stirred at rt overnight and then at 0 to 5° C. for 2 h. The solids were collected to give the title salt of (R)-2-(tert-butylamino)-1-(5-fluoropyridin-3-yl)ethan-1-ol (19.9 g, 83%, 100.0% purity by HPLC, 99.8% ee by HPLC).

Formation of the hemi-tartrate was confirmed by 1H-NMR spectrum, which indicated an amine:acid ratio of 2:1.

The compound was used in the biological examples described herein, referred to herein as Compound B.

### Compound Example 3

### Compound C: (R)-1-(3-Amino-2,4-difluorophenyl)-2-(tert-butylamino)ethan-1-ol maleate

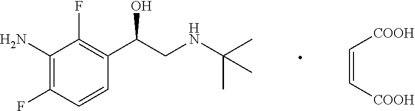 [SMILES: CC(C)(C)NC[C@H](O)c1ccc(F)c(N)c1F ; "·" ; O=C(O)/C=C\C(=O)O]

### (a) N-(2,6-Difluoro-3-methylphenyl)acetamide

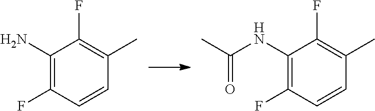

The sub-title compound was prepared from 2,6-difluoro-3-methylaniline in accordance with the procedure in Example 6, Step (a) of WO 2019/053426.

### (b) 3-Acetamido-2,4-difluorobenzoic Acid

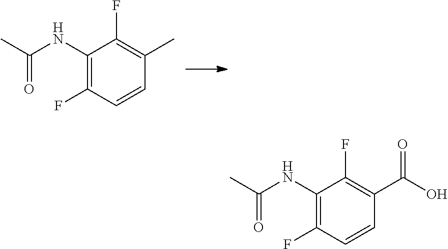

The sub-title compound was prepared from N-(2,6-difluoro-3-methylphenyl)acetamide in accordance with the procedure in Example 7, Step (b) of WO 2019/053426.

### (c) N-(3-(2-Chloroacetyl)-2,6-difluorophenyl)acetamide

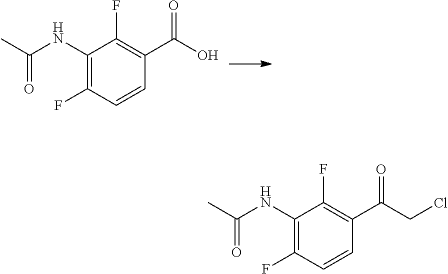 [SMILES: CC(=O)Nc1c(F)ccc(C(=O)O)c1F ; CC(=O)Nc1c(F)ccc(C(=O)CCl)c1F]

A mixture of 3-acetamido-2,4-difluorobenzoic acid (250 mg, 1.16 mmol) and SOCl₂ (2.6 mL) was heated at 60° C. for 4 h and allowed to cool. Toluene was added and the mixture concentrated. The procedure of adding toluene followed by concentration was repeated three times. The residue was dissolved in CH₂Cl₂ and trimethylsilyl diazomethane (1.16 mL, 2.32 mmol) was added dropwise at 0° C. The mixture was allowed to come to rt over 18 h and cooled to 0° C. HCl (4 M in dioxane, 1.45 mL, 5.81 mmol) was added dropwise. The mixture was allowed to come to rt over 1 h, diluted with EtOAc and washed with Na₂CO₃ (aq, sat), dried over MgSO₄ and concentrated. The residue was purified by chromatography to give the sub-title compound (198 mg, 0.80 mmol, 69%).

### (d) (R)—N-(3-(2-Chloro-1-hydroxyethyl)-2,6-difluorophenyl)acetamide

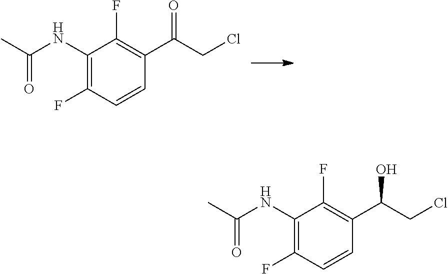 [SMILES: CC(=O)Nc1c(F)ccc(C(=O)CCl)c1F ; CC(=O)Nc1c(F)ccc([C@@H](O)CCl)c1F]

RhClCp*[(1S,2S)-p-TsNCH(C₆H₅)CH(C₆H₅)NH₂]/HCl·Et₃N (5.02 mg, 0.0065 mmol), prepared from dichloro(pentamethylcyclopentadienyl)rhodium (III) dimer, (1S, 2S)-(+)-N-(4-toluenesulphonyl)-1,2-diphenylethylene diamine and Et₃N as described in WO 2008/054155, was added to a mixture of N-(3-(2-chloroacetyl)-2,6-difluorophenyl)acetamide (160 mg, 0.65 mmol) in DMF (2.7 mL). Formic acid/Et₃N (5:2, 0.90 mL) was added and the mixture was stirred at rt for 20 min. The mixture was diluted with EtOAc, washed with H₂O and brine, dried (Na₂SO₄), filtered and concentrated. The residue was crystallized from CH₂Cl₂/hexane to give the sub-title compound (101 mg, 0.41 mmol, 63%, ee=97%).

### (e) (R)—N-(3-(2-(tert-Butylamino)-1-hydroxyethyl)-2,6-difluorophenyl)acetamide

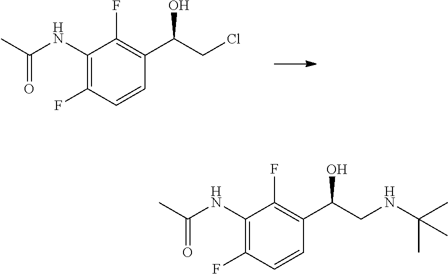 [SMILES: CC(=O)Nc1c(F)ccc([C@@H](O)CCl)c1F ; CC(=O)Nc1c(F)ccc([C@@H](O)CNC(C)(C)C)c1F]

The sub-title compound was prepared from (R)—N-(3-(2-chloro-1-hydroxyethyl)-2,6-difluorophenyl)acetamide in accordance with the procedure in Example 25, Step (d) of WO 2019/053426.

### (f) (R)-1-(3-Amino-2,4-difluorophenyl)-2-(tert-butylamino)ethan-1-ol hydrochloride

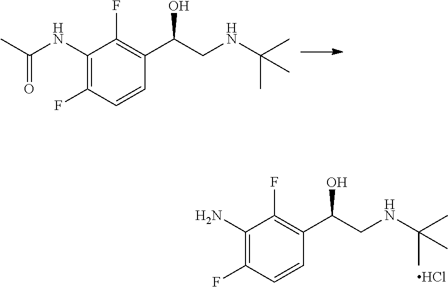 [SMILES: CC(=O)Nc1c(F)ccc([C@@H](O)CNC(C)(C)C)c1F ; CC(C)(C)NC[C@H](O)c1ccc(F)c(N)c1F ; Cl]

NaOH (aq, 10%, 0.52 mL) was added to of (R)—N-(3-(2-(tert-butylamino)-1-hydroxy-ethyl)-2,6-difluorophenyl)acetamide (52 mg, 0.18 mmol) in EtOH (0.52 mL) and the mixture was heated at 75° C. for 20 h. The EtOH was removed in vacuo and the residue extracted with CH₂Cl₂. The combined extracts were washed with water, dried over Na₂SO₄, filtered and concentrated. The residue was dissolved in Et₂O. HCl (2 M in Et₂O, 0.13 mL, 0.27 mmol) was added. The solids were collected and dried to give the sub-title compound (32 mg, 0.11 mmol, 63%).

¹H NMR (400 MHz, D₂O): δ 7.11-7.01 (m, 2H), 5.29-5.15 (dd, J=9.6, 3.2 Hz, 1H), 3.36-3.23 (m, 2H), 1.40 (s, 9H).

### (g) (R)-1-(3-Amino-2,4-difluorophenyl)-2-(tert-butylamino)ethan-1-ol maleate

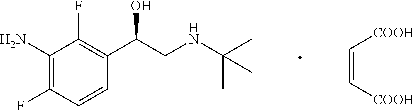 [SMILES: CC(C)(C)NC[C@H](O)c1ccc(F)c(N)c1F ; "·" ; O=C(O)/C=C\C(=O)O]

A solution of maleic acid (43 mg, 0.37 mmol) in iPrOH (0.4 mL) was added to a solution of (R)-1-(3-amino-2,4-difluorophenyl)-2-(tert-butylamino)ethan-1-ol (98 mg, 0.40 mmol, free base, prepared as in Step (f)) in iPrOH (1.5 mL) at rt. The mixture was stirred at rt for 1 h and centrifuged. The supernatant was removed and the residue was dried, crystallized from EtOAc and dried to give the title compound (71 mg, 0.20 mmol, 50%).

Formation of the maleate was confirmed by 1H-NMR spectrum, which indicated an amine:acid ratio of 1:1

The compound was used in the biological examples described herein referred to herein as Compound C.

### BIOLOGICAL EXAMPLES

### Biological Example 1: Glucose Uptake in the Presence of a Selective β₂-Adrenergic Receptor Inhibitor

L6-myoblasts were grown in Dulbecco's Modified Eagle's Medium (DMEM) containing 1 g/L glucose supplemented with 10% fetal bovine serum (FBS), 2 mM L-glutamine, 50 U/mL penicillin, 50 μg/mL streptomycin and 10 mM HEPES. Cells were plated at 1×10⁵ cells per mL in 24-well plates. After reaching 90% confluence the cells were grown in medium containing 2% FBS for 7 days where upon cells differentiated into myotubes. The differentiated L6-myotubes were serum-starved overnight in medium containing 0.5% fatty-acid free BSA and stimulated with the test compound at a final concentration of 1×10⁻⁵ M in the presence of different concentrations of the selective β₂-adrenergic receptor antagonist ICI-118551. After 1 h 40 min the cells were washed with warm, glucose free medium twice and another portion of agonist was added to the glucose free medium. After another 20 min of incubation the cells were exposed to 50 nM ³H-2-deoxyglucose for 10 min before washed in ice cold glucose free medium three times and lysed in 400 μL/well 0.2 M NaOH for 1 h at 60° C. The cell lysate was mixed with 4 ml scintillation buffer (Emulsifier Safe, Perkin Elmer) and the radioactivity was detected in a β-counter (Tri-Carb 4810TR, Perkin Elmer).

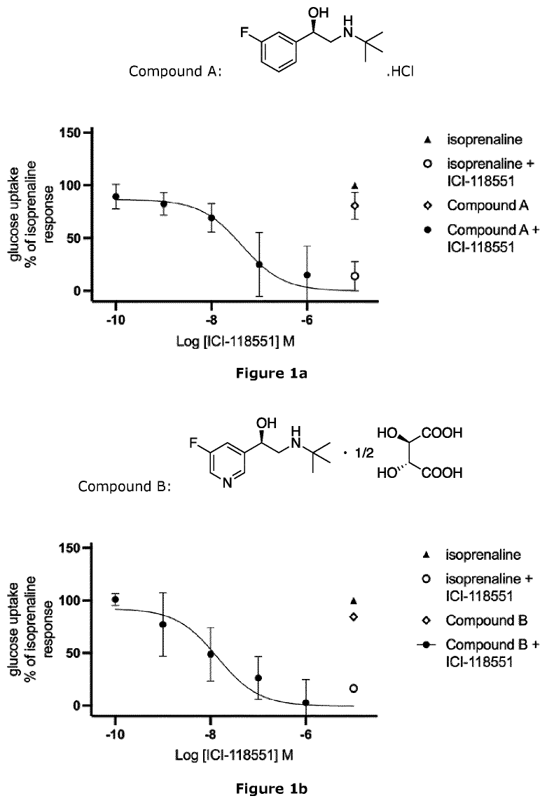

The results are presented in FIGS. 1a, 1b and 1c, which show that the glucose uptake promoted by Compounds A, B, and C, respectively, are inhibited in a dose-dependent manner by the selective β₂-adrenergic receptor antagonist ICI-118551.

### Biological Example 2: Glucose Uptake

Differentiated L6-myotubes were serum-starved overnight in medium containing 0.5% fatty-acid free BSA and stimulated with isoprenaline or the test compound, with a final concentration of 1×10⁻⁵ M. After 1 h 40 min the cells were washed with warm glucose free medium or PBS twice and another portion of agonist was added to the glucose free medium. After 20 min the cells were exposed to 50 nM ³H-2-deoxyglucose for 10 min before washed with ice cold glucose free medium or PBS three times and lysed with 0.2 M NaOH, 400 μL/well, for 1 h at 60° C. The cell lysate was mixed with 4 mL scintillation buffer (Emulsifier Safe, Perkin Elmer) and the radioactivity was detected in a 3-counter (Tri-Carb 4810TR, Perkin Elmer).

The results for various compounds tested are given in Table 1.

### Biological Example 3: Measurement of Intracellular cAMP Levels

Differentiated L6-myotubes were serum-starved overnight and stimulated with isoprenaline or the test compound with a final concentration of 1×10⁻⁵ M, for 15 min in stimulation buffer (HBSS supplemented with 1% BSA, 5 mM HEPES and 1 mM IBMX, pH 7.4). The medium was aspirated and 100 μL of 95% EtOH was added to each well of the 24-well plate and cells were kept at −20° C. overnight. The EtOH was allowed to evaporate and 500 μL of lysis buffer (1% BSA, 5 mM HEPES and 0.3% Tween-20, pH 7.4) was added to each well. The plate was kept at −80° C. for 30 min and then at −20° C. until the day of detection when the samples were thawed. Intracellular cAMP levels were detected using an alpha screen cAMP kit (6760635D from Perkin Elmer).

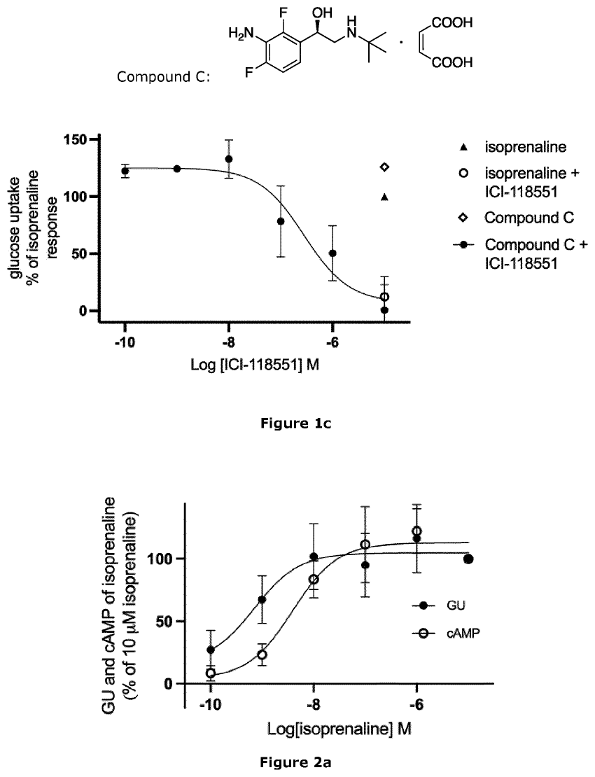

The results are presented in FIG. 2a, which shows the glucose uptake and the intracellular cAMP levels when cells are treated with isoprenaline; FIG. 2b, which shows the glucose uptake and the intracellular cAMP levels when cells are treated with Compound A; FIG. 2c, which shows the glucose uptake and the intracellular cAMP levels when cells are treated with Compound B; and FIG. 2d, which shows the glucose uptake and the intracellular cAMP levels when cells are treated with Compound C; showing that isoprenaline and Compounds A, B and C, all give a full glucose uptake response, whereas Compounds A, B and C, in contrast to isoprenaline, only exhibits low intracellular cAMP levels.

Additional results for other compounds tested are given in Table 1.

### Biological Example 4: Measurement of β-Arrestin Recruitment

HEK293 cells are transfected with both the β₂-adrenergic receptor attached with a Large BiT SNAP-tag protein, and with p-arrestin 2 attached to a Small BiT SNAP-tag protein. When cells are stimulated and the receptor recruits β-arrestin 2, the Small- and Large-BiT proteins merge, forming a functional luciferase enzyme, that in presence of furimazine generates light. After 31 min, the light is measured with a luminescence plate reader.

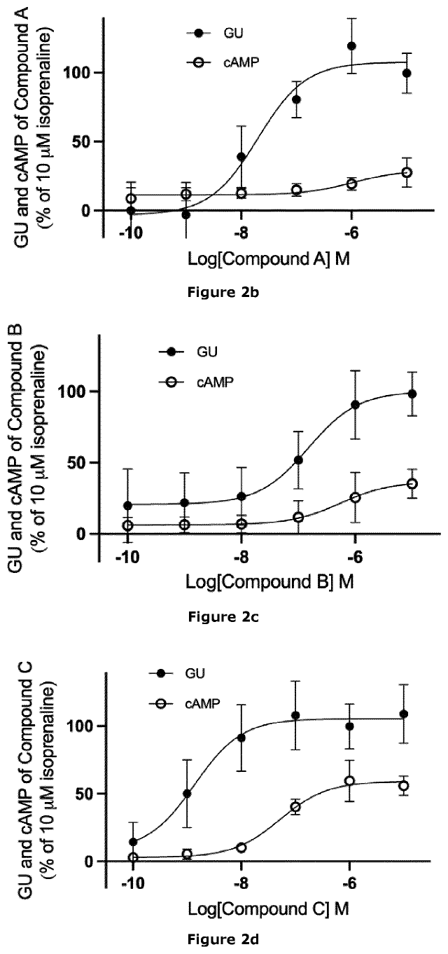

The results are presented in FIG. 3a, which shows the difference between the p-arrestin 2 recruitment of isoprenaline and Compound A; FIG. 3b, which shows the difference between the p-arrestin 2 recruitment of isoprenaline and Compound B; and FIG. 3c, which shows the difference between the p-arrestin 2 recruitment of isoprenaline and Compound C, showing that, in contrast to isoprenaline, Compounds A, B and C only give minimal p-arrestin 2 recruitment.

### Biological Example 5: Effects on Body Weight and Lean Mass when Compound A is Administered to Mice Treated with Dexamethasone

40 eight-week-old male C57Bl/6 mice kept on standard chow diet were grouped-caged (2-3 mice per cage) at 21° C. The mice had similar body weight and body composition and were divided into four groups (n=10) and were treated daily with subcutaneous injections for 3 weeks with either dexamethasone sodium phosphate (10 mg/kg) or Compound A (47.1 μmol/kg (corresponding to dose 10 mg/kg of the free base)), or a combination of dexamethasone and Compound A. Control mice received saline. Body weight and lean mass were measured after 6, 12 and 20 days of treatment.

The results are presented in:

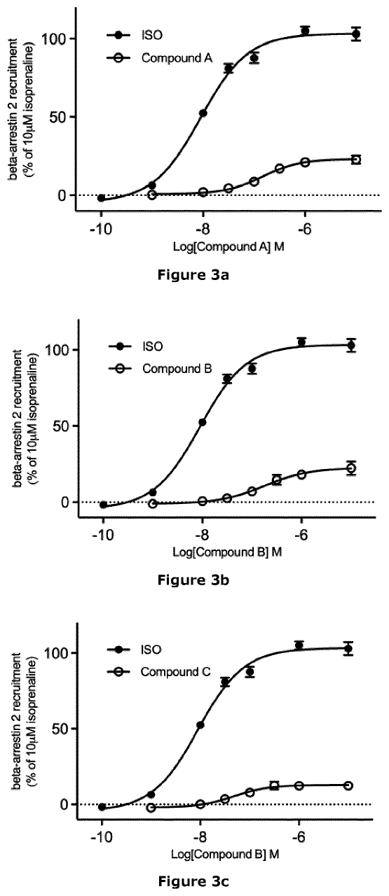

FIG. 4, which shows that mice lose weight compared to control when treated with dexamethasone and that the effect is reversed when they are treated simultaneously with Compound A; and

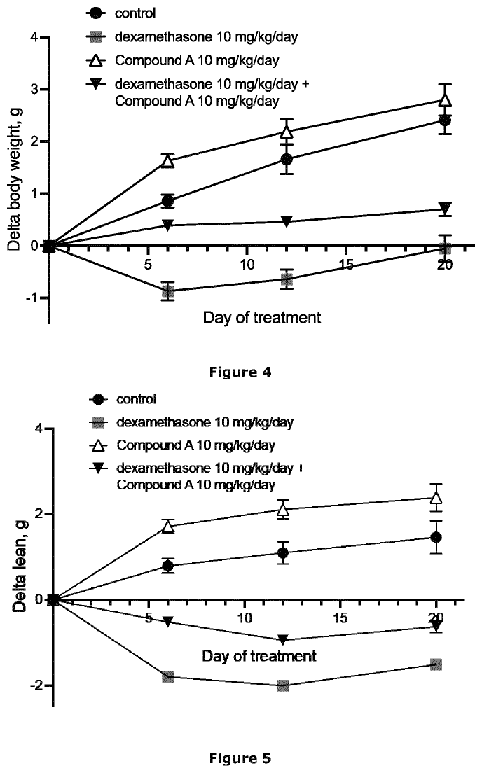

FIG. 5, which shows that mice lose lean mass compared to control when treated with dexamethasone and that the effect is reversed when they are treated simultaneously with Compound A.

### Biological Example 6: Zebra Fish Model

Sepje zebrafish lacks the dystrophin gene, and such as in humans, lack of dystrophin leads to a progressive disorganization of muscular fibres and subsequent loss of muscle mass. The sepje model is equivalent to the MDX mouse, which also lacks the dystrophin gene.

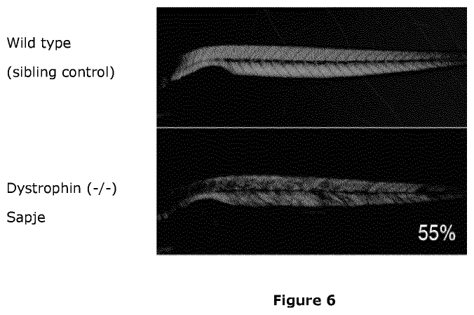

FIG. 6 shows the organization of muscular fibres in a wild-type sibling control zebrafish embryo (with dystrophin protein, upper image) and dystrophin (−/−) K.O. zebrafish embryo (bottom image), measured by birefringence microscopy.

Lacking dystrophin results in poor fibre organization, and sepje often begin to die at 4-5 days post fertilization (dpf). Wild-type or dystrophin (−/−) embryos were treated from 1 to 5 dpf with vehicle control or increasing concentrations of salbutamol or Compound A (10, 30, and 100 μM).

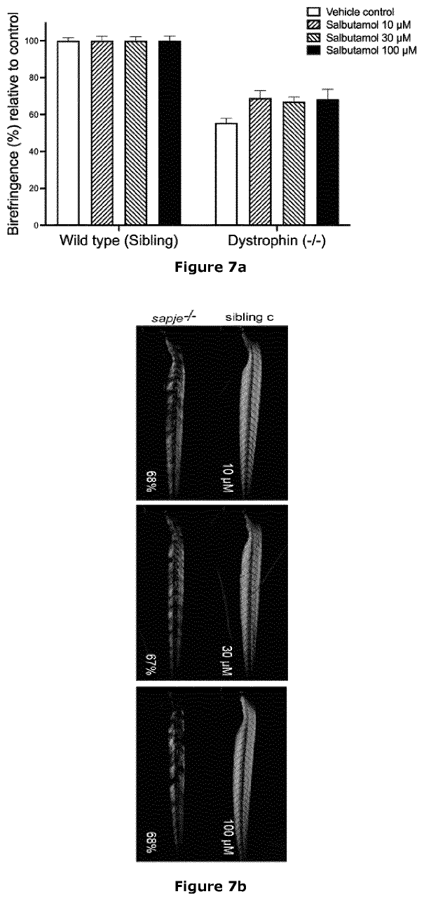

FIG. 7a shows the effect of increasing concentrations of salbutamol on birefringence relative to control. FIG. 7b shows the effect of increasing concentrations of salbutamol on the organization of muscular fibres, measured by birefringence microscopy.

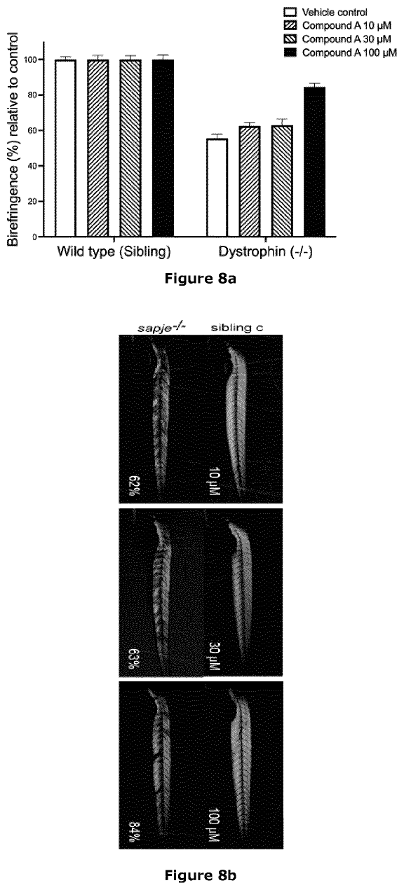

FIG. 8a shows the effect of increasing concentrations of Compound A on birefringence relative to control. FIG. 8b shows the effect of increasing concentrations of Compound A on the organization of muscular fibres, measured by birefringence microscopy.

### Biological Example 7: Combination of Compound B and Liraglutide

2.5 Months old male C57Bl/6N mice were grouped caged (4-5 mice per cage), kept at 30° C. and fed with high-fat, high-sucrose diet (45% fat) for 5.5 months. The mice were divided into four groups (2-3 mice per cage) according to their fasting blood glucose levels, glucose tolerance, body weight, body fat and lean mass, and treated daily for 2 weeks, subcutaneously with either vehicle, liraglutide (0.1 mg/kg), Compound B (1.4 mg/kg), or a mixture of liraglutide (0.1 mg/kg) and Compound A (1.4 mg/kg). Food intake was measured twice a week; body weight, body fat and lean mass were measured weekly.

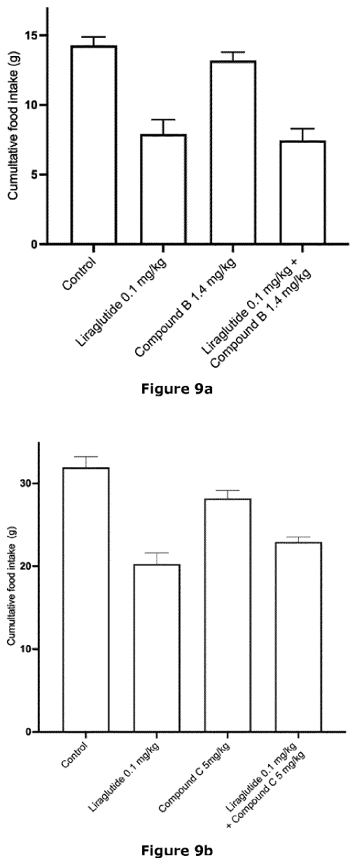

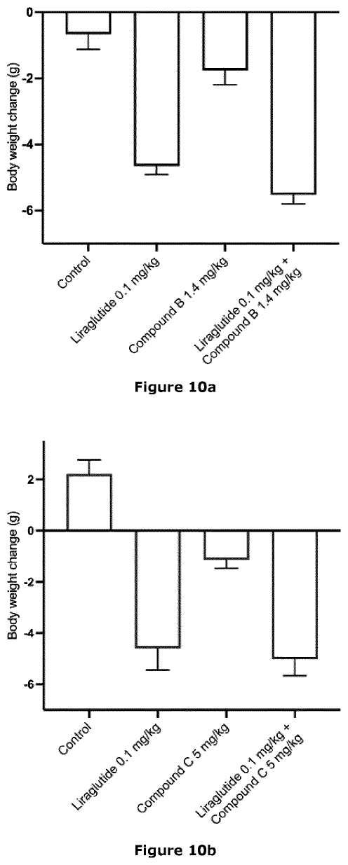

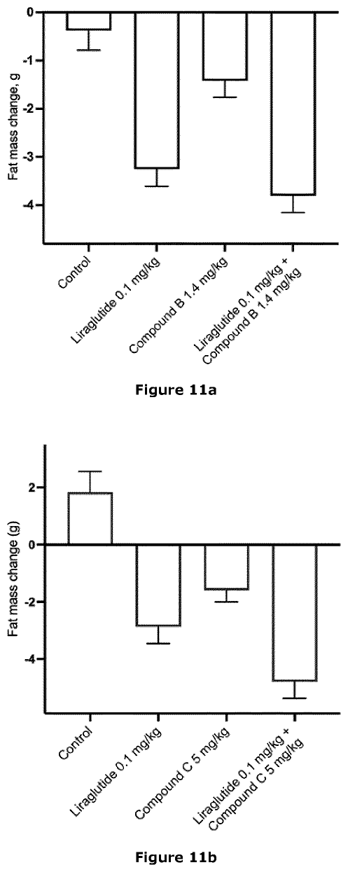

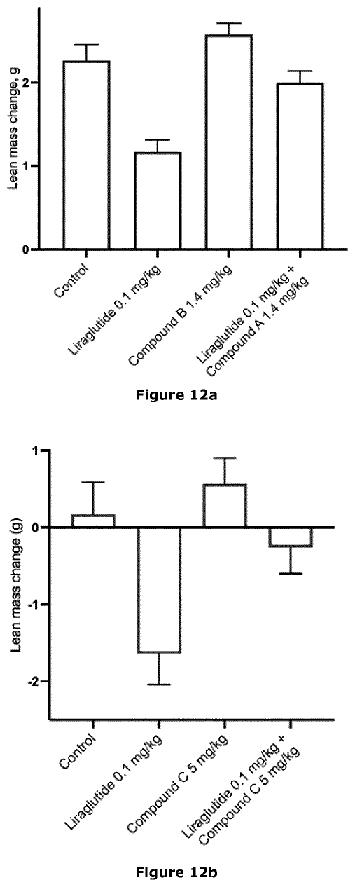

The results obtained over the period of the experiment are presented as follows: FIG. 9a shows cumulative food intake; FIG. 10a shows the change in body weight; FIG. 11a shows the change in fat mass; FIG. 12a shows the change in lean mass.

These data confirm that administration of liraglutide, and a combination of liraglutide and Compound B, respectively, reduced food intake as compared to the control. Further, both liraglutide, and a combination of liraglutide and Compound B, resulted in a significant reduction of body weight and fat mass. However, the change in lean mass for the combination of liraglutide and Compound B was significantly lower as compared to administration of liraglutide alone.

### Biological Example 8: Combination of Compound C and Liraglutide

2.5 Months old male C57Bl/6N mice were grouped caged (4-5 mice per cage), kept at 30° C. and fed with high-fat, high-sucrose diet (45% fat) for 5 months. The mice were divided into four groups (2-3 mice per cage) according to their fasting blood glucose levels, glucose tolerance, body weight, body fat and lean mass, and treated daily for 2 weeks, subcutaneously with either vehicle, liraglutide (0.1 mg/kg), Compound C (5 mg/kg), or a mixture of liraglutide (0.1 mg/kg) and Compound C (5 mg/kg). Food intake and body weight were measured every other day; body fat and lean mass were measured weekly.

The results obtained over the period of the experiment are presented as follows: FIG. 9b shows cumulative food intake; FIG. 10b shows the change in body weight; FIG. 11b shows the change in fat mass; FIG. 12b shows the change in lean mass.

These data confirm that administration of liraglutide, and a combination of liraglutide and Compound C, respectively, reduced food intake as compared to the control. Further, all three treatments resulted in a significant reduction of body weight and fat mass. However, the change in lean mass for the combination of liraglutide and Compound C, as well as Compound C alone, was significantly lower as compared to administration of liraglutide alone.

1. A β₂-adrenergic receptor agonist, or a pharmaceutically acceptable salt thereof, for use in the treatment or prophylaxis of a disease or disorder characterised by muscle wasting.

2. A method for the treatment or prophylaxis of a disease or disorder characterised by muscle wasting, comprising administration of a therapeutically effective amount of a β₂-adrenergic receptor agonist, or a pharmaceutically acceptable salt thereof, to a patient in need thereof.

3. The use of a β₂-adrenergic receptor agonist, or a pharmaceutically acceptable salt thereof, in the manufacture of a medicament for the treatment or prophylaxis of a disease or disorder characterised by muscle wasting.

4. A pharmaceutical formulation comprising a β₂-adrenergic receptor agonist, or a pharmaceutically acceptable salt thereof, and optionally one or more pharmaceutically acceptable excipient, for use in the treatment or prophylaxis of a disease or disorder characterised by muscle wasting.

5. The β₂-adrenergic receptor agonist for use, method, use or formulation for use of any of claims 1 to 4, wherein the disease or disorder characterised by muscle wasting is muscular dystrophy, such as Duchenne muscular dystrophy.

6. The β₂-adrenergic receptor agonist for use, use, method or formulation for use of any one of claims 1 to 5, wherein the treatment and/or prophylaxis of muscle wasting is treatment and/or prophylaxis of muscular atrophy in a patient undergoing weight loss.

7. The β₂-adrenergic receptor agonist for use, use, method, or formulation for use of claim 6, wherein treatment and/or prophylaxis of muscular atrophy is the treatment and/or prophylaxis of loss of lean mass.

8. The β₂-adrenergic receptor agonist for use, use, method, or formulation for use of any one of claims 6 and 7, wherein the weight loss is weight loss treatment.

9. The β₂-adrenergic receptor agonist for use, use, method or formulation for use of claim 8, wherein the weight loss treatment comprises treatment and/or prophylaxis with one or more therapeutic weight loss agent for:
(i) the treatment and/or prophylaxis of obesity; and/or
(ii) lowering body fat composition and/or reducing body weight.

10. The β₂-adrenergic receptor agonist for use, use, method or formulation for use of claim 9, wherein the weight loss treatment comprises treatment with one or more therapeutic weight loss agent, such as a therapeutic weight loss agent for the treatment or prophylaxis of obesity.

11. The β₂-adrenergic receptor agonist for use, use, method or formulation for use of claim 10, wherein the therapeutic weight loss agent is a GLP-1 receptor agonist.

12. The β₂-adrenergic receptor agonist for use, use, method or formulation for use of claim 11, wherein the GLP-1 receptor agonist is liraglutide.

13. A pharmaceutical formulation comprising:
(a) a β₂-adrenergic receptor agonist, or a pharmaceutically acceptable salt thereof; and
(b) a therapeutic weight loss agent,
and optionally one or more pharmaceutically acceptable excipient.

14. A combination or kit-of-parts comprising components:
(A) a pharmaceutical formulation comprising a β₂-adrenergic receptor agonist, or a pharmaceutically acceptable salt thereof, optionally in admixture with one or more pharmaceutically acceptable excipient, and
(B) a pharmaceutical formulation comprising a therapeutic weight loss agent, optionally in admixture with one or more pharmaceutically acceptable excipient,
wherein components (A) and (B) are each provided in a form that is suitable for administration in conjunction with the other.

15. The formulation, combination or kit-of-parts of any one of claims 13 and 14, wherein the therapeutic weight loss agent is a GLP-1 receptor agonist.

16. The formulation, combination or kit-of-parts of claim 15, wherein the GLP-1 receptor agonist is liraglutide.

17. The β₂-adrenergic receptor agonist for use, use, method, formulation for use, formulation, combination, or kit-of-parts of any of claims 1 to 16, wherein the β₂-adrenergic receptor agonist is a compound selected from the compounds of Table 1, or a pharmaceutically acceptable salt thereof.

18. The β₂-adrenergic receptor agonist for use, use, method, formulation for use, formulation, combination, or kit-of-parts of any of claims 1 to 17, wherein the β₂-adrenergic receptor agonist is: 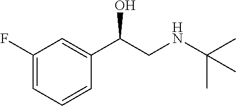
or a pharmaceutically acceptable salt thereof.

19. The β₂-adrenergic receptor agonist for use, use, method, formulation for use, formulation, combination, kit-of-parts of any of claims 1 to 17, wherein the β₂-adrenergic receptor agonist is: 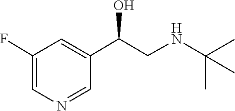
or a pharmaceutically acceptable salt thereof.

20. The β₂-adrenergic receptor agonist for use, method, formulation for use, formulation, combination, kit-of-parts of any of claims 1 to 17, wherein the β₂-adrenergic receptor agonist is: 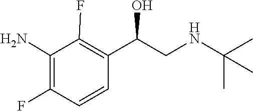 [SMILES: CC(C)(C)NC[C@H](O)c1ccc(F)c(N)c1F]
or a pharmaceutically acceptable salt thereof.
